(19) United States
(12) Patent Application Publication
(10) Pub. No.: US 2020/0377599 A1
(43) Pub. Date: Dec. 3, 2020

(54) COMPOSITIONS AND METHODS FOR TREATING CANCER

(71) Applicant: BETH ISRAEL DEACONESS MEDICAL CENTER, INC., BOSTON, MA (US)

(72) Inventor: WENYI WEI, BOSTON, MA (US)

(73) Assignee: BETH ISRAEL DEACONESS MEDICAL CENTER, INC., BOSTON, MA (US)

(21) Appl. No.: 16/767,487

(22) PCT Filed: Nov. 28, 2018
(86) PCT No.: PCT/US2018/062746

§ 371 Date: May 27, 2020

Related U.S. Application Data
(60) Provisional application No. 62/592,655, filed on Nov. 30, 2017.

(51) Int. Cl. C07K 16/28; C07K 16/40; A61P 35/00

(52) U.S. Cl. CPC C07K 16/2818; A61P 35/00; C07K 16/2827; C07K 16/40

(57) ABSTRACT

Provided are compositions and methods of treating subjects having cancer which involve administering to the subject an anti-PD-L1 or an anti-PD-1 antibody and an inhibitor of cyclin D kinase (CDK)4/6 to treat the cancer. Combining CDK4/6 inhibitor treatment with anti-PD-L1 or anti-PD-1 immunotherapy enhanced tumor regression and dramatically improved overall survival rates in tumor models.

### CROSS-REFERENCE TO RELATED APPLICATION

This application claims the benefit of the following U.S. Provisional Application No. 62/592,655, filed Nov. 30, 2017, the entire contents of which are incorporated herein by reference.

### STATEMENT OF RIGHTS TO INVENTIONS MADE UNDER FEDERALLY SPONSORED RESEARCH

This invention was made with government support under Grant No. GM094777, CA177910, and P50CA101942 awarded by the National Institutes of Health. The government has certain rights in the invention.

### BACKGROUND OF THE INVENTION

Targeting immune checkpoints, such as programmed cell death protein 1 (PD-1) and its ligand PD-L1, have been approved for treating multiple types of human cancer. However, many cancer patients fail to respond to anti-PD-1/PD-L1 treatment and the underlying mechanism(s) for this resistance is not well understood. Recent studies revealed that response to PD-L1 blockade might correlate with PD-L1 expression levels on tumor cells.

Thus, it is important to mechanistically understand the pathways controlling PD-L1 protein expression and stability, which can offer a molecular basis to improve the clinical response rate and efficacy of PD-1/PD-L1 blockade in cancer patients. Accordingly, improved methods for determining the underlying mechanism(s) of PD-L1 expression levels on tumor cells are needed.

### SUMMARY OF THE INVENTION

As described below, the present disclosure features compositions and methods of treating a subject, particularly a mammalian subject, and more particularly, a human subject, who has a cancer (e.g., colon cancer, breast cancer, melanoma, lung cancer, head and neck cancer, prostate cancer). The described compositions and methods embrace the use of an anti-PD-L1 or an anti-PD-1 antibody and an inhibitor of cyclin D kinase 4/6 (CDK4/6) to treat the cancer.

In one aspect, the present invention provides a therapeutic combination comprising a cyclin D kinase 4/6 (CDK4/6) inhibitor and an anti-PD-L1 antibody and/or an anti-PD-1 antibody.

In another aspect, the present invention provides a method of reducing tumor growth, the method involving contacting a tumor cell with a cyclin D kinase 4/6 (CDK4/6) inhibitor and an anti-PD-L1 and/or an anti-PD-1 antibody, thereby reducing tumor growth.

In another aspect, the present invention provides a method of treating cancer in a subject, the method comprising administering to the subject a cyclin D kinase 4/6 (CDK4/6) inhibitor and an anti-PD-L1 and/or an anti-PD-1 antibody, thereby treating cancer in the subject.

In another aspect, the invention features a kit comprising a cyclin D kinase 4/6 (CDK4/6) inhibitor, an anti-PD-L1 and/or an anti-PD-1 antibody.

In various embodiments of any of the above aspects or any other aspect of the invention delineated herein, the CDK4/6 inhibitor is palbociclib, ribociclib, abemaciclib or trilaciclib. In some embodiments of the above aspects, the anti-PD-1 antibody is nivolumab, pembrolizumab, or pidilizumab. In some embodiments of the above aspects, the anti-PD-L1 antibody is MPDL3280A, MEDI4736, BMS-936559, or MSB0010718C. In some embodiments of the above aspects, the combination comprises a CDK4/6 inhibitor and an anti-PD-L1 or an anti-PD-1 antibody. In some embodiments of the above aspects, the combination is formulated in a single composition or is formulated and administered separately. In some embodiments, the combination comprises a CDK4/6 inhibitor (e.g., palbociclib) and an anti-PD1 antibody. In various embodiments, the cancer is colon cancer, breast cancer, melanoma, prostate cancer, lung cancer, and head and neck cancer. In some embodiments of the above aspects, the treatment reduces tumor growth relative to a reference. In some embodiments of the above aspects, the treatment increases survival of the subject.

### Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

By “cyclin D kinase 4 (CDK4)” is meant a protein or fragment thereof having serine/threonine kinase activity, and having at least about 85% identity to NCBI Ref. Seq. NP_000066, which functions in cell cycle regulation.

By “CDK4/6 Inhibitor” is meant an agent that inhibits CDK4 and/or 6 expression, function or activity. Exemplary CDK4/6 inhibitors include, but are not limited to, palbociclib, ribociclib, abemaciclib and trilaciclib.

By “anti-PD-L1 antibody” is meant an antibody, or fragment thereof, that selectively binds a PD-L1 polypeptide. Exemplary anti-PD-L1 antibody is MPDL3280A, MEDI4736, BMS-936559, or MSB0010718C.

By “PD-L1 polypeptide” is meant a polypeptide or fragment thereof having at least about 85%, or greater, amino acid identity to NCBI Accession No. NP_001254635 (SEQ ID NO: 1, below) and having PD-1 binding activity.

### PD-L1 Polypeptide Sequence

### NCBI ACCESSION NO. NP_001254635

| | |
|---|---|
| 1 | MRIFAVFIFM TYWHLLNAPY NKINQRILVV DPVTSEHELT CQAEGYPKAE VIWTSSDHQV |
| 61 | LSGKTTTTNS KREEKLFNVT STLRINTTTN EIFYCTFRRL DPEENHTAEL VIPELPLAHP |
| 121 | PNERTHLVIL GAILLCLGVA LTFIFRLRKG RMMDVKKCGI QDTNSKKQSD THLEET |

By “PD-L1 nucleic acid molecule” is meant a polynucleotide encoding a PD-L1 polypeptide. An exemplary PD-L1 nucleic acid molecule sequence is provided at NCBI Accession No. NM_001267706 and in SEQ ID NO: 2, below.

### PD-L1 Nucleic Acid Sequence

NCBI ACCESSION NO. NM_001267706 mRNA

| | |
|---|---|
| 1 | ggcgcaacgc tgagcagctg gcgcgtcccg cgcggcccca gttctgcgca gcttcccgag |
| 61 | gctccgcacc agccgcgctt ctgtccgcct gcagggcatt ccagaaagat gaggatattt |
| 121 | gctgtcttta tattcatgac ctactggcat ttgctgaacg ccccatacaa caaaatcaac |
| 181 | caaagaattt tggttgtgga tccagtcacc tctgaacatg aactgacatg tcaggctgag |
| 241 | ggctacccca aggccgaagt catctggaca agcagtgacc atcaagtcct gagtggtaag |
| 301 | accaccacca ccaattccaa gagagaggag aagcttttca atgtgaccag cacactgaga |
| 361 | atcaacacaa caactaatga gattttctac tgcactttta ggagattaga tcctgaggaa |
| 421 | aaccatacag ctgaattggt catcccagaa ctacctctgg cacatcctcc aaatgaaagg |
| 481 | actcacttgg taattctggg agccatctta ttatgccttg gtgtagcact gacattcatc |
| 541 | ttccgtttaa gaaaagggag aatgatggat gtgaaaaaat gtggcatcca agatacaaac |
| 601 | tcaaagaagc aaagtgatac acatttggag gagacgtaat ccagcattgg aacttctgat |
| 661 | cttcaagcag ggattctcaa cctgtggttt aggggttcat cggggctgag cgtgacaaga |
| 721 | ggaaggaatg ggcccgtggg atgcaggcaa tgtgggactt aaaaggccca agcactgaaa |
| 781 | atggaacctg gcgaaagcag aggaggagaa tgaagaaaga tggagtcaaa cagggagcct |
| 841 | ggagggagac cttgatactt tcaaatgcct gaggggctca tcgacgcctg tgacagggag |
| 901 | aaaggatact tctgaacaag gagcctccaa gcaaatcatc cattgctcat cctaggaaga |
| 961 | cgggttgaga atccctaatt tgagggtcag ttcctgcaga agtgcccttt gcctccactc |
| 1021 | aatgcctcaa tttgttttct gcatgactga gagtctcagt gttggaacgg gacagtattt |
| 1081 | atgtatgagt ttttcctatt tattttgagt ctgtgaggtc ttcttgtcat gtgagtgtgg |
| 1141 | ttgtgaatga tttcttttga agatatattg tagtagatgt tacaattttg tcgccaaact |
| 1201 | aaacttgctg cttaatgatt tgctcacatc tagtaaaaca tggagtattt gtaaggtgct |
| 1261 | tggtctcctc tataactaca agtatacatt ggaagcataa agatcaaacc gttggttgca |
| 1321 | taggatgtca cctttattta acccattaat actctggttg acctaatctt attctcagac |
| 1381 | ctcaagtgtc tgtgcagtat ctgttccatt taaatatcag ctttacaatt atgtggtagc |
| 1441 | ctacacacat aatctcattt catcgctgta accaccctgt tgtgataacc actattattt |
| 1501 | tacccatcgt acagctgagg aagcaaacag attaagtaac ttgcccaaac cagtaaatag |
| 1561 | cagacctcag actgccaccc actgtccttt tataatacaa tttacagcta tattttactt |
| 1621 | taagcaattc ttttattcaa aaaccattta ttaagtgccc ttgcaatatc aatcgctgtg |
| 1681 | ccaggcattg aatctacaga tgtgagcaag acaaagtacc tgtcctcaag gagctcatag |
| 1741 | tataatgagg agattaacaa gaaaatgtat tattacaatt tagtccagtg tcatagcata |
| 1801 | aggatgatgc gaggggaaaa cccgagcagt gttgccaaga ggaggaaata ggccaatgtg |
| 1861 | gtctgggacg gttggatata cttaaacatc ttaataatca gagtaatttt catttacaaa |
| 1921 | gagaggtcgg tacttaaaat aaccctgaaa aataacactg gaattccttt tctagcatta |
| 1981 | tatttattcc tgatttgcct ttgccatata atctaatgct tgtttatata gtgtctggta |
| 2041 | ttgtttaaca gttctgtctt ttctatttaa atgccactaa attttaaatt catacctttc |
| 2101 | catgattcaa aattcaaaag atcccatggg agatggttgg aaaatctcca cttcatcctc |
| 2161 | caagccattc aagtttcctt tccagaagca actgctactg cctttcattc atatgttctt |
| 2221 | ctaaagatag tctacatttg gaaatgtatg ttaaaagcac gtatttttaa aatttttttc |
| 2281 | ctaaatagta acacattgta tgtctgctgt gtactttgct atttttattt attttagtgt |
| 2341 | ttcttatata gcagatggaa tgaatttgaa gttcccaggg ctgaggatcc atgccttctt |
| 2401 | tgtttctaag ttatctttcc catagctttt cattatcttt catatgatcc agtatatgtt |
| 2461 | aaatatgtcc tacatataca tttagacaac caccatttgt taagtatttg ctctaggaca |
| 2521 | gagtttggat ttgtttatgt ttgctcaaaa ggagacccat gggctctcca gggtgcactg |
| 2581 | agtcaatcta gtcctaaaaa gcaatcttat tattaactct gtatgacaga atcatgtctg |
| 2641 | gaacttttgt tttctgcttt ctgtcaagta taaacttcac tttgatgctg tacttgcaaa |
| 2701 | atcacatttt ctttctggaa attccggcag tgtaccttga ctgctagcta ccctgtgcca |
| 2761 | gaaaagcctc attcgttgtg cttgaaccct tgaatgccac cagctgtcat cactacacag |
| 2821 | ccctcctaag aggcttcctg gaggtttcga gattcagatg ccctgggaga tcccagagtt |
| 2881 | tcctttccct cttggccata ttctggtgtc aatgacaagg agtaccttgg ctttgccaca |
| 2941 | tgtcaaggct gaagaaacag tgtctccaac agagctcctt gtgttatctg tttgtacatg |
| 3001 | tgcatttgta cagtaattgg tgtgacagtg ttctttgtgt gaattacagg caagaattgt |
| 3061 | ggctgagcaa ggcacatagt ctactcagtc tattcctaag tcctaactcc tccttgtggt |
| 3121 | gttggatttg taaggcactt tatccctttt gtctcatgtt tcatcgtaaa tggcataggc |
| 3181 | agagatgata cctaattctg catttgattg tcactttttg tacctgcatt aatttaataa |
| 3241 | aatattctta tttattttgt tacttggtac accagcatgt ccattttctt gtttattttg |
| 3301 | tgtttaataa aatgttcagt ttaacatccc agtggagaaa gttaaaaaa |

By “anti-PD-1 antibody” is meant an antibody, or fragment thereof, that selectively binds a PD-1 polypeptide. In one embodiment, the anti-PD-1 antibody is nivolumab, pembrolizumab, or pidilizumab.

By “PD-1 polypeptide” is meant a polypeptide or fragment thereof having at least about 85%, or greater, amino acid identity to NCBI Accession No. NP_005009.2 (SEQ ID NO: 3, below) and having PD-L1 binding activity.

| | |
|---|---|
| 1 | mqipqapwpv vwavlqlgwr pgwfldspdr pwnpptfspa llvvtegdna tftcsfsnts |
| 61 | esfvinwyrm spsnqtdkla afpedrsqpg qdcrfrvtql pngrdfhmsv vrarrndsgt |
| 121 | ylcgaislap kaqikeslra elrvterrae vptahpspsp rpagqfqtlv vgvvggllgs |
| 181 | lvllvwvlav icsraargti garrtgqplk edpsavpvfs vdygeldfqw rektpeppvp |
| 241 | cvpeqteyat ivfpsgmgts sparrgsadg prsaqplrpe dghcswpl |

By “PD-1 nucleic acid molecule” is meant a polynucleotide encoding a PD-1 polypeptide. An exemplary PD-1 nucleic acid molecule sequence is provided at NCBI Accession No. NG_012110.1 and in SEQ ID NO: 4, below)

### PD-1 Nucleic Acid Sequence

### NCBI ACCESSION NO. NG_012110.1

AGTTTCCCTTCCGCTCACCTCCGCCTGAGCAGTGGAGAAGGCGGCACTCT
GGTGGGGCTGCTCCAGGCATGCAGATCCCACAGGCGCCCTGGCCAGTCGT
CTGGGCGGTGCTACAACTGGGCTGGCGGCCAGGATGGTTCTTAGGTAGGT
GGGGTCGGCGGTCAGGTGTCCCAGAGCCAGGGGTCTGGAGGGACCTTCCA
CCCTCAGTCCCTGGCAGGTCGGGGGGTGCTGAGGCGGGCCTGGCCCTGGC
AGCCCAGGGGTCCCGGAGCGAGGGGTCTGGAGGGACCTTTCACTCTCAGT
CCCTGGCAGGTCGGGGGGTGCTGTGGCAGGCCCAGCCTTGGCCCCCAGCT
CTGCCCCTTACCCTGAGCTGTGTGGCTTTGGGCAGCTCGAACTCCTGGGT
TCCTCTCTGGGCCCCAACTCCTCCCCTGGCCCAAGTCCCCTCTTTGCTCC
TGGGCAGGCAGGACCTCTGTCCCCTCTCAGCCGGTCCTTGGGGCTGCGTG
TTTCTGTAGAATGACGGGTCAGGCTGGCCAGAACCCCAAACCTTGGCCGT
GGGGAGTCTGCGTGGCGGCTCTGCCTTGCCCAGGCATCCTTGGTCCTCAC
TCGAGTTTTCCTAAGGATGGGATGAGCCCCATGTGGGACTAACCTTGGCT
TTACGACGTCAAAGTTTAGATGAGCTGGTGATATTTTTCTCATTATATCC
AAAGTGTACCTGTTCGAGTGAGGACAGTTCTTCTGTCTCCAGGATCCCTC
CTGGGTGGGGATTGTGCCCGCCTGGGTCTCTGCCCAGATTCCAGGGCTCT
CCCCGAGCCCTGTTCAGACCATCCGTGGGGGAGGCCTTGGCCTCACTCTC
CCGGATCGAGGAGAGAGGGAGCCTCTTCCTGGGCTGCCCGTGACCCTGGG
CCCTCTGTGTACACTGTGACCACAGCCCGCTCCTGGACCCTCTGTGCCCG
GCTGGCCCTCTGTGCCCAGCCAGCCTGCACCTGGGGATGCCAAGGCCTGG
GGAGGGTGGTTTCACCCAGGCCAAGCCTAAGACAGTCCCTCTGGGCCCTG
CTGGGTACCGGGGTGTGACACCACTGGGAGGACAAGATGAGGGGCACCCC
TGGGGCCGCCCTGACACCCCCTCGAGGCTCCTGCCCCGGGGGTCCTGGTG
CCCCTTCACTGTGGCAGGCGACTGGGGGTTCCCCACCTCGGCCCCTCTCC
CGGGGCCTGCTCCCCGGCACCTGAGGCAGCATCCTTGTCAGGGCCGTGCC
TTCCTGCCTCAGCGCCACCTCTTAAGGTTGGCCCGTGGGTCACTCAGGAC
TCAGAACTGGAGATTCTGGGCAAAAGGCAAAGAGCAAAGGGCCAAAAGGC
ATCCCAGGGAGACGACTGCGGGGGAACCAGAGGGCAGAGGGGCGCTCGTC
ACAGGGGAGGGGGAGCTGAGCGAGGCAGGAGGGGAGCCGAGCCTCTCCCC
CCGTGTCCCGGCTCTTCAGGCACGCCCTCGGGACGCCACCCTCCCCGACC
CAGGCGGGAAAGATAAGAGCAAGGTGTCCGCAGCCTGACACTCGTGCCTC
AGGTGCCCGCGCTTGTGCCGGACAAGACTCTCACAGGTGGCATGCCTCGG
TTTCCCCACTGGTAACAGCACAGGGCACTCAGCAAGGCGCAGTGGGCATG
ACTGGGGTCCTGTGGGTCCTGACCCAGATGTGGCCACCCCGGCCGCAGTG
GTCTTCATTCCAGGATGCCTCTTTTCCCTCCTGATCTATTCACTGCGTTC
GCCATTCGGTCATTCCCGGGGCCACCACTCCCACCTCAGGTGTGTGCTTC
CCTTGTGTTTTATGAGATATCCCCAACCCGGCTGCTTATTGGCCCCGTCC
GAGGGCAGGAGCATAAATAAGAGCCTCTGCTTTGGCGTGGGACCACTGTG
AGCTCCAGTCAGCGCTGCCACTGCTGAGCTCTGGGCCTTCGACAGGACTT
GGCCCCTTACTGACTTCTCCGTGTGCTTTGGGTCATGGGTGAGGACGCCT
CCTGGCAAGGCTGCGTCCTGAGGATTAAATCGGGTCATCTGTGAAAACTA
CCCAGCCCAGCCCCTGACACTTTTTTGTTTGTTTCTTTTAGTGACAGGGT
CTTGCTCTGTCACCCAGGCTGGAGTGCAGTGGTGTGATCTCGGCTCACTC
GACCTCCGGGGCTCAAGCAATTCTCCCACCTCTGCCTCCAGAGTAGCTGG
GACTATAGGCACGTGCCACCCTGCCAGGCTAATTTCTTCCATTTTTTTTA
GAGACAGGGTCTCGCTATGTTCCCCAGGCTGGGCTCAAATGGTCCTCCCA
CCTCAGCCTCCCCAAGTACTGGGATTACAGGCATAAGCCACTGCATCTGG
CCTCCATGACACATATTTTTAAAGTCTGATTTTTAAAGTCAAACTTTTGA
AGTCAGATTTTAAACGGACTATTTTGAAAAATATACAAAAACGTTTAAAA
ACAATGAATATCCCTCACCTAGAATCAATAACTAAGAATATTGACACATT
TGCTTTGGGGACTGGGCGGCTGGAGCTGCCATGACAAAGCTCCGCCGACC
GAGTGGCTTTTAAACAGAGCCTGCCCTCTCGCCGACTGAGGGCTGGACGT
GCAGGATGGAGCTCCGCAGGGTCGGCTCCCCTGTGCTCTGAGGGGCTCTG
CTCAGCCTCTCCCGGCTGTGGCTTAAAAACAGAGCCTGTCCTCCCGCCGT
GGGGGGCTGGACATGCAGGACCGAGGGGCCACAGGGTCGGCTCCCTGTGC
TCCGAGAGGGCTCTGCTCAGCTTCTCCTGGCTGGGGGGTTTTGTGGCCA
CCCTCTGTGTTCCTGGGTTCAGAAGCATCCCCCAGGCTCTGCCTTCATC
TCTGCACGGGTGACTCTGTACAGGAAGCCAGGCCTGCTGGTCAATGGCC
ACCCAGCCCTGTGCCCTCATCTTACCTAGTCCCAGCTGCCGTCACCCTA
TTCCTAATAAGGCCGCCTTCTGAGGTCATGGGGTTAGGACTTCCACATA
GGAATCTGTGGGGACACGGTTCGGCCCACAGCCCTTCCCACCTCCACAC
ACACACACGACTGTGAGGAGTTGGAAGACCTCACTCCTCACCCCTGCCA
GGTCCTCTAGGGACAAGCTCGCTGTCCTCATCCCAGCACAGCCCGTGGG
ACGGTTTCCTTGTCCCTAATGGGACCACGGTCAGAGATGCCGGGTCTGG
TCTGGGCCAGCAGGTTCCTCCGCCCGGGGCAGGCAGCCTTCTTCTGTGC
GCTTCTGGAAAGCAATGTCCTGTAATGCGGTCTCTCTGCGGGAGCACCC
CCACCGCCACCTCACAGGCCTGTTCCACAGCCCCGGGATGGGCTCTGTC
TCCCTCCTGACCCTGCATAGGGCACAGCCCTCTCTCATCAACCCACGAT
CCTACGTGGATCCGAGAGGGAGCACCTGGGGAAACAATGGAATCCCATA
GAAACACCCCAAATCTAACTTGATCCAGGACCAGCCAGTGGTCACTTCT
GAATATTCACCTTCCTAGTAGACACTACCAGCCAAGGGAGGCCAGGAAG
CCTTCCTGGAGGAGGTGGCCTGAGGACTGGGGTGAGGCAGGCCCTGCGT
GGGGGTCGCCACCCAGCACCCCCACACTGGGTGGGAGCCAGTCTCTGAG
ACTGGCTGGGGGAGGTGGGAGAGGGGGCTGCTTGAACTGCAGACACCGA
GGTCTAGCCCCCACCCCACCCAGCCAGTTGGTGGAGGCAGGGGAGGCCG
AGGGGCCCAGCTGGACCTGCTCCCCGGGGTGGATTCCAAAATAGGGGGG
TTGGGGGGGGCGGAACAGGAGCCCAGGGTCCTGGCTTGAGGCCCAGTGG
CTGAGGGCTGGTGCAAGCCAGACAGGAAAAGGGTTGAGCCTGTCAGCGC
CAGCACAGATCAAGTCAGGAGCAGGTCCCTCCACCAATGTGTGCAAATA
AATAGCAGCTAAGTTTCCAGTTACAAGAACAATGCACAGATGGTCCCAG
GGACATTGCGGTGTGGACACACAGCGGCCATTGTCCTGTCGCCAGCACC
TCGCCCTACAGCTGGGGGGTCCCTTAGCACTTCCTAGCCATGCAGGGTC
CCTGCTCACAGTACCCGTGATGACTTCTGTTCCTCACCTGCCTGTCTGT
CCCGACAGCTGCATGGCAGCCCTGGCCTGGGAGATGGAGACCCCGAGGG
GCTGCCTGCGGTGGTGGGGCCCCTGGGTCCCCACTGCATTCCCAGAAAC
CCAGAGGGCAGGGCATTTCCCCTGCTCTGTGCCGAGTCCACCCAGCCCC
AGCCTAGGCCCAGTAAGGGCTGCAGCCCACCCTGTCCCAGGCTGCCTCC
CAGGAGCCCTCTTGGCCCTGATGCCAGAAGCCCATCTTCCTCCATTCAG
GCAGGTCTCTGAGTGCCCTGGCCTGGCTGCCTGCTGGCCCTGAGAGTCA
CACTACCCCACAGCCCTCCTTGGTCAAAATCCACTCTGGAGTGGCTGGA
AGATTCCCCGGGCCCACGCCGCACACGCCTATGCAGGGAGCTTCCCCTG
GCCGGCCGGCAGACAAGGGCGGTCTCAGAGAGGGGGCTCACCTCAGCAG
CCCCTTGTGTAGCTGGCCCTCGCCCCTGCCACCTCTGGGAACACCACCA
GGAAGCTGGGGGACAGGCACGCAGGTGAAGGAGGCGAGCGCTTGTCAGC
CGGGAGGCCATGGGCACAGAGGGAACAGGGACACCCTGGGTGGCCTCAA
GGTCACTTCAAACCCCTCACTCGTCCCCTGGGAGGGTGCCCAGTGAGGT
TGGCACTAGGAGTTGGTCCTGGTCACATGACAGACCCACCCACCTCTGG
TGTCCAGCCAGCACGCCGTGGGCCAGCCTGGCTGCAGGGACACGAGGGC
AGCAGCCCCCTCCTCCTCTGAGCTGGTTGCTCCTTGAGTCATCACCACC
GCCTGCCACGGAGGCCGCCTGTCCCAGGAAGCAGAGGGACCGCAGCTGT
GGCAACCAGGGCCTGGTCTCTGTGTCACCTCGCTGGGGGGCCGTGCCCA
GGCCTGAGACGGAACTGAGTGACAGTGCACTGGGTCTGACAGTGTGGGG
CTGGCGCCATGTTTGGGGAACCCTGTGGCATGGGACCTGTGGGTGAGCC
GGGAAAATCACCCCGTTGCATGGCATCTCGGGCCTGGATCTTAAGCGCC
TGTGTTGGTGCCTCCGCCTGGCGGAAGAGCCGCGACCCCCACGTTGCCA
TGCGGGTATCCCAAGCCCTGACCCTGGCAGGCATATGTTTCAGGAGGTC
CTTGTCTTGGGAGCCCAGGGTCGGGGGCCCCGTGTCTGTCCACATCCGA
GTCAATGGCCCATCTCGTCTCTGAAGCATCTTTGCTGTGAGCTCTAGTC
CCCACTGTCTTGCTGGAAAATGTGGAGGCCCCACTGCCCACTGCCCAGG
GCAGCAATGCCCATACCACGTGGTCCCAGCTCCGAGCTTGTCCTGAAAA
GGGGGCAAAGACTGGACCCTGAGCCTGCCAAGGGGCCACACTCCTCCCA
GGGCTGGGGTCTCCATGGGCAGCCCCCCACCCACCCAGACCAGTTACAC
TCCCCTGTGCCAGAGCAGTGCAGACAGGACCAGGCCAGGATGCCCAAGG
GTCAGGGGCTGGGGATGGGTAGCCCCCAAACAGCCCTTTCTGGGGGAAC
TGGCCTCAACGGGGAAGGGGGTGAAGGCTCTTAGTAGGAAATCAGGGAG
ACCCAAGTCAGAGCCAGGTGCTGTGCAGAAGCTGCAGCCTCACGTAGAA
GGAAGAGGCTCTGCAGTGGAGGCCAGTGCCCATCCCCGGGTGGCAGAGG
CCCCAGCAGAGACTTCTCAATGACATTCCAGCTGGGGTGGCCCTTCCAG
AGCCCTTGCTGCCCGAGGGATGTGAGCAGGTGGCCGGGGAGGCTTTGTG
GGGCCACCCAGCCCCTTCCTCACCTCTCTCCATCTCTCAGACTCCCCAG
ACAGGCCCTGGAACCCCCCCACCTTCTCCCCAGCCCTGCTCGTGGTGAC
CGAAGGGGACAACGCCACCTTCACCTGCAGCTTCTCCAACACATCGGAG
AGCTTCGTGCTAAACTGGTACCGCATGAGCCCCAGCAACCAGACGGACA
AGCTGGCCGCCTTCCCCGAGGACCGCAGCCAGCCCGGCCAGGACTGCCG
CTTCCGTGTCACACAACTGCCCAACGGGCGTGACTTCCACATGAGCGTG
GTCAGGGCCCGGCGCAATGACAGCGGCACCTACCTCTGTGGGGCCATCT
CCCTGGCCCCCAAGGCGCAGATCAAAGAGAGCCTGCGGGCAGAGCTCAG
GGTGACAGGTGCGGCCTCGGAGGCCCCGGGGCAGGGGTGAGCTGAGCCG
GTCCTGGGGTGGGTGTCCCCTCCTGCACAGGATCAGGAGCTCCAGGGTC
GTAGGGCAGGGACCCCCCAGCTCCAGTCCAGGGCTCTGTCCTGCACCTG
GGGAATGGTGACCGGCATCTCTGTCCTCTAGCTCTGGAAGCACCCCAGC
CCCTCTAGTCTGCCCTCACCCCTGACCCTGACCCTCCACCCTGACCCCG
TCCTAACCCCTGACCTTTGTGCCCTTCCAGAGAGAAGGGCAGAAGTGCC
CACAGCCCACCCCAGCCCCTCACCCAGGCCAGCCGGCCAGTTCCAAACC
CTGGTGGTTGGTGTCGTGGGCGGCCTGCTGGGCAGCCTGGTGCTGCTAG
TCTGGGTCCTGGCCGTCATCTGCTCCCGGGCCGCACGAGGTAACGTCAT
CCCAGCCCCTCGGCCTGCCCTGCCCTAACCCTGCTGGCGGCCCTCACTC
CCGCCTCCCCTTCCTCCACCCTTCCCTCACCCCACCCCACCTCCCCCCA
TCTCCCCGCCAGGCTAAGTCCCTGATGAAGGCCCCTGGACTAAGACCCC
CCACCTAGGAGCACGGCTCAGGGTCGGCCTGGTGACCCCAAGTGTGTTT
CTCTGCAGGGACAATAGGAGCCAGGCGCACCGGCCAGCCCCTGGTGAGT
CTCACTCTTTTCCTGCATGATCCACTGTGCCTTCCTTCCTGGGTGGGCA
GAGGTGGAAGGACAGGCTGGGACCACACGGCCTGCAGGACTCACATTCT
ATTATAGCCAGGACCCCACCTCCCCAGCCCCCAGGCAGCAACCTCAATC
CCTAAAGCCATGATCTGGGGCCCCAGCCCACCTGCGGTCTCCGGGGGTG
CCCGGCCCATGTGTGTGCCTGCCTGCGGTCTCCAGGGGTGCCTGGCCCA
CGCGTGTGCCCGCCTGCGGTCTCTGGGGGTGCCCGGCCCACATATGTGC
CTGCCTGCGGTCTCCAGGTGTGCCCGGCCCATGCGTGTGCCCACCTGCG
AGGGCGTGGGGTGGGCTTGGTCATTTCTTATCTTACATTGGAGACAGGA
GAGCTTGAAAAGTCACATTTTGGAATCCTAAATCTGCAAGAATGCCAGG
GACATTTCAGAGGGGGACATTGAGCCAGAGAGGAGGGGTGGTGTCCCCA
GATCACACAGAGGGCAGTGGTGGGACAGCTCAGGGTAAGCAGCTCATAG
TGGGGGGCCCAGGTTCGGTGCCGGTACTGCAGCCAGGCTGTGGAGCCGC
GGGCCTCCTTCCTGCGGTGGGCCGTGGGGCTGACTCCCTCTCCCTTTCT
CCTCAAAGAAGGAGGACCCCTCAGCCGTGCCTGTGTTCTCTGTGGACTA
TGGGGAGCTGGATTTCCAGTGGCGAGAGAAGACCCCGGAGCCCCCCGTG
CCCTGTGTCCCTGAGCAGACGGAGTATGCCACCATTGTCTTTCCTAGCG
GAATGGGCACCTCATCCCCCGCCCGCAGGGGCTCAGCTGACGGCCCTCG
GAGTGCCCAGCCACTGAGGCCTGAGGATGGACACTGCTCTTGGCCCCTC
TGACCGGCTTCCTTGGCCACCAGTGTTCTGCAGACCCTCCACCATGAGC
CCGGGTCAGCGCATTTCCTCAGGAGAAGCAGGCAGGGTGCAGGCCATTG
CAGGCCGTCCAGGGGCTGAGCTGCCTGGGGGCGACCGGGGCTCCAGCCT
GCACCTGCACCAGGCACAGCCCCACCACAGGACTCATGTCTCAATGCCC
ACAGTGAGCCCAGGCAGCAGGTGTCACCGTCCCCTACAGGGAGGGCCAG
ATGCAGTCACTGCTTCAGGTCCTGCCAGCACAGAGCTGCCTGCGTCCAG
CTCCCTGAATCTCTGCTGCTGCTGCTGCTGCTGCTGCTGCTGCCTGCGG
CCCGGGGCTGAAGGCGCCGTGGCCCTGCCTGACGCCCCGGAGCCTCCTG
CCTGAACTTGGGGGCTGGTTGGAGATGGCCTTGGAGCAGCCAAGGTGCC
CCTGGCAGTGGCATCCCGAAACGCCCTGGACGCAGGGCCCAAGACTGGG
CACAGGAGTGGGAGGTACATGGGGCTGGGGACTCCCCAGGAGTTATCTG
CTCCCTGCAGGCCTAGAGAAGTTTCAGGGAAGGTCAGAAGAGCTCCTGG
CTGTGGTGGGCAGGGCAGGAAACCCCTCCACCTTTACACATGCCCAGGC
AGCACCTCAGGCCCTTTGTGGGGCAGGGAAGCTGAGGCAGTAAGCGGGC
AGGCAGAGCTGGAGGCCTTTCAGGCCCAGCCAGCACTCTGGCCTCCTGC
CGCCGCATTCCACCCCAGCCCCTCACACCACTCGGGAGAGGGACATCCT
ACGGTCCCAAGGTCAGGAGGGCAGGGCTGGGGTTGACTCAGGCCCCTCC
CAGCTGTGGCCACCTGGGTGTTGGGAGGGCAGAAGTGCAGGCACCTAGG
GCCCCCCATGTGCCCACCCTGGGAGCTCTCCTTGGAACCCATTCCTGAA
ATTATTTAAAGGGGTTGGCCGGGCTCCCACCAGGGCCTGGGTGGGAAGG
TACAGGCGTTCCCCCGGGGCCTAGTACCCCCGCCGTGGCCTATCCACTC
CTCACATCCACACACTGCACCCCCACTCCTGGGGCAGGGCCACCAGCAT
CCAGGCGGCCAGCAGGCACCTGAGTGGCTGGGACAAGGGATCCCCCTTC
CCTGTGGTTCTATTATATTATAATTATAATTAAATATGAGAGCATGCTA
AGGA

By “agent” is meant any small molecule chemical compound, antibody, nucleic acid molecule, or polypeptide, or fragments thereof.

By “ameliorate” is meant decrease, suppress, attenuate, diminish, arrest, or stabilize the development or progression of a disease, such as cancer.

By “alteration” is meant a change (increase or decrease) in the expression levels or activity of a gene or polypeptide as detected by standard art known methods such as those described herein. As used herein, an alteration includes a 10% change in expression or activity levels, preferably a 25% change, more preferably a 40% change, and most preferably a 50% or greater change in expression levels.”

By “analog” is meant a molecule that is not identical, but has analogous functional or structural features. For example, a polypeptide analog retains the biological activity of a corresponding naturally-occurring polypeptide, while having certain biochemical modifications that enhance the analog's function relative to a naturally occurring polypeptide. Such biochemical modifications could increase the analog's protease resistance, membrane permeability, or half-life, without altering, for example, ligand binding. An analog may include an unnatural amino acid.

In this disclosure, “comprises,” “comprising,” “containing,” and “having” and the like can have the meaning ascribed to them in U.S. Patent law, and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

“Detect” refers to identifying the presence, absence or amount of the analyte to be detected.

By “disease” is meant any condition or disorder that damages or interferes with the normal function of a cell, tissue, or organ. In a disease, such as cancer (e.g., colon cancer, breast cancer, melanoma, lung cancer, head and neck cancer, prostate cancer), the normal function of a cell tissue or organ is subverted to enable immune evasion and/or escape.

By “effective amount” is meant the amount of a required to ameliorate the symptoms of a disease relative to an untreated patient. The effective amount of active compound(s) used to practice the present invention for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount. In one embodiment, an effective amount of an agent defined herein is sufficient to reduce or stabilize the proliferation of a cancer cell. In another embodiment, an effective amount of an agent defined herein is sufficient to kill a cancer cell.

The invention provides a number of targets that are useful for the development of highly specific drugs to treat a disorder characterized by the methods delineated herein. In addition, the methods of the invention provide a facile means to identify therapies that are safe for use in subjects. In addition, the methods of the invention provide a route for analyzing virtually any number of compounds for effects on a disease described herein with high-volume throughput, high sensitivity, and low complexity.

By “fragment” is meant a portion of a polypeptide or nucleic acid molecule. This portion contains, preferably, at least 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide of this invention is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

By “isolated polynucleotide” is meant a nucleic acid (e.g., a DNA) that is free of the genes which, in the naturally-occurring genome of the organism from which the nucleic acid molecule of the invention is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule that is transcribed from a DNA molecule, as well as a recombinant DNA that is part of a hybrid gene encoding additional polypeptide sequence.

By an “isolated polypeptide” is meant a polypeptide of the invention that has been separated from components that naturally accompany it. Typically, the polypeptide is isolated when it is at least 60%, by weight, free from the proteins and naturally-occurring organic molecules with which it is naturally associated. Preferably, the preparation is at least 75%, more preferably at least 90%, and most preferably at least 99%, by weight, a polypeptide of the invention. An isolated polypeptide of the invention may be obtained, for example, by extraction from a natural source, by expression of a recombinant nucleic acid encoding such a polypeptide; or by chemically synthesizing the protein. Purity can be measured by any appropriate method, for example, column chromatography, polyacrylamide gel electrophoresis, or by HPLC analysis.

By “marker” is meant any protein or polynucleotide having an alteration in expression level or activity that is associated with a disease or disorder.

As used herein, “obtaining” as in “obtaining an agent” includes synthesizing, purchasing, or otherwise acquiring the agent.

By “reduces” is meant a negative alteration of at least 10%, 25%, 50%, 75%, or 100%.

By “reference” is meant a standard or control condition.

By “specifically binds” is meant a compound or antibody that recognizes and binds a polypeptide of the invention, but which does not substantially recognize and bind other molecules in a sample, for example, a biological sample, which naturally includes a polypeptide of the invention.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, more preferably 80% or 85%, and more preferably 90%, 95% or even 99% identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence.

By “subject” is meant a mammal, including, but not limited to, a human or non-human mammal, such as a bovine, equine, canine, ovine, or feline.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

Unless specifically stated or obvious from context, as used herein, the term “or” is understood to be inclusive. Unless specifically stated or obvious from context, as used herein, the terms “a”, “an”, and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

### BRIEF DESCRIPTION OF THE DRAWINGS

FIG. 1A, FIG. 1B, FIG. 1C, FIG. 1D, FIG. 1E, FIG. 1F, FIG. 1G, and FIG. 1H are images showing the protein abundance of PD-L1 as the protein fluctuates during cell cycle progression. FIG. 1A shows an immunoblot (IB) of whole cell lysates (WCL) derived from HeLa cells synchronized in M phase by nocodazole treatment prior to releasing back into the cell cycle for the indicated times. FIG. 1B are the cell-cycle profiles in FIG. 1A, which were monitored by fluorescence-activated cell sorting (FACS) analysis of DNA content. FIG. 1C shows an IB of WCL derived from HeLa cells synchronized in late G1/S phase by double thymidine blocking prior to releasing back into the cell cycle for the indicated times. FIG. 1D shows the cell-cycle profiles in FIG. 1C, which were analyzed by FACS for DNA content. FIG. 1E and FIG. 1F show IB analysis of WCL derived from MC38 tumor cells, which are derived from colon adenocarcinoma, or CT26 mouse tumor cells, which are colon carcinoma cells, treated with the indicated concentration of nocodazole for 20 hours before harvesting. FIG. 1G and FIG. 1H show IB analysis of WCL derived from 4T1 tumor cells, which are breast cancer cells, or CT26 mouse tumor cells treated with indicated concentration of taxol for 20 hours before harvesting.

FIG. 2A, FIG. 2B, FIG. 2C, FIG. 2D, FIG. 2E, FIG. 2F, FIG. 2G, FIG. 2H, FIG. 2I, FIG. 2J, FIG. 2K, and FIG. 2L are images showing that Cyclin D/CDK4 negatively regulates PD-L1 protein stability. FIG. 2A shows an IB analysis of WCL derived from wild type MEFs and cyclin D1^{-/-}D2^{-/-}D3^{-/-} MEFs. FIG. 2B shows an IB analysis of WCL derived from wild type MEFs, cyclin D1^{-/-}MEFs, cyclin D2^{-/-}MEFs and cyclin D3^{-/-}MEFs. FIG. 2C shows an IB analysis of WCL derived from wild type and cdk4^{-/-}MEFs. FIG. 2D and FIG. 2E show IB analysis of WCL derived from mouse mammary tumors induced by MMTV-Wnt1 or MMTV-c-Myc with/without genetic depletion of cyclin D1. FIG. 2F and

FIG. 2G show IB analysis of WCL derived from Rb-proficient breast cancer cell line: MDA-MB-453, MDA-MB-231, Hs578T and Rb-deficient breast cancer cell line: BT549, MDA-MB-468 and MDA-MB-436 treated with CDK4/6 inhibitor, palbociclib (1 μM), for 48 hours (DMSO as a negative control). FIG. 2H shows quantification of PD-L1 protein band intensity in FIG. 7A through the ImageJ software to demonstrate that PD-L1 protein abundance in various indicated tissues of mice is elevated after palbociclib (150 mg/kg body weight, by gastric gavage) for 7 days. Data were represented as mean±S.D., N=5, and *p<0.05 (Student's t-test). FIG. 2I and FIG. 2J are graphs showing the relative cell surface PD-L1 expression and absolute CD3⁺ T-cell populations from isolated tumor-infiltrating lymphocytes in MC38 tumors or B16-F10 tumors, which are a melanoma model. Mice were treated with vehicle or palbociclib for 7 days prior to FACS analysis. Data are represented as mean±S.D., N=5, and *p<0.05 (Student's t-test). FIG. 2K shows an immunofluorescence staining of PD-L1 and CD3 in mouse mammary tumors induced by MMTV-ErbB2 treated with vehicle or CDK4/6 inhibitor palbociclib as described in Method. The scale bar represents 50 μm. FIG. 2L is a graph showing quantification of the CD3⁺ T cell population in FIG. 2K. Data are represented as mean±S.D., N=4, and *p<0.05 (Student's t-test).

FIG. 3A, FIG. 3B, FIG. 3C, FIG. 3D, FIG. 3E, FIG. 3F, FIG. 3G, FIG. 3H, FIG. 3I, FIG. 3J, FIG. 3K, FIG. 3L, FIG. 3M, FIG. 3N, FIG. 3O, and FIG. 3P are images showing Cullin 3^{SPOP} is the physiological E3 ubiquitin ligase for PD-L1. FIG. 3A shows an IB analysis of WCL derived from C42 cells treated with MG132 (10 μM) or MLN4924 (1 μM) for 12 hours before harvesting. FIG. 3B shows an IB analysis of immunoprecipitates (IP) and WCL derived from 293T cells transfected with HA-PD-L1 and Myc-Cullins constructs as indicated and treated with MG132 (10 μM) for 12 hours before harvesting. FIG. 3C shows an IB analysis of WCL derived from PC3 infected with indicated lentiviral shRNAs against Cullin 3 and selected with puromycin (1 μg/ml) for 72 hours before harvesting. FIG. 3D shows an IB analysis of IP and WCL derived from 293T cells transfected with HA-PD-L1 and Flag-tagged Cullin 3 family adaptor protein constructs as indicated and treated with MG132 (10 μM) for 12 hours before harvesting. FIG. 3E shows an IB analysis of IP and WCL derived from PC3 cells treated with MG132 (30 μM) for 6 hours before harvesting. FIG. 3F shows an IB analysis of WCL derived from Spop^{+/+} and Spop^{-/-} MEFs cells with indicated antibodies. FIG. 3G shows an IB analysis of WCL derived from C42 cells depleted SPOP with indicated sgRNAs. FIG. 3H shows an IB analysis of WCL derived from C42 cells stably infected with lenti-HA-WT, F102C and W131G mutant forms of SPOP, as well as empty vector (EV) as control. FIG. 3I shows an IB analysis of IP and WCL derived from 293T cells transfected with HA-PD-L1 and Flag-tagged SPOP-WT, Y87C, F102C and W131G constructs and treated with MG132 (10 μM) for 12 hours before harvesting. FIG. 3J shows an IB of WCL and Ni-NTA pull-down products derived from the lysates of PC3 cells transfected with the indicated constructs. Cells were treated with 30 μM MG132 for 6 hours before harvesting. FIG. 3K is a graph showing the excised tumor mass (mg) of B16-F10 tumors ectopically expressing SPOP Wild Type (WT) or SPOP-F102C mutant. The B16-F10 tumors were harvested after euthanizing the mice and tumor weight was recorded at the time of sacrifice. Data are represented as mean±S.D., N=5, and *p<0.05 (Student's t-test). FIG. 3L and FIG. 3M are graphs showing the relative cell surface PD-L1 expression and absolute CD3⁺ T-cell populations from the isolated tumor-infiltrating lymphocytes in B16-F10 xenografted tumors ectopically expressing SPOP-WT or the SPOP-F102C mutant as determined by FACS analysis. Data are represented as mean±S.D., N=5, and *p<0.05 (Student's t-test). FIG. 3N shows representative images of PD-L1 and CD8 immunohistochemical staining in SPOP wild-type or mutant primary human prostate cancer samples. The scale bar represents 100 μm (upper panel) or 20 μm (lower panel), respectively. FIG. 3O and FIG. 3P are graphs showing the quantification of IHC analysis of PD-L1 protein levels and CD8⁺ T cells in 82 cases of SPOP wild-type versus 15 cases of SPOP-mutant human prostate tumor specimens. **p<0.01 (Mann-Whitney test for PD-L1); *p<0.05 (Student's t-test for CD8).

FIG. 4A, FIG. 4B, FIG. 4C, FIG. 4D, FIG. 4E, FIG. 4F, FIG. 4G, FIG. 4H, FIG. 4I, FIG. 4J, FIG. 4K, FIG. 4L, FIG. 4M, and FIG. 4N are graphs and images showing that cyclin D/CDK4-mediated phosphorylation of SPOP stabilizes SPOP largely through recruiting 14-3-3γ to disrupt its binding with Cdh1. FIG. 4A shows an IB of WCL derived from HeLa cells with/without depletion of SPOP by sgRNA synchronized in M phase by nocodazole treatment prior to releasing for the indicated times. FIG. 4B shows an IB analysis of IP and WCL derived from MDA-MB-231 cells treated with MG132 (30 μM) for 6 hours before harvesting. FIG. 4C shows an IB analysis of IP and WCL derived from 293T cells transfected with indicated constructs and treated with MG132 (10 μM) for 12 hours before harvesting. FIG. 4D shows an IB of WCL and Ni-NTA pull-down products derived from the lysates of HeLa cells transfected with the indicated constructs. Cells were treated with 30 μM MG132 for 6 hours before harvesting. FIG. 4E shows an IB of WCL derived from HeLa cells with/without depletion of Cdh1 by shRNA synchronized in M phase by nocodazole treatment prior to releasing for the indicated time points. FIG. 4F are in vitro kinase assays showing that cyclin D1/CDK4 phosphorylates SPOP at Ser6, not Ser222. FIG. 4G and FIG. 4H show IB analysis of IP and WCL derived from 293T cells transfected with indicated constructs and treated with MG132 (10 μM) for 12 hours before harvesting. FIG. 4I shows an IB analysis of IP and WCL derived from 293T cells transfected with indicated constructs and treated with MG132 (10 μM) or with/without palbociclib (1 μM) as indicated for 12 hours before harvesting. FIG. 4J shows an IB of WCL derived from HeLa cells with/without depletion of SPOP by sgRNA treated with increased concentration of palbociclib (0, 0.5, 1 μM) for 24 hours before harvesting. FIG. 4K shows graphs of MC38 tumor-bearing mice that were enrolled in different treatment groups as indicated. Tumor volumes of mice treated with control antibody (top left, n=15), anti-PD-1 mAb (top right; n=15), the CDK4/6 inhibitor, palbociclib (lower left; n=14) or combined therapy (lower right; n=12). Tumor volume were measured every three days and plotted individually. FIG. 4M shows graphs of CT26 tumor-bearing mice that were enrolled in different treatment groups as indicated. Tumor volumes of mice treated with control antibody (top left, n=13), anti-PD-1 mAb (top right; n=14), the CDK4/6 inhibitor, palbociclib (lower left; n=12) or combined therapy (lower right; n=12). Tumor volume were measured every three days and plotted individually. FIG. 4L and FIG. 4N show Kaplan-Meier survival curves for each treatment group demonstrate the improved efficacy of combining PD-1 mAb with the CDK4/6 inhibitor, palbociclib. *p<0.05 or **p<0.001 (Gehan-Breslow-Wilcoxo test).

FIG. 5A, FIG. 5B, FIG. 5C, FIG. 5D, FIG. 5E, FIG. 5F, FIG. 5G, FIG. 5H, and FIG. 5I are graphs and images showing that PD-L1 fluctuates during cell cycle progression. FIG. 5A and FIG. 5B show graphs of quantitative real-time PCR (qRT-PCR) analyses of relative mRNA levels of PD-L1 and GAPDH from samples derived from HeLa cells synchronized in M phase by nocodazole treatment prior to releasing back to the cell cycle for the indicated time points. FIG. 5C and FIG. 5D show an immunoblot (IB) of whole cell lysates (WCL) derived from MDA-MB-231 or HCC1954 cells synchronized in M phase by nocodazole treatment prior to releasing back into the cell cycle for the indicated times. FIG. 5E shows an

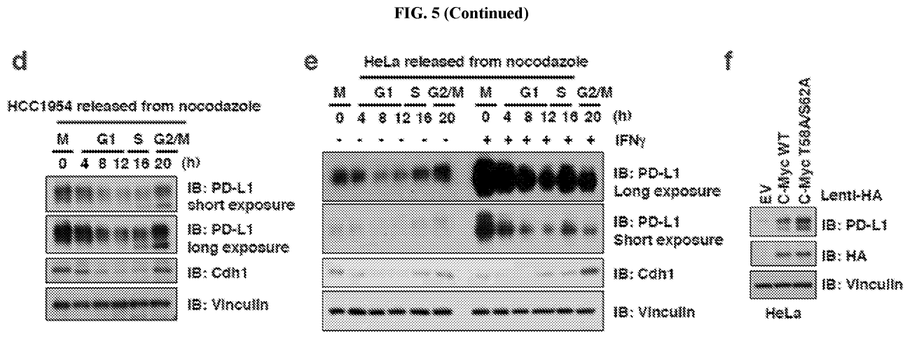

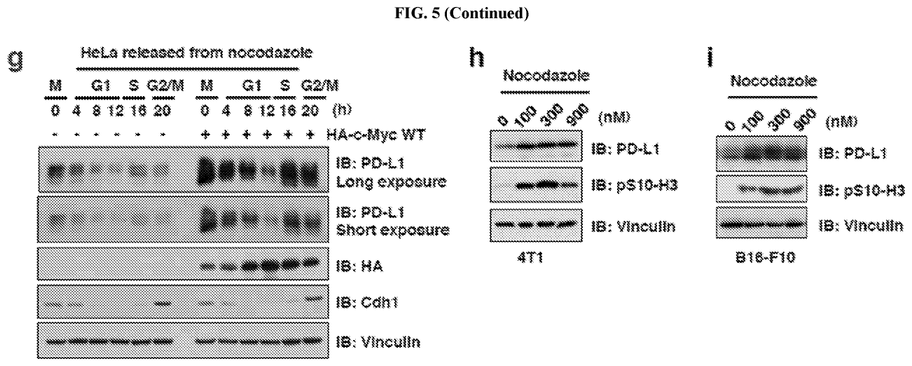

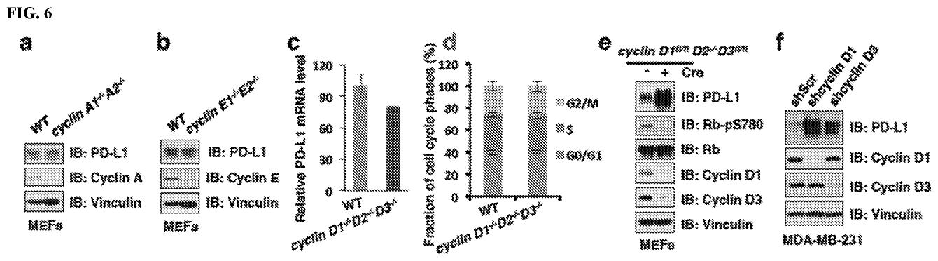

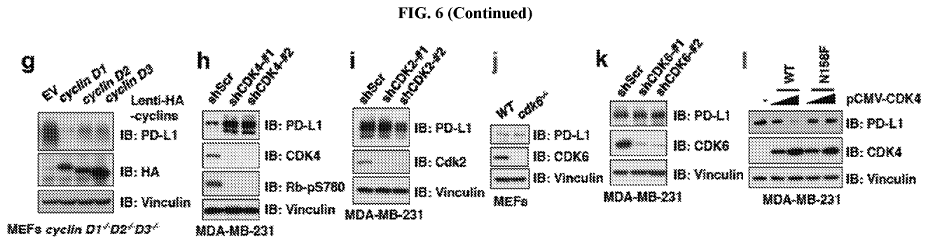

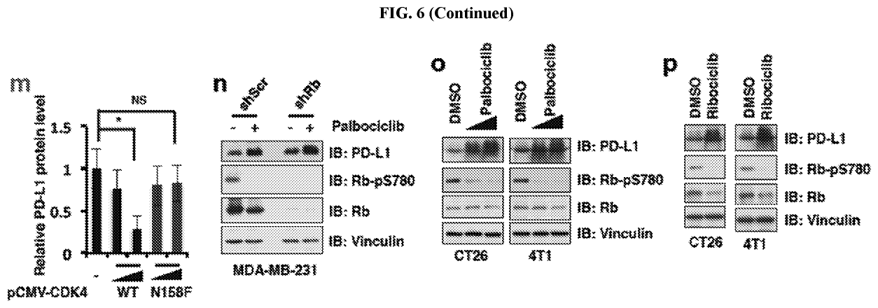

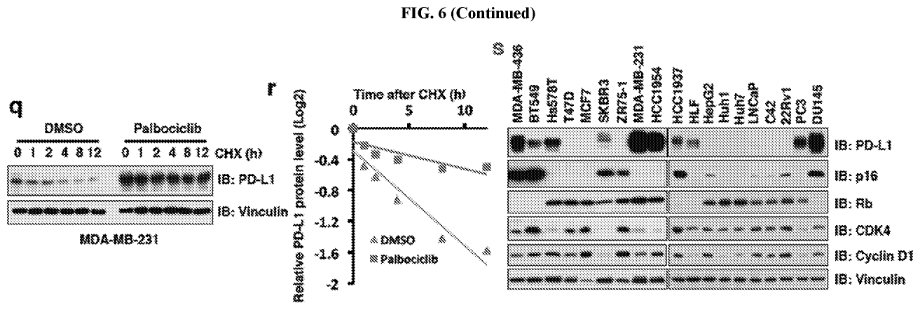

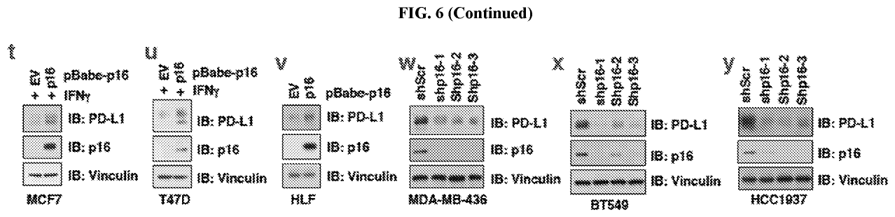

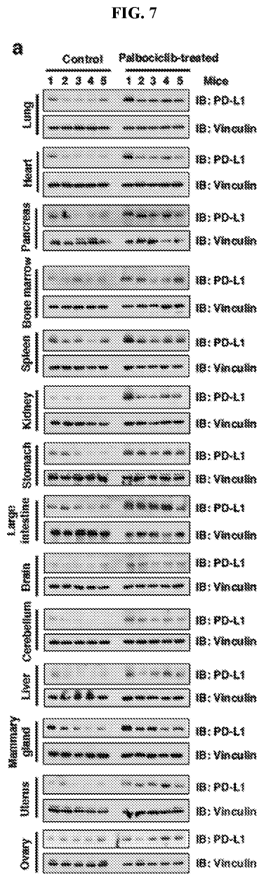

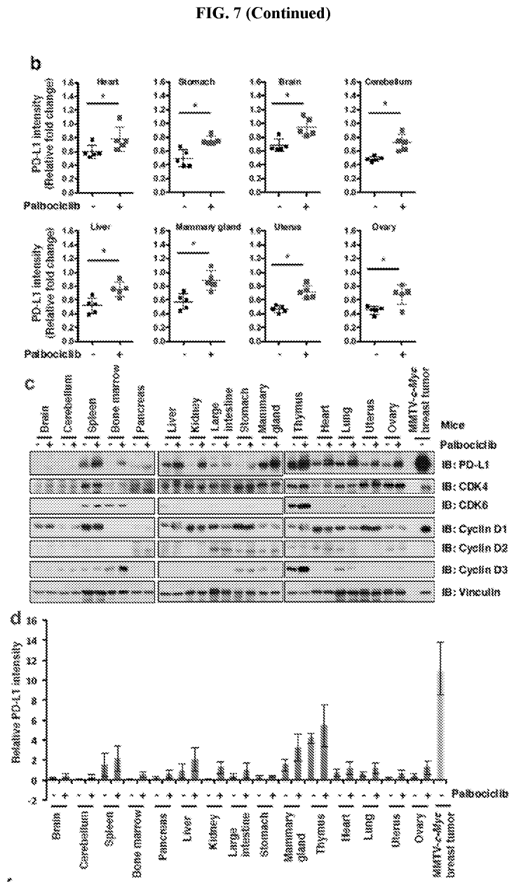

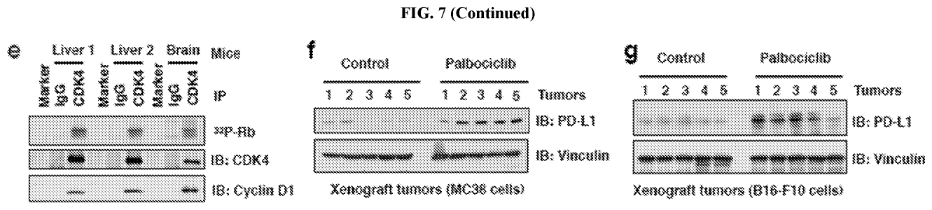

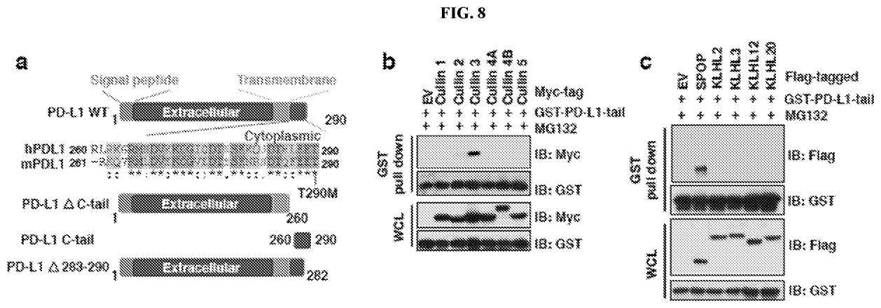

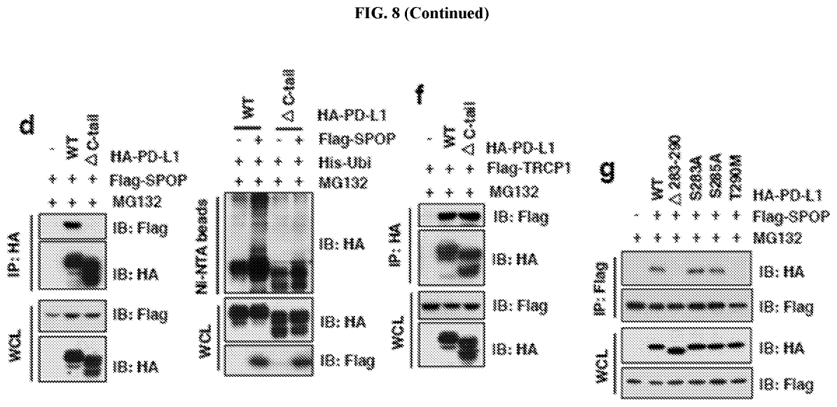

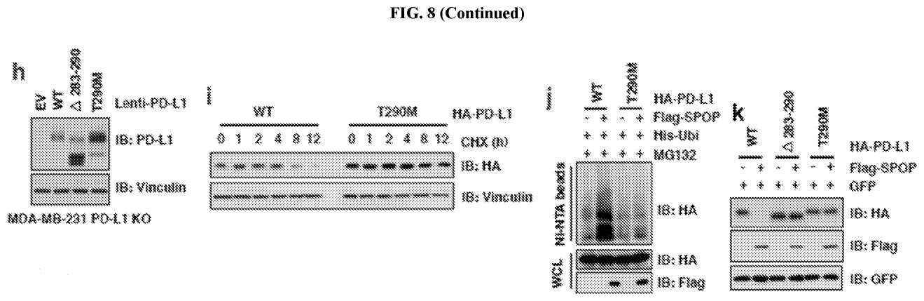

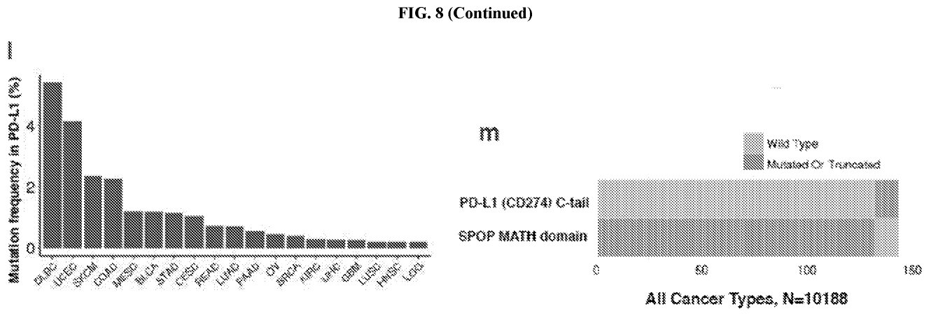

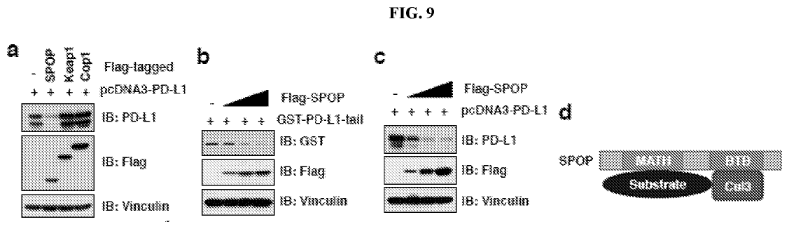

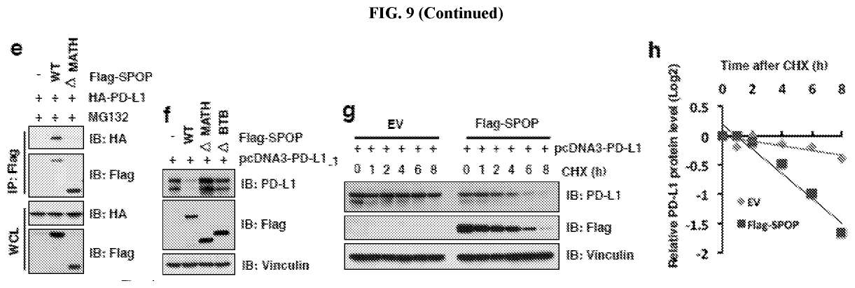

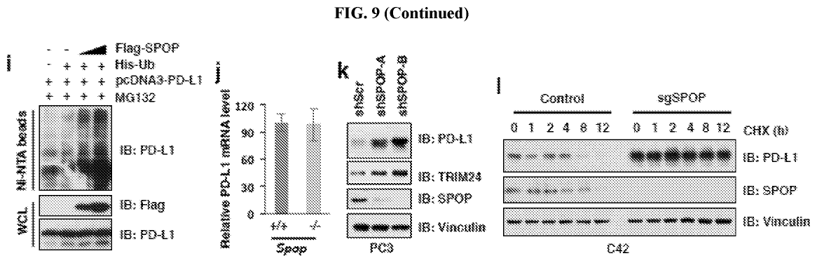

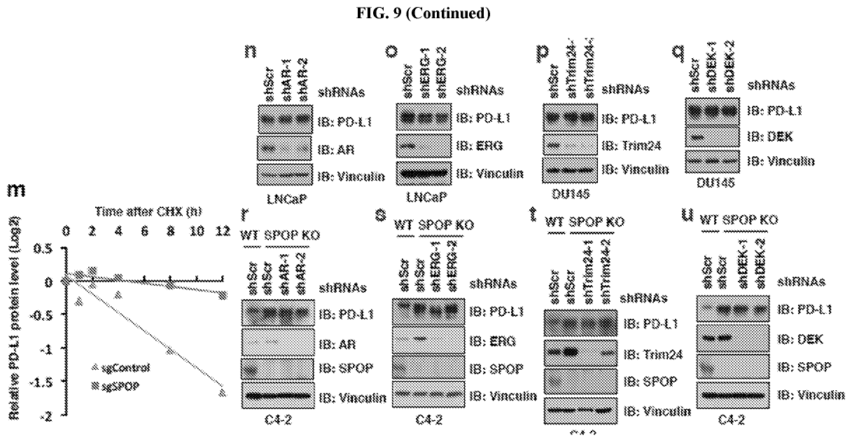

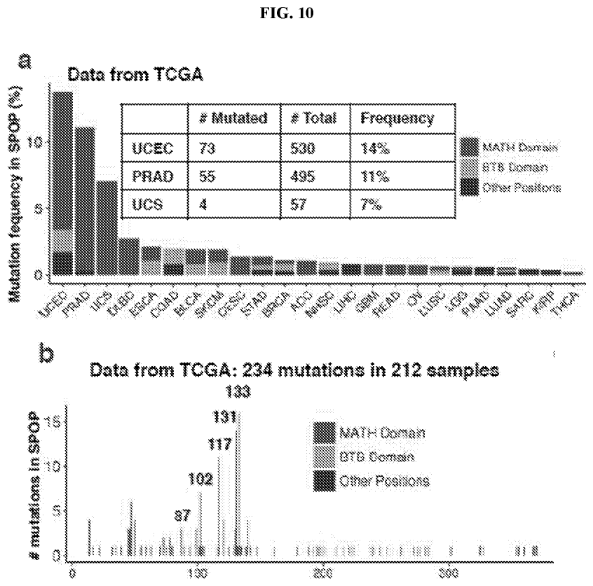

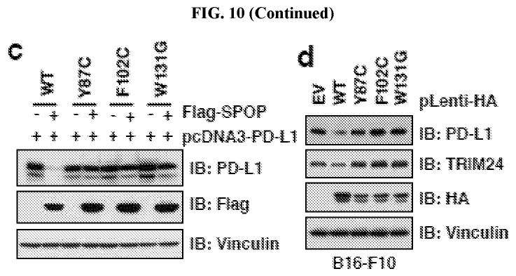

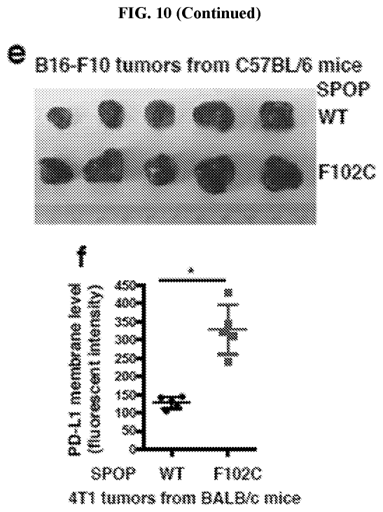

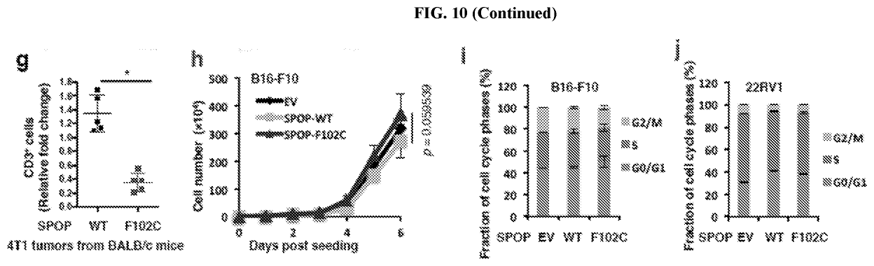

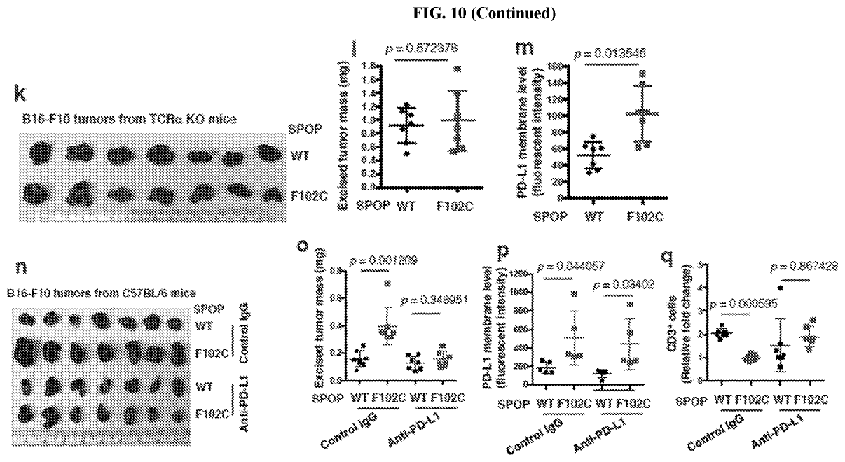

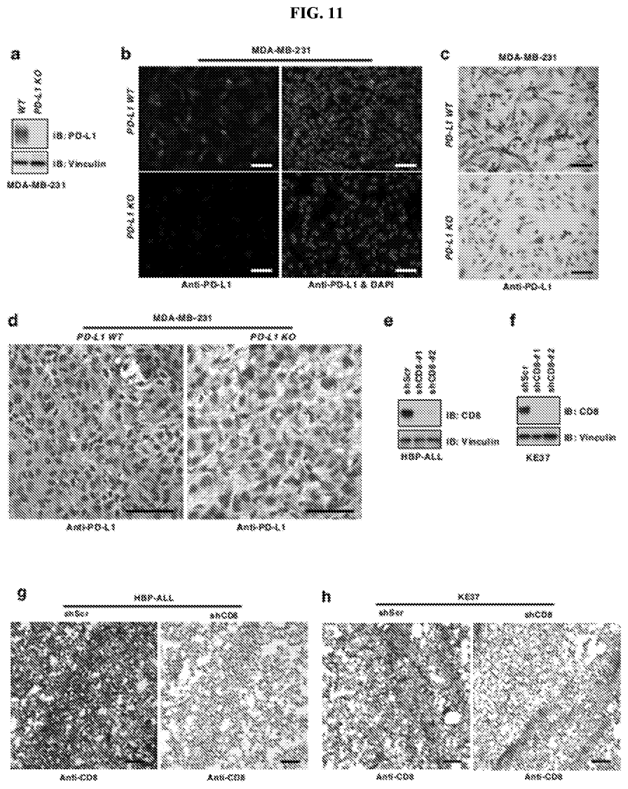

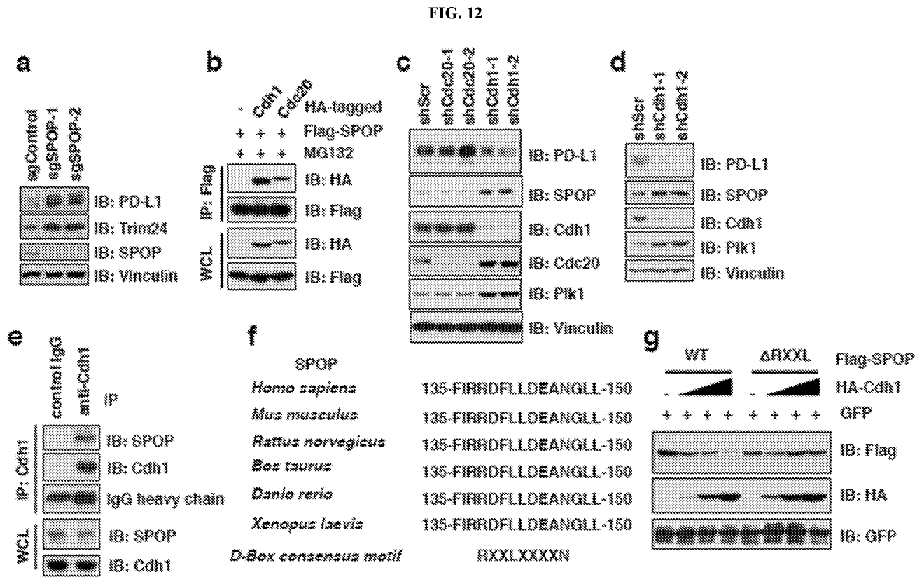

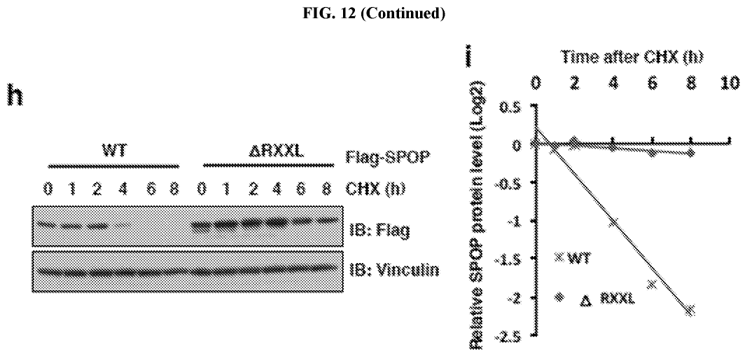

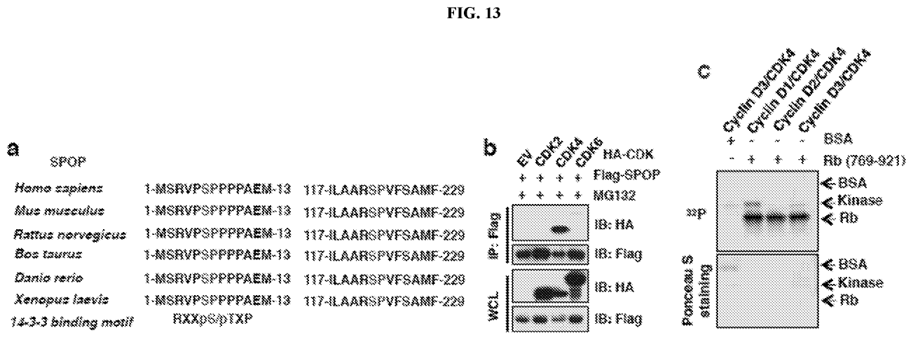

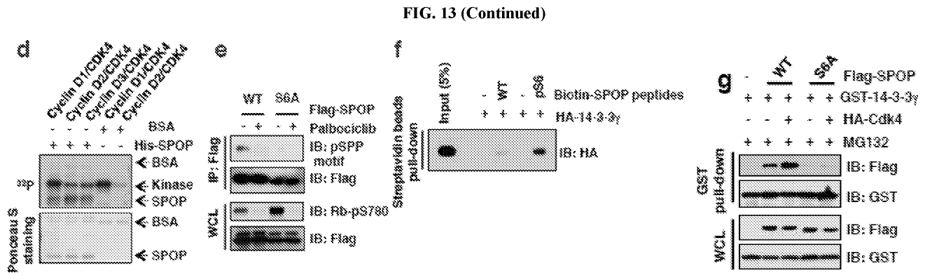

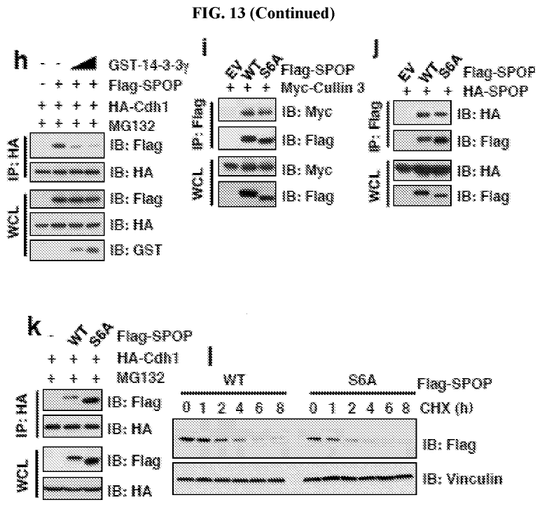

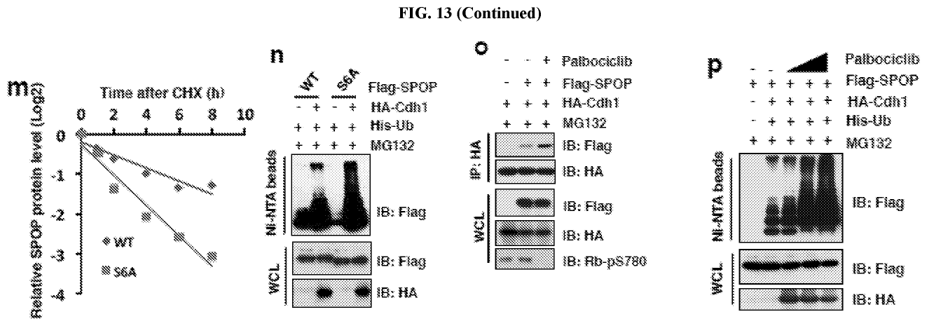

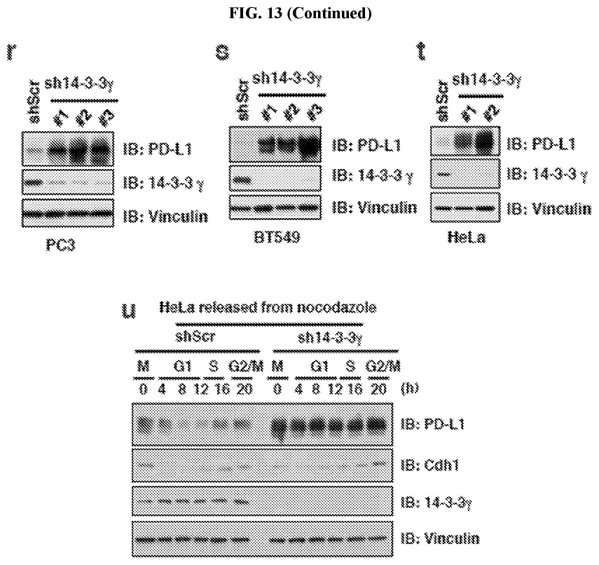

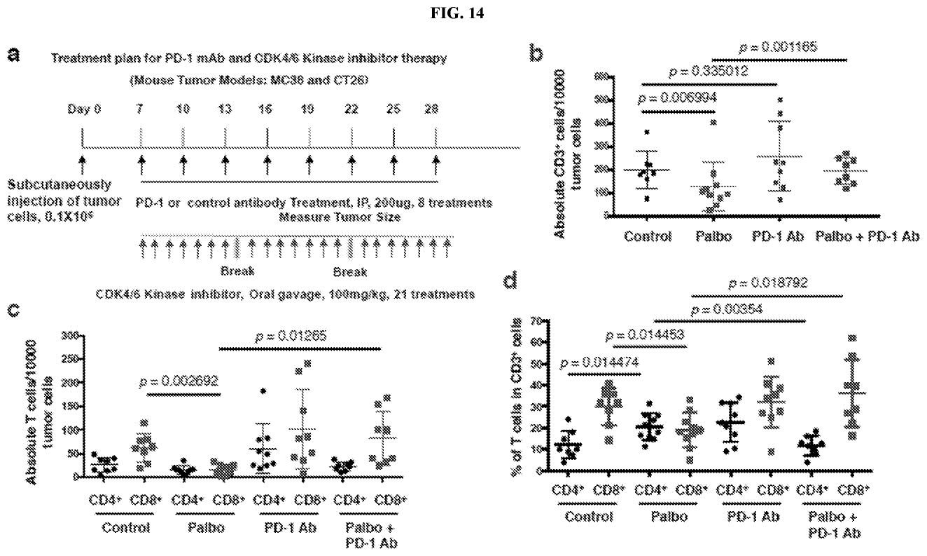

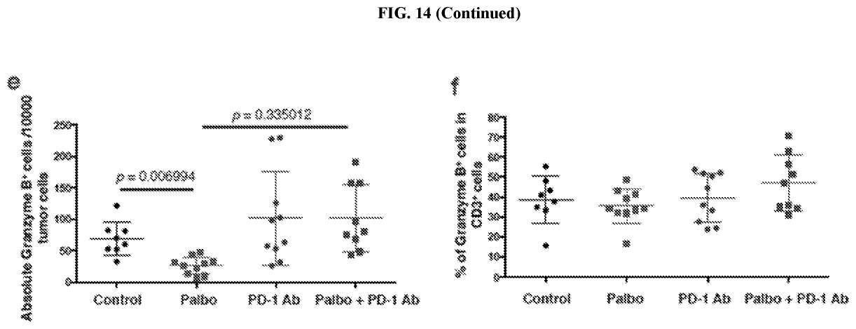

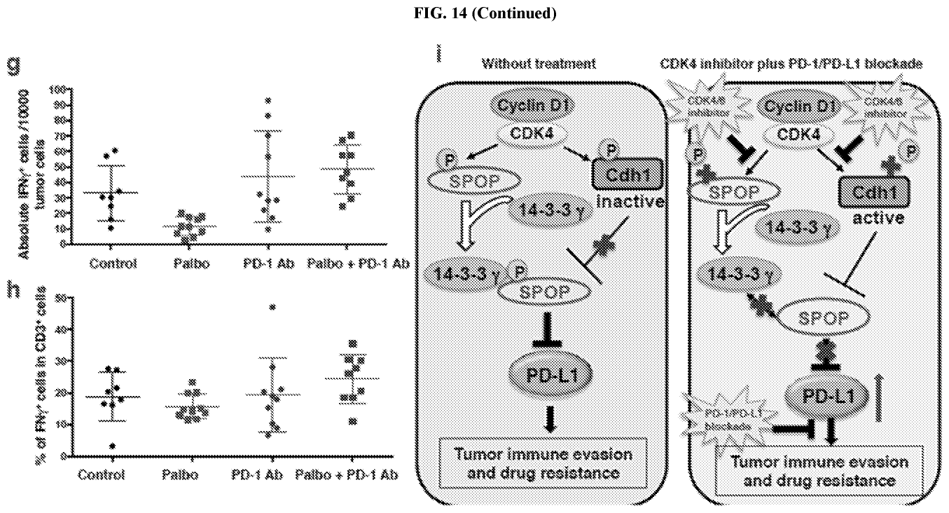

IB of WCL derived from HeLa cells pre-treated with/without IFNγ (10 ng/ml) for 12 hours and then synchronized in M phase by nocodazole treatment prior to releasing back into the cell cycle for the indicated times. FIG. 5F shows an IB of WCL derived from HeLa cells stably expressing HA-c-Myc WT, or HA-T58A/S62A-c-Myc as well as empty vector (EV) as a negative control. FIG. 5G shows an IB of WCL derived from HeLa cells with/without stably expressing HA-c-Myc WT synchronized in M phase by nocodazole treatment prior to releasing back into the cell cycle for the indicated times. FIG. 5H and FIG. 5I show an IB of WCL derived from 4T1 or B16-F10 mouse tumor cells treated with the indicated concentration of nocodazole for 20 hours before harvesting.

FIG. 6A, FIG. 6B, FIG. 6C, FIG. 6D, FIG. 6E, FIG. 6F, FIG. 6G, FIG. 6H, FIG. 6I, FIG. 6J, FIG. 6K, FIG. 6L, FIG. 6M, FIG. 6N, FIG. 6O, FIG. 6P, FIG. 6Q, FIG. 6R, FIG. 6S, FIG. 6T, FIG. 6U, FIG. 6V, FIG. 6W, FIG. 6X, and FIG. 6Y are graphs and images showing that cyclin D/CDK4 negatively regulates PD-L1 protein stability. FIG. 6A and FIG. 6B show an immunoblot (IB) analysis of whole cell lysates (WCL) derived from wild type (WT), cyclin A1^{-/-}A2^{-/-} or WT, cyclin E1^{-/-}E2^{-/-} MEFs. FIG. 6C shows quantitative real-time PCR (qRT-PCR) analysis of relative mRNA levels of PD-L1 from wild type MEFs and cyclin D1^{-/-}D2^{-/-}D3^{-/-}MEFs. Data were represented as mean±S.D, n=5. FIG. 6D shows cell cycle profiles for WT and cyclin D1^{-/-}D2^{-/-}D3^{-/-}MEFs, which were labeled with BrdU and analyzed by FACS. FIG. 6E shows an IB analysis of WCL derived from cyclin D1^{fl/fl}D2^{-/-}D3^{fl/fl} MEFs with or without depleting cyclin D1 and cyclin D3 by pLenti-Cre via viral infection (pLenti-EGFP as a negative control), selected with puromycin (1 μg/ml) for 72 hours before harvesting. FIG. 6F shows an IB analysis of WCL derived from MDA-MB-231 cells infected with indicated lentiviral shRNAs against cyclin D1 and cyclin D3, and selected with puromycin (1 μg/ml) for 72 hours before harvesting. FIG. 6G shows an IB analysis of WCL derived from cyclin D1^{-/-}D2^{-/-}D3^{-/-}MEFs stably reintroducing cyclin D1, cyclin D2, or cyclin D3, respectively, with empty vector (EV) as a negative control. FIG. 6H and FIG. 6I show an IB analysis of WCL derived from MDA-MB-231 cells stably expressing shCDK4 or shCDK2 as well as shScr as a negative control, respectively. FIG. 6J shows an IB analysis of WCL derived from WCL derived from wild type and cdk6^{-/-}MEFs. FIG. 6K shows an IB analysis of WCL derived from MDA-MB-231 cells stably expressing shCDK6 as well as shScr as a negative control. FIG. 6L and FIG. 6M show an IB analysis of WCL derived from MDA-MB-231 cells transfected with indicated constructs and the intensity of PD-L1 band was quantified by the ImageJ software. FIG. 6N shows an IB analysis of WCL derived from MDA-MB-231 cells depleted of Rb (with shScr as a negative control) treated with the CDK4/6 inhibitor, palbociclib, where indicated. FIG. 6O and FIG. 6P show an IB analysis of WCL derived from mouse CT26 or 4T1 tumor cell lines treated with or without the CDK4/6 inhibitor, palbociclib or ribociclib, respectively. FIG. 6Q and FIG. 6R show an IB analysis of WCL derived from MDA-MB-231 cells pre-treated with palbociclib (1 μM) for 36 hours before treatment with cycloheximide (CHX) for the indicated time points and PD-L1 protein abundance was quantified by the ImageJ and plotted as indicated. FIG. 6S shows an IB analysis of WCL derived from 19 different cancer cell lines with indicated antibodies. FIG. 6T, FIG. 6U, and FIG. 6V show an IB analysis of WCL derived from MCF7, T47D or HLF stably expressing p16 as well as EV as a negative control. FIG. 6W, FIG. 6X, and FIG. 6Y show an IB analysis of WCL derived from MDA-MB-436, BT549 or HCC1937 stably expressing three independent shRNAs against p16 as well as shScr as a negative control.

FIG. 7A, FIG. 7B, FIG. 7C, FIG. 7D, FIG. 7E, FIG. 7F, and FIG. 7G are graphs and images showing that treatment with the CDK4/6 inhibitor, palbociclib, elevated PD-L1 levels in vivo. FIG. 7A shows an immunoblot (IB) analysis of whole cell lysates (WCL) derived from multiple organs in mice treated with palbociclib (150 mg/kg body weight, by gastric gavage) or vehicle for 7 days. 5 mice per experimental group. FIG. 7B shows quantification of PD-L1 protein bands intensity in FIG. 7A by using the ImageJ software. 5 mice per experimental group. FIG. 7C shows IB analysis of WCL derived from 15 different tissues with/without palbociclib treatment and MMTV-c-Myc induced breast tumors. FIG. 7D shows quantification of PD-L1 protein bands intensity in FIG. 7C by using the ImageJ software. 3 mice per experimental group. FIG. 7E shows an in vitro kinase assay for Rb through using immunoprecipitated CDK4/cyclin D kinase complex from liver or brain by anti-CDK4 antibody IP. FIG. 7F and FIG. 7G show IB analysis of WCL derived from MC38 or B16-F10 mouse tumor cell line xenografted tumors treated with palbociclib (150 mg/kg body weight, by gastric gavage) or vehicle for 7 days. 5 mice per experimental group.

FIG. 8A, FIG. 8B, FIG. 8C, FIG. 8D, FIG. 8E, FIG. 8F, FIG. 8G, FIG. 8H, FIG. 8I, FIG. 8J, FIG. 8K, FIG. 8L, and FIG. 8M are graphs and images showing that Cullin 3^{SPOP} promotes PD-L1 ubiquitination and subsequent degradation largely through interaction with the cytoplasmic tail of PD-L1. FIG. 8A shows a schematic illustration of PD-L1 with N-terminal signal peptide, extracellular domain, trans-membrane domain, cytoplasmic tail and the potential SPOP-binding motif in PD-L1. FIG. 8B and FIG. 8C show immunoblot (IB) analyses of whole cell lysates (WCL) and GST pull-down precipitates derived from 293T cells transfected with indicated constructs and treated with MG132 (10 μM) for 12 hours before harvesting. FIG. 8D and FIG. 8F shows an IB analysis of WCL and immunoprecipitation (IP) derived from 293T cells transfected with indicated constructs and treated with MG132 (10 μM) for 12 hours before harvesting. FIG. 8E shows an IB of WCL and Ni-NTA pull-down products derived from the lysates of PC3 cells transfected with the indicated constructs. Cells were treated with MG132 (30 μM) for 6 hours before harvesting and lysed in the denature buffer. FIG. 8G shows an IB analysis of WCL and IP derived from 293T cells transfected with indicated constructs and treated with MG132 (10 μM) for 12 hours before harvesting. FIG. 8H shows an IB of WCL derived from MDA-MB-231 PD-L1 KO cells stably expressing PD-L1 WT, delta 283-290, T290M as well as EV as a negative control. FIG. 8I shows an IB analysis of WCL derived from 293T cells transfected with HA-PD-L1 WT and the T290M mutant, which were treated with cycloheximide (CHX) for indicated time points before harvesting. FIG. 8J shows an IB of WCL and Ni-NTA pull-down products derived from the lysates of PC3 cells transfected with the indicated constructs. Cells were treated with MG132 (30 μM) for 6 hours before harvesting and lysed in the denaturing buffer. FIG. 8K shows an IB of WCL derived from 293T cells transfected with indicated constructs. FIG. 8L shows the mutation frequency (mutated cases/total cases) of PD-L1 (CD274) across 19 cancer types from the TCGA database. FIG. 8M shows an oncoplot of PD-L1 (CD274) and SPOP across all 39 cancer types in the TCGA database. Only mutations or truncations in the C terminal tail of PD-L1 or in the MATH domain of SPOP are counted.

FIG. 9A, FIG. 9B, FIG. 9C, FIG. 9D, FIG. 9E, FIG. 9F, FIG. 9G, FIG. 9H, FIG. 9I, FIG. 9J, FIG. 9K, FIG. 9L, FIG. 9M, FIG. 9N, FIG. 9O, FIG. 9P, FIG. 9Q, FIG. 9R, FIG. 9S, FIG. 9T, and FIG. 9U are graphs and images showing that SPOP negatively regulates PD-L1 protein stability in a poly-ubiquitination dependent manner. FIG. 9A, FIG. 9B, and FIG. 9C show immunoblot (IB) analysis of whole cell lysates (WCL) derived from 293T cells transfected with indicated constructs. FIG. 9D shows a schematic illustration of SPOP with MATH and BTB domain to interact with substrate and Cullin 3, respectively. FIG. 9E shows an IB analysis of WCL and IP derived from 293T cells transfected with indicated constructs and treated with MG132 (10 μM) for 12 hours before harvesting. FIG. 9F shows an IB analysis of WCL derived from 293T cells transfected with indicated constructs. FIG. 9G shows an IB analysis of WCL derived from 293T cells transfected with indicated constructs. 36 h post transfection, cells were treated with 20 μg/ml cycloheximide (CHX) at indicated time points. The PD-L1 protein abundance were quantified by the ImageJ software and plotted in FIG. 9H. FIG. 9I shows an IB of WCL and Ni-NTA pull-down products derived from the lysates of PC3 cells transfected with the indicated constructs. Cells were treated with MG132 (30 μM) for 6 hours before harvesting and lysed in the denaturing buffer. FIG. 9J shows qRT-PCR analysis of relative mRNA levels of PD-L1 from Spop^{+/+} and Spop^{-/-} MEFs. Data were represented as mean±S.D, n=5. FIG. 9K shows IB analysis of WCL derived from PC3 cells infected with indicated lentiviral shRNAs against SPOP and selected with puromycin (1 μg/ml) for 72 hours before harvesting. FIG. 9L shows an IB analysis of WCL derived from C42 cells with depletion of SPOP using sgRNA and treated with cycloheximide (CHX) for indicated time points before harvesting. The PD-L1 protein abundance were quantified by the ImageJ software and plotted (FIG. 9M). FIG. 9N and FIG. 90 show IB analysis of WCL derived from LNCap cells stably expressing shAR or shERG as well as shScr as a negative control. FIG. 9P and FIG. 9Q show IB analysis of WCL derived from DU145 cells stably expressing shTrim24 or shDEK as well as shScr as a negative control. FIG. 9R, FIG. 9S, FIG. 9T, and FIG. 9U show IB analysis of WCL derived from C42 WT and SPOP KO cells that stably expressed shAR, shERG, shTrim24, or shDEK as well as shScr, respectively.

FIG. 10A, FIG. 10B, FIG. 10C, FIG. 10D, FIG. 10E, FIG. 10F, FIG. 10G, FIG. 10H, FIG. 10I, FIG. 10J, FIG. 10K, FIG. 10L, FIG. 10M, FIG. 10N, FIG. 10O, FIG. 10P, and FIG. 10Q are graphs and images showing that cancer-derived SPOP mutations fail to promote PD-L1 degradation. FIG. 10A is a graph showing the mutation frequency (mutated cases/total cases) of SPOP across 24 cancer types from the TCGA database. Mutations are categorized as happening in the MATH domain, in the BTB domain or at any other position of the gene, including UTRs. Because some patient cases contain mutations of two or three categories, the proportions of three colors are allocated mutation-wise, instead of case-wise. FIG. 10B shows the distribution of mutation positions of SPOP in 24 cancer types from the TCGA database. Mutations with low translational consequences have been discarded. FIG. 10C shows an immunoblot (IB) analysis of whole cell lysates (WCL) derived from 293T cells transfected with indicated constructs. FIG. 10D shows an IB of WCL derived from B16-F10 mouse tumor cell line stably expressing the indicated SPOP constructs. FIG. 10E shows B16-F10 cells implanted tumors from C57BL/6 mice were dissected and taken a picture after euthanizing the mice. FIG. 10F and FIG. 10G show the relative cell surface PD-L1 expression and CD3⁺ T-cell populations from the isolated tumor-infiltrating lymphocytes in 4T1 xenografted tumors ectopically expressing SPOP-WT or the SPOP-F102C mutant; samples were subjected to FACS analysis. Data were represented as mean±S.E., N=5, and *p<0.05 (Student's t-test). FIG. 10H and FIG. 10I show growth curve and cell cycle profiles of B16-F10 cells stably expressing SPOP WT and the F102C mutant as well as EV as a negative control. FIG. 10J shows a cell cycle profile of 22RV1 cells stably expressing SPOP WT and the F102C mutant as well as EV as a negative control. FIG. 10K is an image showing B16-F10 cells implanted tumors from TCRα KO mice were dissected and taken a picture after euthanizing the mice. FIG. 10L and FIG. 10M show the relative cell surface PD-L1 expression and CD3⁺ T-cell populations from the isolated tumor-infiltrating lymphocytes in B16-F10 cells implanted tumors ectopically expressing SPOP-WT or the SPOP-F102C mutant were subjected to FACS analysis. Data were represented as mean±S.E., N=5, and *p<0.05 (Student's t-test). FIG. 10N is an image showing B16-F10 cells implanted tumors from C57BL/6 mice treated with control IgG or anti-PD-L1 antibody were dissected and taken a picture after euthanizing the mice. FIG. 10O shows the weight of B16-F10 cells implanted tumors from C57BL/6 mice treated with control IgG or anti-PD-L1 antibody. Data were represented as mean±S.E., N=7, and *p<0.05 (Student's t-test). FIG. 10P and FIG. 10Q show the relative cell surface PD-L1 expression and CD3⁺ T-cell populations from the isolated tumor-infiltrating lymphocytes in B16-F10 cells implanted tumors ectopically expressing SPOP-WT or the SPOP-F102C mutant treated with control IgG or anti-PD-L1 antibody were subjected to FACS analysis. Data were represented as mean±S.E., N=7, and *p<0.05 (Student's t-test).

FIG. 11A, FIG. 11B, FIG. 11C, FIG. 11D, FIG. 11E, FIG. 11F, FIG. 11G and FIG. 11H are images showing the validation of anti-PD-L1 and anti-CD8 antibodies through using PD-L1 KO or shCD8 cells. FIG. 11A shows an immunoblot (IB) analysis of whole cell lysates (WCL) derived from MDA-MB-231 cells depleted PD-L1 through the CRISPR-Cas9 system. FIG. 11B and FIG. 11C show an immunostaining for MDA-MB-231 PD-L1 WT and KO cells using the anti-PD-L1 antibody. FIG. 11D shows an immunochemistry (IHC) image of MDA-MB-231 PD-L1 WT and KO cells using the anti-PD-L1 antibody. FIG. 11E shows an IB analysis of WCL derived from HBP-ALL cells stably expressing shCD8 as well as shScr as a negative control using the anti-CD8 antibody. FIG. 11F shows an IB analysis of WCL derived from KE37 cells stably expressing shCD8 as well as shScr as a negative control using the anti-CD8 antibody. FIG. 11G shows an immunochemistry (IHC) image of HBP-ALL cell pellets stably expressing shCD8 as well as shScr as a negative control using the anti-CD8 antibody. The scale bar represents 50 μm. FIG. 11H shows an immunochemistry (IHC) image of KE37 cell pellets stably expressing shCD8 as well as shScr as a negative control using the anti-CD8 antibody. The scale bar represents 50 μm.

FIG. 12A, FIG. 12B, FIG. 12C, FIG. 12D, FIG. 12E, FIG. 12F, FIG. 12G, FIG. 12H, FIG. 12I are graphs and images showing that depletion of Cdh1, but not Cdc20, prolongs SPOP proteins stability, which is simultaneously coupled with a decrease in PD-L1 protein level. FIG. 12A shows an immunoblot (IB) analysis of whole cell lysates (WCL) derived from HeLa depleted SPOP through the CRISPR-Cas9 system. FIG. 12B shows an IB analysis of WCL and immunoprecipitation (IP) derived from 293T cells transfected with indicated constructs and treated with MG132 (10 μM) for 12 hours before harvesting. FIG. 12C and FIG. 12D IB show an analysis of WCL derived from HeLa depleted Cdc20 or Cdh1 through multiple independent shRNAs. FIG. 12E shows an IB analysis of WCL and IP derived from HeLa cells treated with MG132 (10 μM) for 12 hours before harvesting. FIG. 12F shows a sequence comparison of D-box motif (RxxLxxxxN) in SPOP derived from different species. FIG. 12G shows an IB analysis of WCL derived from HeLa cells transfected with indicated constructs. FIG. 12H and FIG. 12I IB show an analysis of WCL derived from 293T cells transfected with indicated constructs. 36 h post transfection, cells were treated with 20 μg/ml cycloheximide (CHX) as indicated time points before harvesting. The protein abundance of SPOP-WT and deletion of RxxL mutant were quantified by the ImageJ software.

FIG. 13A, FIG. 13B, FIG. 13C, FIG. 13D, FIG. 13E, FIG. 13F, FIG. 13G, FIG. 13H, FIG. 13I, FIG. 13J, FIG. 13K, FIG. 13L, FIG. 13M, FIG. 13N, FIG. 13O, FIG. 13P, FIG. 13R, FIG. 13S, FIG. 13T, and FIG. 13U are graphs and images showing cyclin D/CDK4-mediated phosphorylation of SPOP at the Ser6 residue promotes its binding with 14-3-3γ to reduce its poly-ubiquitination and subsequent degradation by APC/Cdh1. FIG. 13A shows a sequence comparison of conserved SP sites and putative 14-3-3γ binding motif in SPOP. FIG. 13B is an immunoblot (IB) analysis of whole cell lysates (WCL) and immunoprecipitation (IP) derived from 293T cells transfected with indicated constructs and treated with MG132 (10 tM) for 12 hours before harvesting. FIG. 13C, FIG. 13D show in vitro kinase assays with recombinant Rb and SPOP as substrates and cyclin D1/CDK4, cyclin D2/CDK4 and cyclin D3/CDK4 as kinase complex were performed. BSA was used as a negative control where indicated. FIG. 13E shows an IB analysis of WCL and immunoprecipitation (IP) derived from MDA-MB-231 cells transfected with indicated constructs, which were treated with/without palbociclib (1 μM) for 12 hours. FIG. 13F shows a streptavidin beads pull-down assay for biotin-labeled SPOP peptide with/without phosphorylation at the Ser6 residue to examine its in vitro association with 14-3-3γ. FIG. 13G shows an IB analysis of WCL and GST pull-down precipitates derived from 293T cells transfected with indicated constructs and treated with MG132 (10 tM) for 12 hours before harvesting. FIG. 13H, FIG. 13I, FIG. 13J, and FIG. 13K show an IB analysis of WCL and IP derived from 293T cells transfected with indicated constructs and treated with MG132 (10 tM) for 12 hours before harvesting. FIG. 13L an FIG. 13M show an IB analysis of WCL derived from 293T cells transfected with indicated constructs. 36 h post transfection, cells were treated with 20 μg/ml cycloheximide (CHX) as indicated time points. The protein abundance of SPOP-WT and S6A mutant were quantified by the ImageJ software and plotted accordingly. FIG. 13N shows an IB analysis of WCL and IP derived from 293T cells transfected with indicated constructs and treated with MG132 (10 tM) and with/without palbociclib (1 μM) for 12 hours before harvesting. FIG. 13O and FIG. 13P show an IB of WCL and Ni-NTA pull-down products derived from the lysates of PC3 cells transfected with the indicated constructs. Cells were treated with MG132 (30 tM) for 6 hours before harvesting and lysed in the denaturing buffer for following assays. FIG. 13R, FIG. 13S and FIG. 13T show an IB of WCLs derived from PC3, BT549 and HeLa cells stably expressing sh14-3-3γ as well as shScr as a negative control. FIG. 13U shows an IB of WCL derived from HeLa cells stably expressing shScr or sh/4-3-3γ synchronized in M phase by nocodazole treatment prior to releasing back into the cell cycle for the indicated times. FIG. 13Q has been intentionally omitted from the drawings.

FIG. 14A, FIG. 14B, FIG. 14C, FIG. 14D, FIG. 14E, FIG. 14F, FIG. 14G, FIG. 14H, FIG. 14I are graphs and images showing combination therapy of anti-PD-1 mAb and CDK4/6 inhibitor in MC38 colon cancer mouse model. FIG. 14A shows a schematic model that illustrates the treatment plan for mice bearing subcutaneous MC38 tumors. Female C57BL/6 mice were implanted with 0.1×10⁶ MC38 cells subcutaneously and treated with four arms: control antibody treatment, anti-PD-1 mAb treatment, CDK4/6 inhibitor treatment, anti-PD-1 mAb plus CDK4/6 inhibitor combination treatment. FIG. 14B, FIG. 14C and FIG. 14D show that a single agent CDK4/6 inhibitor palbociclib treatment significantly reduced the absolute number of CD3⁺ and CD8⁺ TILs as well as the frequency of CD8⁺ cells in CD3⁺ cell populations, which could be further rescued by combination of CDK4/6 inhibitor palbociclib plus anti-PD1 treatment. FIG. 14E, FIG. 14F, FIG. 14G and FIG. 14H show that the absolute number or Granzyme B+ and IFNγ⁺ of CD3⁺ cells were also reduced after CDK4/6 inhibitor treatment, which could be recovered by combination of CDK4/6 inhibitor palbociclib plus anti-PD1 treatment. FIG. 14I shows a proposed working model to illustrate how PD-L1 protein stability is regulated by the cyclin D/CDK4-SPOP-Cdh1 signaling pathway. The cyclin D/CDK4 negatively regulates PD-L1 protein stability largely through phosphorylating its upstream physiological E3 ligase SPOP to promote SPOP binding with 14-3-3γ, which subsequently disrupts Cdh1-mediated destruction of SPOP. As such, CDK4/6 inhibitor treatment could unexpectedly elevate PD-L1 protein levels largely through inhibiting cyclin D/CDK4-mediated phosphorylation of SPOP to promote its degradation by APC/C^{Cdh1}. The unexpected rise of PD-L1 could present a severe clinical problem for patients receiving CDK4 inhibitor treatment and could be one of the underlying mechanisms accounting for CDK4 inhibitor resistance via evading immune surveillance checkpoint. Hence, our work provides a novel molecular mechanism as well as the rationale for the combinational treatment of PD-L1 blockage treatment and the CDK4/6 inhibitors as a more efficient anti-cancer clinical option.

### DETAILED DESCRIPTION OF THE INVENTION

The present disclosure features compositions and methods of treating a cancer in a subject by administering to the subject an anti-PD-L1 or an anti-PD-1 antibody and an inhibitor of cyclin D kinase (CDK)4/6 to treat the cancer.

The invention is based, at least in part, on the discovery that PD-L1 protein abundance fluctuates during the cell cycle and is negatively regulated during cell cycle progression by cyclin D/CDK4 and the Cullin 3SPOP E3 ligase. This fluctuation is mediated by proteasome degradation. Treatment with an inhibitor of CDK4/6, Palbociclib, elevated PD-L1 protein levels largely through inhibiting cyclin D/CDK4-mediated phosphorylation of SPOP to promote its degradation by APC/Ccdh1. Loss-of-function mutations in SPOP compromised ubiquitination-mediated PD-L1 degradation, and led to increased PD-L1 expression and reduced tumor-infiltrating lymphocytes (TILs) in mouse tumors and human prostate cancer specimens. Upregulation of PD-L1 by CDK4/6 inhibition helped tumors evade immune surveillance, which could be one possible underlying reason for acquired drug resistance to CDK4/6 inhibitors in clinical therapy. Combining CDK4/6 inhibitor treatment with anti-PD-1 immunotherapy enhanced tumor regression and dramatically improved overall survival rates in mouse tumor models. The present invention uncovers a novel molecular mechanism for regulating PD-L1 protein stability during cell cycle progression, and shows that using combination treatment with CDK4/6 inhibitors and PD-1/PD-L1 immune checkpoint blockade enhances the therapeutic efficacy for human cancers.

### Therapeutic Combinations of the Invention

The invention provides compositions comprising a CDK4/6 inhibitor and an anti-PD-1 and/or anti-PD-L1 antibody.

Antibodies that target PD-1 include, but are not limited to, nivolumab, Bristol-Myers Squibb; pembrolizumab, Merck, Whitehouse Station, N.J.; pidilizumab, CureTech, Yavne, Israel).

Antibodies that target PD-L1 include, but are not limited to MPDL3280A, Genentech, South San Francisco, Calif.; MEDI4736, MedImmune/AstraZeneca; BMS-936559, Bristol-Myers Squibb; MSB0010718C, EMD Serono, Rockland, Mass.).

Examples of CDK4/6 inhibitors include palbociclib (PD0332991), ribociclib (LEE011), abemaciclib (LY2835219) and trilaciclib (G1T28).

### Therapeutic Methods

The methods and compositions provided herein can be used to treat or prevent progression of a cancer (e.g., colon cancer, breast cancer, melanoma, prostate cancer, lung cancer, head and neck cancer). In general, methods of the invention involve the administration of therapeutic combinations comprising an agent that inhibits the expression or activity of cyclin-dependent kinase 4/6 (CDK4/6); and an agent that targets PD-L1 or programmed cell death-1 (PD-1) Compositions of the invention are administered to subjects, particularly humans, suffering from, having, susceptible to, or at risk of developing cancer (e.g., colon cancer, breast cancer, melanoma, prostate cancer, lung cancer, head and neck cancer). Determination of those subjects “at risk” can be made by any objective or subjective determination by a diagnostic test or opinion of a subject or health care provider (e.g., genetic test, enzyme or protein marker, family history, and the like). Identifying a subject in need of such treatment can be in the judgment of a subject or a health care professional and can be subjective (e.g. opinion) or objective (e.g., measurable by a test or diagnostic method).

In one embodiment, a therapeutic combination of the invention comprises a CDK4/6 inhibitor, an anti-PD-L1 antibody, and/or an anti-PD-1 antibody. In particular embodiments, a therapeutic combination of the invention comprises a CDK4/6 inhibitor and an anti-PD-L1 antibody or an anti-PD-1 antibody. The CDK4/6 inhibitor may be administered prior to, concurrent with, or subsequent to administration of the anti-PD-L1 antibody or an anti-PD-1 antibody. In particular embodiments, the CDK4/6 inhibitor and the anti-PD-L1 antibody and/or anti-PD-1 antibody administration is conducted within about 1-3 hours, 4-6 hours, 7-12 hours, or 13-24 hours. In other embodiments, the administration occurs within about 1-3 days, 3-5 days, or 7-10 days. If desired, such therapeutic combinations are administered in combination with standard chemotherapeutics. Methods for administering combination therapies (e.g., concurrently or otherwise) are known to the skilled artisan and are described for example in Remington's Pharmaceutical Sciences by E. W. Martin.

### Pharmaceutical Compositions

The present invention features compositions comprising an agent that inhibits the function, expression or activity of cyclin-dependent kinase 4/6 (CDK4/6); and an agent that targets PD-L1 or programmed cell death-1 (PD-1), which are useful for treating cancer (e.g., Typically, such compositions comprise an effective amount of an agent that inhibits the expression or activity of cyclin-dependent kinase 4/6 (CDK4/6); and an effective amount of an agent that targets PD-L1 or programmed cell death-1 (PD-1) in a physiologically acceptable carrier. Therapeutic combinations of the invention are typically formulated and administered separately, but may also be combined and administered in a single formulation.

Typically, the carrier or excipient for the composition provided herein is a pharmaceutically acceptable carrier or excipient, such as sterile water, aqueous saline solution, aqueous buffered saline solutions, aqueous dextrose solutions, aqueous glycerol solutions, ethanol, or combinations thereof. The preparation of such solutions ensuring sterility, pH, isotonicity, and stability is effected according to protocols established in the art. Generally, a carrier or excipient is selected to minimize allergic and other undesirable effects, and to suit the particular route of administration, e.g., subcutaneous, intramuscular, intranasal, and the like.

The administration may be by any suitable means that results in a concentration of the therapeutic that, combined with other components, is effective in ameliorating, reducing, or stabilizing the disease symptoms in a subject. The composition may be administered systemically, for example, formulated in a pharmaceutically-acceptable buffer such as physiological saline. Preferable routes of administration include, for example, subcutaneous, intravenous, intraperitoneally, intramuscular, intrathecal, or intradermal injections that provide continuous, sustained levels of the agent in the patient. The amount of the therapeutic agent to be administered varies depending upon the manner of administration, the age and body weight of the patient, and with the clinical symptoms of the cancer. Generally, amounts will be in the range of those used for other agents used in the treatment of cancer, although in certain instances lower amounts will be needed because of the increased specificity of the agent. A composition is administered at a dosage that ameliorates or decreases effects of the cancer as determined by a method known to one skilled in the art.

The therapeutic or prophylactic composition may be contained in any appropriate amount in any suitable carrier substance, and is generally present in an amount of 1-95% by weight of the total weight of the composition. The composition may be provided in a dosage form that is suitable for parenteral (e.g., subcutaneously, intravenously, intramuscularly, intrathecally, or intraperitoneally) administration route. The pharmaceutical compositions may be formulated according to conventional pharmaceutical practice (see, e.g., Remington: The Science and Practice of Pharmacy (20th ed.), ed. A. R. Gennaro, Lippincott Williams & Wilkins, 2000 and Encyclopedia of Pharmaceutical Technology, eds. J. Swarbrick and J. C. Boylan, 1988-1999, Marcel Dekker, New York).

Pharmaceutical compositions according to the invention may be formulated to release the active agent substantially immediately upon administration or at any predetermined time or time period after administration. The latter types of compositions are generally known as controlled release formulations, which include (i) formulations that create a substantially constant concentration of the drug within the body over an extended period of time; (ii) formulations that after a predetermined lag time create a substantially constant concentration of the drug within the body over an extended period of time; (iii) formulations that sustain action during a predetermined time period by maintaining a relatively, constant, effective level in the body with concomitant minimization of undesirable side effects associated with fluctuations in the plasma level of the active substance (sawtooth kinetic pattern); (iv) formulations that localize action by, e.g., spatial placement of a controlled release composition adjacent to or in contact with an organ, such as the heart; (v) formulations that allow for convenient dosing, such that doses are administered, for example, once every one or two weeks; and (vi) formulations that target a disease using carriers or chemical derivatives to deliver the therapeutic agent to a particular cell type. For some applications, controlled release formulations obviate the need for frequent dosing during the day to sustain the plasma level at a therapeutic level.

Any of a number of strategies can be pursued in order to obtain controlled release in which the rate of release outweighs the rate of metabolism of the agent in question. In one example, controlled release is obtained by appropriate selection of various formulation parameters and ingredients, including, e.g., various types of controlled release compositions and coatings. Thus, the therapeutic agent is formulated with appropriate excipients into a pharmaceutical composition that, upon administration, releases the therapeutic agent in a controlled manner. Examples include single or multiple unit tablet or capsule compositions, oil solutions, suspensions, emulsions, microcapsules, microspheres, molecular complexes, nanoparticles, patches, and liposomes.

The pharmaceutical composition may be administered parenterally by injection, infusion or implantation (subcutaneous, intravenous, intramuscular, intraperitoneal, intrathecal, or the like) in dosage forms, formulations, or via suitable delivery devices or implants containing conventional, non-toxic pharmaceutically acceptable carriers and adjuvants. The formulation and preparation of such compositions are well known to those skilled in the art of pharmaceutical formulation. Formulations can be found in Remington: The Science and Practice of Pharmacy, supra.

Compositions for parenteral use may be provided in unit dosage forms (e.g., in single-dose ampoules), or in vials containing several doses and in which a suitable preservative may be added (see below). The composition may be in the form of a solution, a suspension, an emulsion, an infusion device, or a delivery device for implantation, or it may be presented as a dry powder to be reconstituted with water or another suitable vehicle before use. Apart from the active agent that reduces or ameliorates a cardiac dysfunction or disease, the composition may include suitable parenterally acceptable carriers and/or excipients. The active therapeutic agent(s) (e.g., a cyclin-dependent kinase 4/6 (CDK4/6) inhibitor and anti-PD-L1 or PD-1 antibody described herein) may be incorporated into microspheres, microcapsules, nanoparticles, liposomes, or the like for controlled release. Furthermore, the composition may include suspending, solubilizing, stabilizing, pH-adjusting agents, tonicity adjusting agents, and/or dispersing, agents.

In some embodiments, the composition comprising the active therapeutic agent is formulated for intravenous delivery. As indicated above, the pharmaceutical compositions according to the invention may be in the form suitable for sterile injection. To prepare such a composition, the suitable therapeutic(s) are dissolved or suspended in a parenterally acceptable liquid vehicle. Among acceptable vehicles and solvents that may be employed are water, water adjusted to a suitable pH by addition of an appropriate amount of hydrochloric acid, sodium hydroxide or a suitable buffer, 1,3-butanediol, Ringer's solution, and isotonic sodium chloride solution and dextrose solution. The aqueous formulation may also contain one or more preservatives (e.g., methyl, ethyl or n-propyl p-hydroxybenzoate). In cases where one of the agents is only sparingly or slightly soluble in water, a dissolution enhancing or solubilizing agent can be added, or the solvent may include 10-60% w/w of propylene glycol or the like.

### Kits

The invention provides kits for the treatment or prevention of cancer. In some embodiments, the kit includes a therapeutic composition containing an agent that inhibits the expression or activity of cyclin-dependent kinase 4/6 (CDK4/6); and an agent that targets PD-L1 or programmed cell death-1 (PD-1) in unit dosage form. In other embodiments, the kit includes an agent that inhibits the expression or activity of cyclin-dependent kinase 4/6 (CDK4/6); and an agent that targets PD-L1 or programmed cell death-1 (PD-1) in unit dosage form in a sterile container. Such containers can be boxes, ampoules, bottles, vials, tubes, bags, pouches, blister-packs, or other suitable container forms known in the art. Such containers can be made of plastic, glass, laminated paper, metal foil, or other materials suitable for holding medicaments.

If desired a pharmaceutical composition of the invention is provided together with instructions for administering the pharmaceutical composition to a subject having or at risk of contracting or developing cancer. The instructions will generally include information about the use of the composition for the treatment or prevention of cancer. In other embodiments, the instructions include at least one of the following: description of the therapeutic/prophylactic agent; dosage schedule and administration for treatment or prevention of cancer or symptoms thereof; precautions; warnings; indications; counter-indications; over dosage information; adverse reactions; animal pharmacology; clinical studies; and/or references. The instructions may be printed directly on the container (when present), or as a label applied to the container, or as a separate sheet, pamphlet, card, or folder supplied in or with the container.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987);

“Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

The following examples are put forth so as to provide those of ordinary skill in the art with a complete disclosure and description of how to make and use the assay, screening, and therapeutic methods of the invention, and are not intended to limit the scope of what the inventors regard as their invention.

### EXAMPLES

### Example 1: Protein Abundance of PD-L1 Fluctuates During Cell Cycle Progression

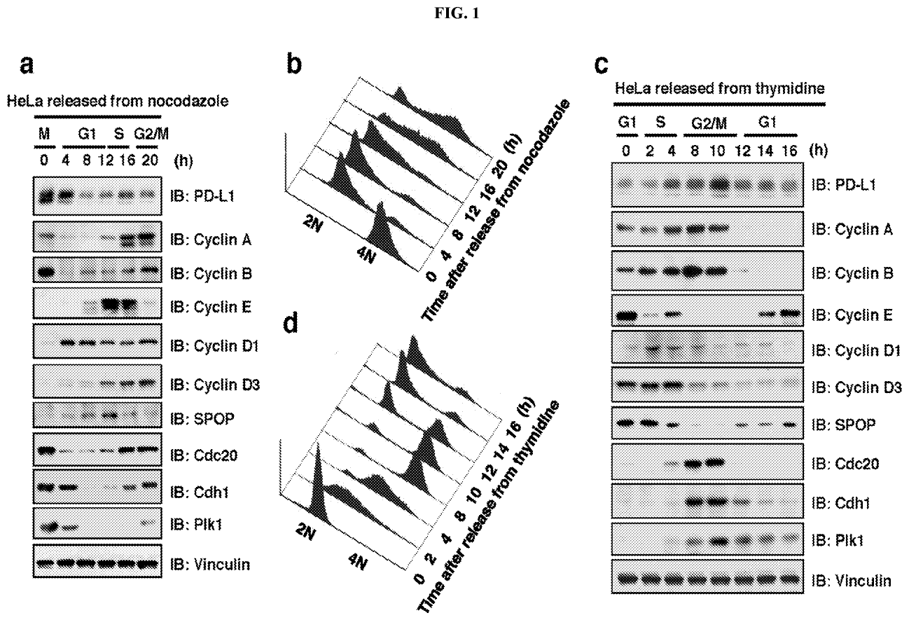

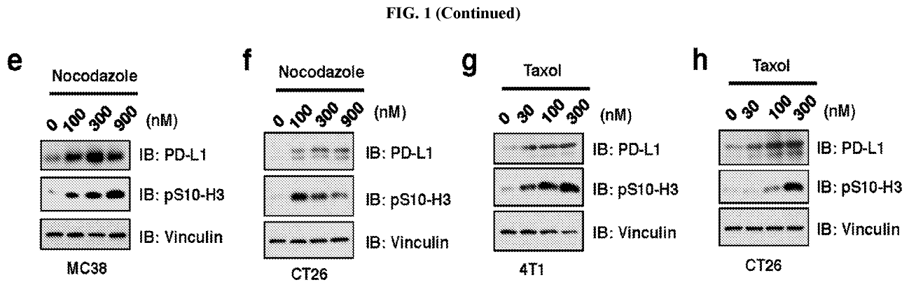

Dysregulated cell cycle progression is one of the hallmarks of human cancers, and targeting cyclin-dependent kinases (CDKs) to block cell cycle transitions has been validated as an effective therapy for cancer patients. Although it has been reported that PD-L1 expression can be regulated at both transcriptional and post-translational levels, it remains largely unknown whether PD-L1 stability is regulated under physiological conditions such as during cell cycle progression. To address this question, multiple cell lines, including HeLa, MDA-MB-231 and HCC1954, were synchronized at M phase using nocodazole treatment, and then released back into the cell cycle (FIG. 1A, FIG. 1B, FIG. 5C, and FIG. 5D). Notably, the protein abundance of PD-L1, similar to mitotic cyclins, fluctuated during cell cycle and displayed a peak at M and early G1 phase, followed by a sharp reduction in late G1 and S phase (FIG. 1A, FIG. 1B). However, there was no significant change of PD-L1 mRNA levels detected during the cell cycle, suggesting that the fluctuation of PD-L1 protein levels during the cell cycle might be regulated at the post-translational level (FIG. 5A, FIG. 5B). Moreover, the fluctuation of PD-L1 protein levels was also observed in HeLa cells synchronized by double thymidine blockage to arrest cells at late G1 phase and released back into the cell cycle (FIG. 1C, FIG. 1D), or under stimulation conditions such, as IFNγ treatment or c-Myc overexpression (FIG. 5E, FIG. 5F, FIG. 5G, FIG. 5H, and FIG. 5I). Elevated PD-L1 protein abundance was also observed in multiple mouse tumor-derived cell lines including MC38, CT26, 4T1 and B16-F10 that were arrested in M phase by nocodazole or the anti-cancer chemotherapy drug, Taxol (FIG. 1E, FIG. 1F, FIG. 1G, FIG. 1H, FIG. 5H, and FIG. 5I). These results suggested that PD-L1, like mitotic cyclins or Plk1, fluctuated during the cell cycle with its protein abundance peaking during the M phase. However, the molecular mechanism responsible for regulating PD-L1 stability during cell cycle progression currently remains undefined.

### Example 2: Inhibition of Cyclin D/CDK4 Kinase Activity Elevates PD-L1 Protein Levels

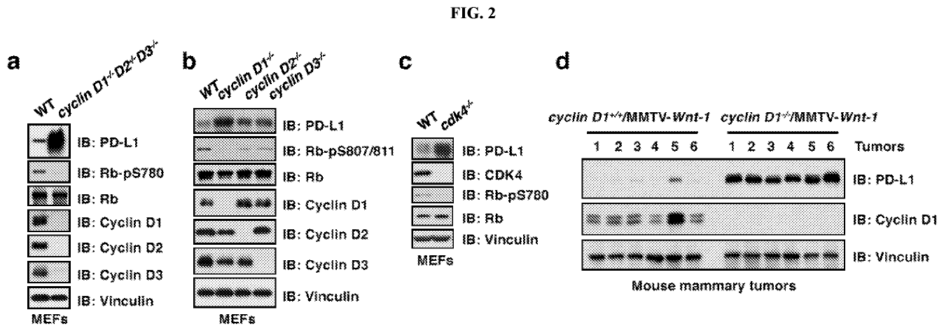

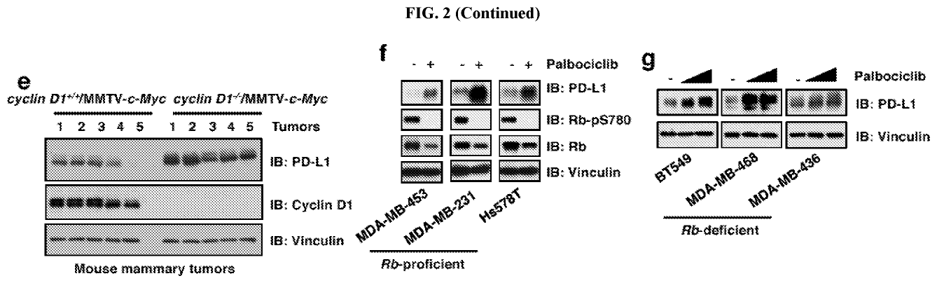

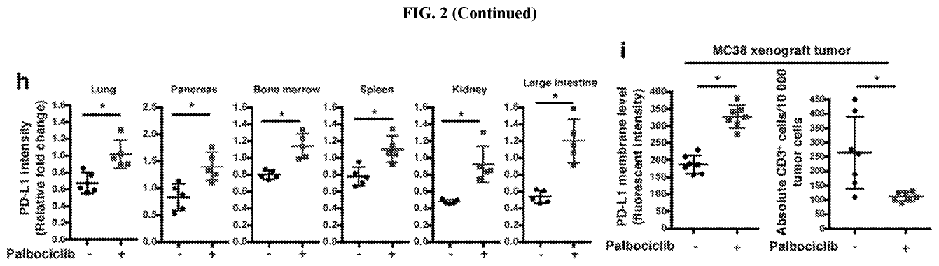

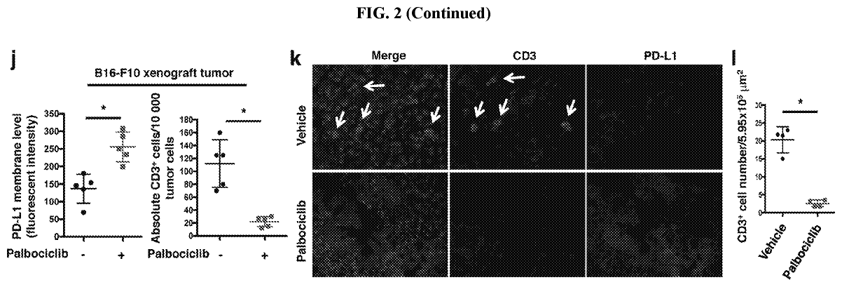

Cyclins and cyclin-dependent kinases play crucial roles in regulating the stability of cell cycle-related proteins during cell cycle progression. Thus, a genetic method was adopted to deplete each major cyclin to explore their potential involvement in regulating the protein stability of PD-L1. Notably, it was found that depleting all three cyclin D isoforms (D1, D2 and D3), dramatically elevated PD-L1 protein abundance in mouse embryonic fibroblasts (MEFs), whereas neither cyclin A (A1 and A2) nor cyclin E (E1 and E2) depletion had this effect (FIG. 2A, FIG. 6A, and FIG. 6B). Moreover, compared to wild-type (WT) MEFs, neither mRNA level of PD-L1 nor cell cycle profile changed dramatically in cyclin D1^{-/-}D2^{-/-}D3^{-/-}MEFs (FIG. 6C, FIG. 6D), suggesting that the elevated protein abundance of PD-L1 might be largely regulated at the post-translational level. Using MEFs genetically depleted of each isoform of cyclin D, it was found that depletion of cyclin D1, and to a lesser extent cyclin D2 or cyclin D3, upregulated PD-L1 protein levels (FIG. 2B). In keeping with this finding, acutely depleting either both cyclin D1 and cyclin D3 in cyclin D1^{fl/fl}D2^{-/-}D3^{fl/fl} MEFs or each of them in breast cancer cell line MDA-MB-231 dramatically elevated PD-L1 protein abundance (FIG. 6E, FIG. 6F). Conversely, reintroducing cyclin D1, and to a lesser extent, cyclin D2 or cyclin D3, could suppress PD-L1 protein abundance in cyclin D1^{-/-}D2^{-/-}D3^{-/-}MEFs (FIG. 6G). Taken together, these results demonstrate that cyclin D1 might be the major cyclin responsible for negatively regulating PD-L1 protein abundance during cell cycle progression.

Consistent with the findings that cyclin D, but not cyclin A or cyclin E, suppressed the PD-L1 protein abundance in cells, depletion of the cyclin D-binding partner CDK4 increased PD-L1 protein abundance whereas depletion of CDK2, a binding partner of cyclin A and cyclin E, did not (FIG. 2C, FIG. 6H, and FIG. 6I). Although cyclin D also binds with CDK6 to form a functional kinase complex, depletion of CDK6 did not affect the PD-L1 protein level (FIG. 6J and FIG. 6K). In further support of a physiological role for cyclin D1 in negatively regulating PD-L1 protein level in vivo, it was found that in mouse breast tumor samples induced by MMTV-Wnt1 or MMTV-c-Myc, PD-L1 expression was dramatically elevated in Cyclin D1^{-/-} genetic background compared to a WT background (FIG. 2D and FIG. 2E). Furthermore, ectopic expression CDK4 WT, but not the kinase-dead N158F mutant, decreased PD-L1 levels in cells (FIG. 6L, FIG. 6M). Conversely, selective CDK4/6 inhibitors, palbociclib or ribociclib, both of which have been used clinically to treat breast cancer patients, unregulated PD-L1 protein abundance in multiple cancer cell lines (FIG. 2F, FIG. 2G, FIG. 6N, FIG. 6O, and FIG. 6P). Moreover, palbociclib treatment could markedly prolong the half-life of PD-L1 (FIG. 6Q and FIG. 6R). As palbociclib and ribociclib displayed similar efficacy in blocking CDK4/Cyclin D-dependent phosphorylation of Rb and elevating PD-L1 protein abundance (FIG. 6O and FIG. 6P), for simplicity, the remainder of the study focused on using palbociclib to understand its mechanism of elevating PD-L1.

The retinoblastoma protein (Rb) is a major target of CDK4/6, whose tumor suppressive function is frequently compromised in human cancer. In keeping with previous studies, a panel of 19 human cancer cell lines was examined and it was found that Rb loss typically led to elevation of CDK4 endogenous inhibitor, p16, which further positively correlated with increased PD-L1 expression levels (FIG. 6S). On the other hand, in Rb-proficient/p16-low cancer cell lines, higher PD-L1 expression correlated with relatively low CDK4 expression (FIG. 6S), further documenting an intrinsic correlation between low CDK4 activity and elevation in PD-L1 expression. Consistently, ectopic expression of p16 in Rb-proficient/p16-low cell lines (MCF7 and T47D) or Rb-deficient/p16-low cell line (HLF) elevated PD-L1 protein abundance (FIG. 6T, FIG. 6U, and FIG. 6V), while depleting p16 in Rb-deficient/p16-high cell lines (MDA-MB-436, BT549, and HCC1937) decreased PD-L1 levels (FIG. 6W, FIG. 6X, and FIG. 6Y). Together, these results support an important role for the Rb/p16/CDK4 axis in regulating PD-L1 protein abundance.

Physiologically, it was found that palbociclib treatment significantly elevated PD-L1 protein levels in all of the 14 different mouse tissues that were examined, including lung, heart, pancreas, bone marrow, spleen, kidney, stomach, large intestine, brain, cerebellum, liver, mammary gland, uterus and ovary (FIG. 2H, FIG. 7A, and FIG. 7B). Furthermore, it was found that PD-L1 expression varies dramatically among different tissues with high expression in spleen, thymus and mammary gland, but relative low expression in remaining tissues, including brain, cerebellum, pancreas, kidney, large intestine and stomach (FIG. 7C and FIG. 7D). However, immune-purified CDK4/cyclin D1 complex from not actively dividing tissues, such as liver and brain, still displayed functional kinase activity towards phosphorylating Rb in vitro (FIG. 7E), which might explain why CDK4/6 inhibitor can elevate PD-L1 protein abundance in tissues without active cell division events.

As a functional consequence, it was found that palbociclib-induced upregulation of PD-L1 was coupled with a concurrent decrease in absolute number of CD3+ TILs in tumors derived from transplanted MC38 and B16-F10 mouse cancer cell lines, as well as in autochthonous mice mammary tumors induced by MMTV-ErbB2 (FIG. 2I, FIG. 2J, FIG. 2K, FIG. 2L, FIG. 7F, and FIG. 7G). These results together demonstrated that inhibition of cyclin D/CDK4 kinase activity by palbociclib elevated PD-L1 protein levels in both in vitro cell culture experimental systems and in vivo mouse models, which might contribute to the observed resistance to CDK4/6 inhibitor treatment during cancer therapy. However, the molecular mechanism of how cyclin D/CDK4 governs PD-L1 protein stability remains largely unexplored.

### Example 3: Cancer-Derived SPOP Mutants Promote Tumor Growth Largely Through Increased Immune Evasion Mediated by their Deficiency in Promoting PD-L1 Degradation

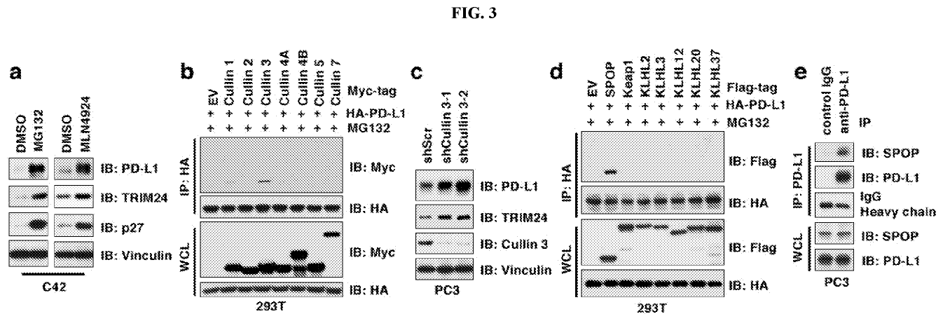

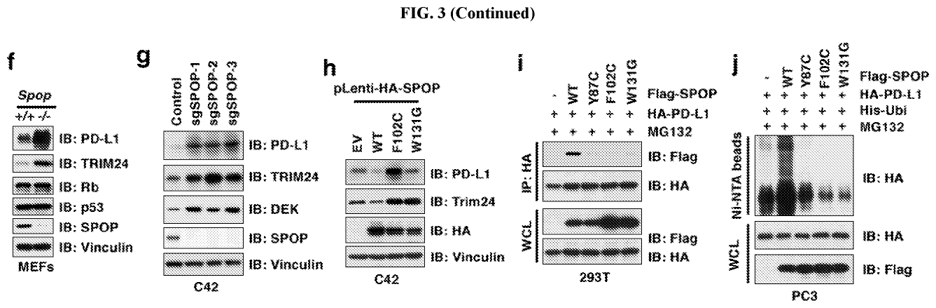

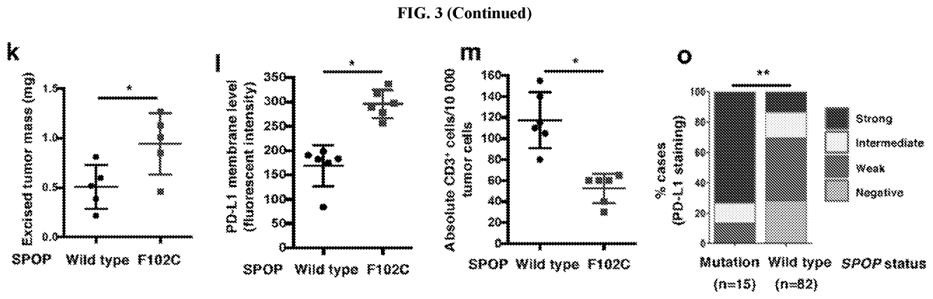

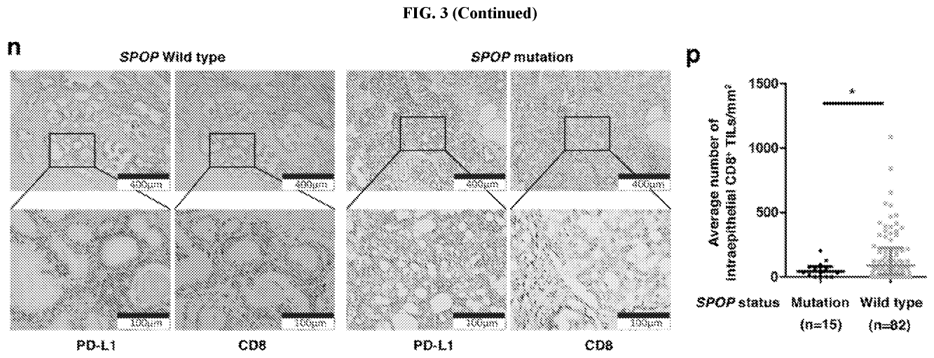

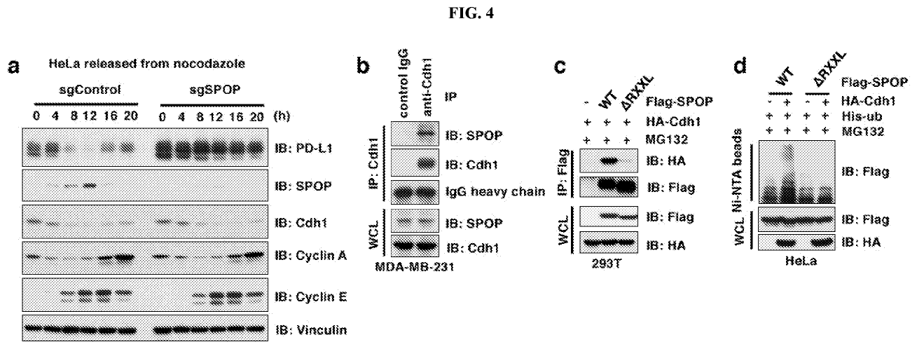

The ubiquitin-proteasome system (UPS) is the most important pathway for regulating protein stability and is responsible for controlling multiple cellular processes including cell cycle progression. To further explore whether the UPS is involved in suppressing PD-L1 stability, cells were treated with the proteasome inhibitor, MG132, and cullin-based ubiquitin E3 ligase inhibitor, MLN4924, and it was found that both inhibitors stabilized PD-L1 protein in cells (FIG. 3A). This result suggested that Cullin-Ring ligases (CRLs) might be the physiological upstream E3 ligase(s) governing PD-L1 stability. Through screening for interaction of PD-L1 with cullin family proteins, it was found that Cullin 3, and to a lesser extent, Cullin 1, but none of the other cullin family members examined, interacted with PD-L1 in cells (FIG. 3B). However, the cytoplasmic carboxyl-terminal amino acid tail (260-290) of PD-L1 (termed PD-L1 C-tail hereafter) specifically interacted only with Cullin 3, but not Cullin 1 or other cullin family members in cells (FIG. 8A and FIG. 8B), which indicates that in addition to the reported Cullin 1/β-TRCP¹¹, Cullin 3-based E3 ligase(s) might play an important role in regulating PD-L1 stability. In keeping with this notion, depletion of Cullin 3 markedly elevated the protein abundance of endogenous PD-L1 in cells (FIG. 3C).

Cullin 3-based E3 ubiquitin ligases recognize their downstream substrates through one of several adaptor proteins, which typically contain one BTB domain to interact with Cullin 3 and one substrate-recognizing motif to recruit the specific substrate. It was found that SPOP, but not any of the other examined adaptor proteins, including Keap1, KLHL2, KLHL3, KLHL12, KLHL20, or KLHL37, specifically interacted with PD-L1 in cells (FIG. 3D and FIG. 3E). Furthermore, SPOP specifically interacted with the C-tail of PD-L1 and, deleting the C-tail of PD-L1 (A760-290) disrupted binding with SPOP and PD-L1 became resistant to SPOP-meditated poly-ubiquitination (FIG. 8C, FIG. 8D, and FIG. 8E). However, deletion of the PD-L1 C-tail did not affect its interaction with β-TRCP, which interacted with PD-L1 largely through the β-TRCP-binding motif locating in the extracellular domain of PD-L1¹¹ (FIG. 8F). This result indicates that under physiological conditions, the functional PD-L1 species inserted in the plasma membrane might be targeted for degradation by Cullin 3^{SPOP}, whereas Cullin1^{β-TRCP} might govern PD-L1 degradation before its insertion into the plasma membrane to become fully functional.

The last eight amino acids of PD-L1 (283-290) were further identified as the potential binding motif (also called degron) for SPOP, as the PD-L1 (Δ283-290) mutant failed to bind with SPOP and became resistant to SPOP-mediated degradation (FIG. 8G and FIG. 8H). Importantly, the cancer-derived PD-L1 T290M mutant (cBioPortal, sample ID: TCGA-IB-7651-01 and TCGA-BR-4362-01) located in the SPOP-binding motif lost its ability to interact with SPOP and became more stable largely through decreasing SPOP-mediated poly-ubiquitination and degradation (FIG. 8G, FIG. 8H, FIG. 8I, FIG. 8J, FIG. 8K, and FIG. 8L). Although the frequency of PD-L1 mutation is not high in human cancer, mutations in the PD-L1 C-tail (degron) are mutually exclusive with mutations in the substrate-interacting MATH domain of SPOP (FIG. 8M). These results demonstrated that SPOP interacts with and promotes PD-L1 degradation largely through the last C-terminal eight amino acids in PD-L1, which resemble a phospho-derivative of the canonical SPOP degron motif present in SRC3 or ERG (FIG. 8A).

In keeping with the result that SPOP specifically interacts with PD-L1 in cells, ectopic expression of SPOP, but not Keap1 or hCop1, markedly reduced PD-L1 protein abundance in cells (FIG. 9A, FIG. 9B, and FIG. 9C). Moreover, deleting the MATH domain of SPOP disrupted its interaction with PD-L1 and abrogated its ability to promote PD-L1 degradation in cells (FIG. 9D, FIG. 9E, and FIG. 9F). Consistent with the notion that SPOP negatively regulated PD-L1 protein stability, ectopic expression of SPOP shortened the half-life of PD-L1 and promoted the poly-ubiquitination of PD-L1 in cells (FIG. 9G, FIG. 9H, and FIG. 9I). Conversely, the PD-L1 protein abundance was dramatically elevated in Spop^{-/-}MEFs compared to Spop^{+/+}MEFs, but no significant change of PD-L1 mRNA levels was observed between Spop^{+/+} and Spop^{-/-}MEFs (FIG. 3F and FIG. 9J). Moreover, depletion of SPOP using CRISPR-Cas9 technology or shRNAs also led to a marked elevation of PD-L1 protein levels in cells (FIG. 3G and FIG. 9K), and significantly prolonged the half-life of endogenous PD-L1 protein (FIG. 9L and FIG. 9M). Notably, depleting known SPOP substrates including AR, ERG, Trim24 or DEK in cells with WT or SPOP^{-/-} genetic backgrounds did not lead to obvious changes of PD-L1 (FIG. 9N, FIG. 9O, FIG. 9P, FIG. 9Q, FIG. 9R, FIG. 9S, FIG. 9T, and FIG. 9U), excluding the possibility of secondary effects for the observed elevation of PD-L1 upon depleting SPOP.

Although it has been reported that SPOP mutations occur in approximately 10-15% of human prostate cancers (PrCa), SPOP mutations were analyzed in all cancer types from the TCGA database and it was found that recurrent hotspot mutations in SPOP largely occur in prostate adenocarcinoma (PRAD, 11%), uterine corpus endometrial carcinoma (UCEC, 14%), and uterine carcinosarcoma (UCS, 7%) (FIG. 10A). Moreover, most of the identified SPOP somatic mutations in PrCa including Y87C, F102C and W131G, are clustered in the N-terminal substrate-interacting MATH domain (FIG. 10B), which makes them deficient in interacting and promoting substrate poly-ubiquitination and degradation. Indeed, compared with wild-type SPOP (SPOP-WT), all cancer-derived SPOP mutations examined, including Y87C, F102C and W131G, failed to promote PD-L1 degradation largely due to their deficiency in binding with PD-L1 and earmarking PD-L1 for poly-ubiquitination (FIG. 3H, FIG. 3I, FIG. 3J, and FIG. 10C). To further examine whether cancer patient-derived SPOP mutations affect the PD-L1 level and TILs in transplanted tumors, B16-F10 stable cell lines ectopically expressing SPOP-WT were generated, as well as cancer-derived Y87C, F102C, and W131G mutants, and an empty vector (EV) was used as a negative control. It was found that ectopic expression of SPOP-WT, but not SPOP mutants, including Y87C, F102C or W131G in B16-F10 mouse cancer cells decreased the abundance of endogenous PD-L1 (FIG. 10D). Consistently, the growth of implanted tumors expressing cancer-derived mutant SPOP-F102C was faster than tumors expressing SPOP-WT in immunocompetent C57BL/6 mice, as evaluated by the weight of excised tumors (FIG. 3K and FIG. 10E). Moreover, compared with tumors expressing SPOP-WT, upregulation of PD-L1 coupled with decreased CD3⁺ TILs was observed in both B16-F10 and 4T1 tumors expressing SPOP-F102C (FIG. 3L, FIG. 3M, FIG. 10F, and FIG. 10G). However, there was no significant difference between B16-F10 or 22RV1 SPOP-WT and SPOP-F102C in in vitro cell growth or implanted tumor growth in T cell-deficient TCRα KO mice (FIG. 10H, FIG. 10I, FIG. 10J, FIG. 10K, FIG. 10L, and FIG. 10M). Furthermore, compared to control IgG treatment, anti-PD-L1 antibody treatment largely alleviated the difference of tumor weights between SPOP-WT and SPOP-F102C groups implanted in immune-competent C57BL/6 mice (FIG. 10N, FIG. 10O, FIG. 10P, and FIG. 10Q). These results demonstrated that cancer-derived SPOP mutants promote tumor growth largely through increased immune evasion mediated by their deficiency in promoting PD-L1 degradation.

### Example 4: Cdh1 is a Physiologically Important Upstream E3 Ligase Responsible for Negatively Regulating SPOP Protein Stability During Cell Cycle Progression

Furthermore, it was explored whether loss-of-function SPOP mutations in PrCa correlated with elevated PD-L1 and decreased TILs in human PrCa. To this end, anti-PD-L1 and anti-CD8 antibodies were first validated through several methods, including immunoblot, immunofluorescence, and immunohistochemistry (IHC) (FIG. 11A, FIG. 11B, FIG. 11C, FIG. 11D, FIG. 11E, FIG. 11F, FIG. 11G and FIG. 11H). Subsequently, PD-L1 protein abundance and CD8⁺ cell population were analyzed in 97 human primary prostate tumor specimens. 15 SPOP-mutation and 82 SPOP-wild type cases were identified through large-scale sequencing as described previously. IHC staining of PD-L1 and CD8 were performed in these prostate tumor specimens (FIG. 3N). Notably, IHC results showed that approximately 80% of SPOP-mutated tumors exhibited strong PD-L1 staining (FIG. 3N and FIG. 3O). However, only approximately 10% of SPOP-WT tumors exhibited strong staining of PD-L1 protein and 70% of SPOP-WT cases exhibited weak or no staining of PD-L1 (FIG. 3N and FIG. 3O). Moreover, compared with SPOP-WT samples, CD8⁺ TILs were dramatically decreased in the samples harboring SPOP mutations (FIG. 3P). These results support the model that SPOP-deficiency positively correlates with an elevation in PD-L1 protein abundance and decreased TILs in the PrCa clinical setting.

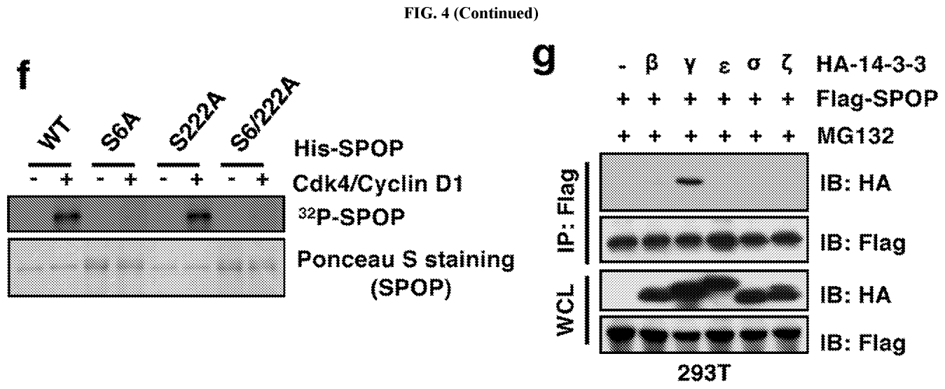

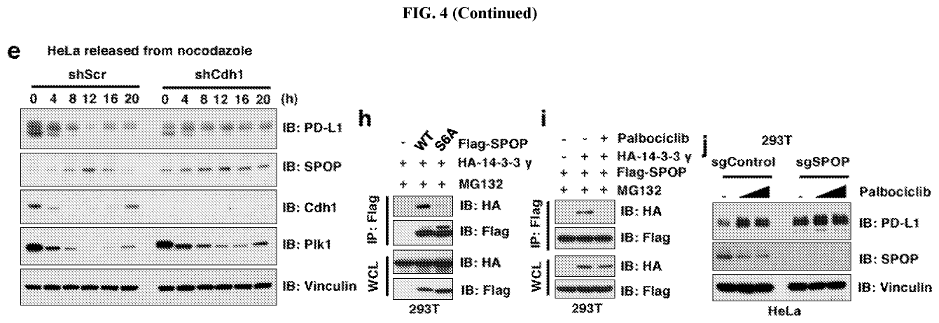

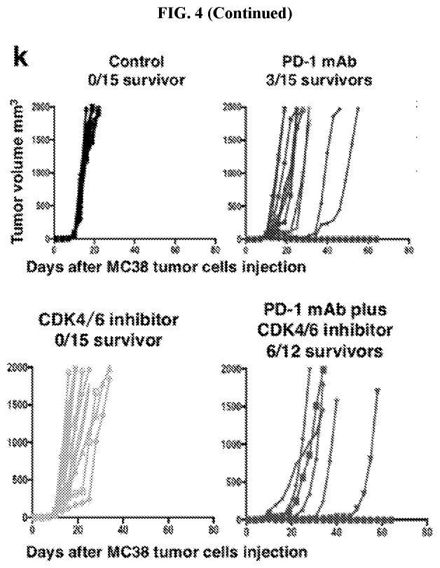

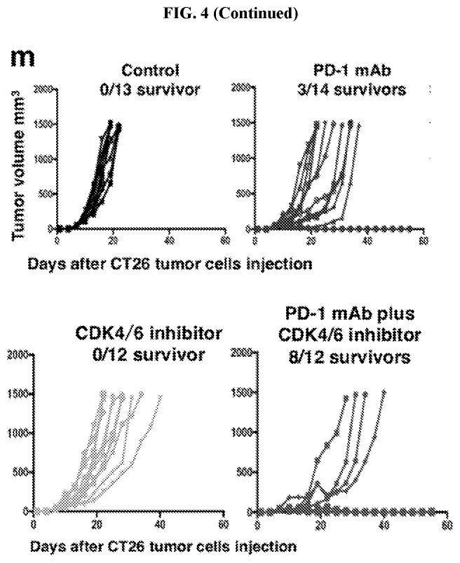

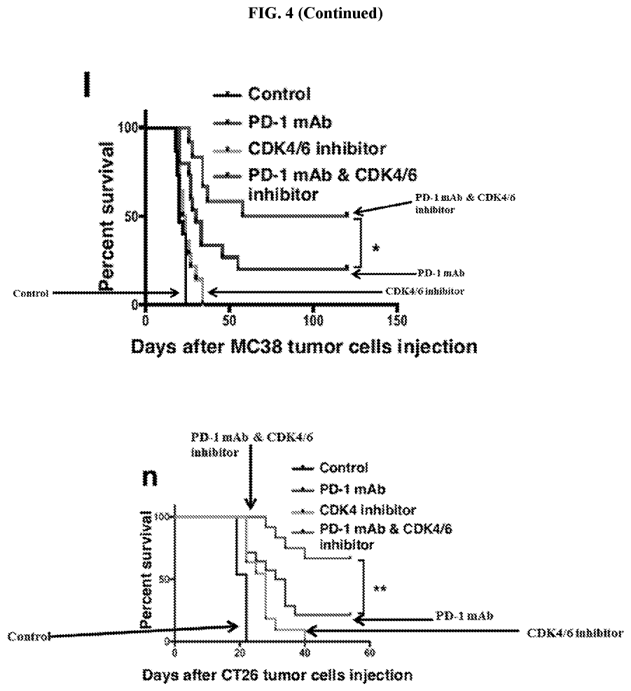

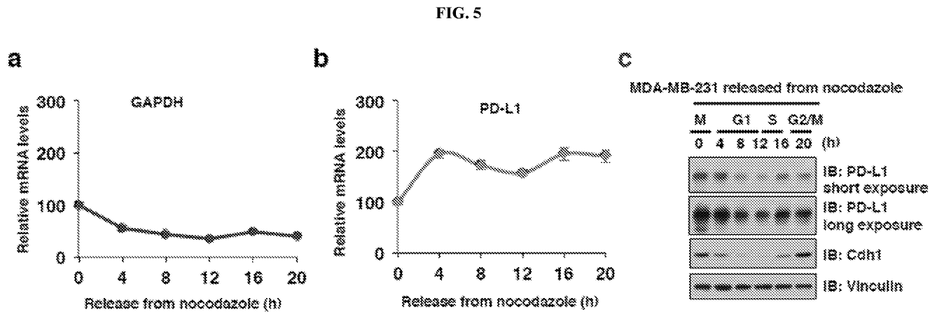

To further explore the physiological function of SPOP in regulating PD-L1 stability during cell cycle progression, it was confirmed that SPOP protein abundance also fluctuated during the cell cycle and displayed an inverse correlation with PD-L1 protein levels (FIG. 1A, FIG. 1C, and FIG. 4A). More importantly, it was demonstrated that depletion of endogenous SPOP using CRISPR technology resulted in stabilization of PD-L1 across the cell cycle (FIG. 4A and FIG. 12A), which suggests that the PD-L1 stability was negatively regulated by SPOP in a cell cycle-dependent manner. However, it remains unknown how SPOP stability is regulated during cell cycle progression and its relationship to CDK4/Cyclin D kinase complex. To this end, it was observed that the Anaphase-Promoting Complex/Cyclosome (APC/C) E3 ligase adaptor protein Cdh1 displayed an inverse correlation with SPOP protein levels during cell cycle (FIG. 1A, FIG. 1C, and FIG. 4A). Given the critical role of APC/C E3 ligase complex in cell cycle progression, especially in M and early G1 phases, these results indicated that the APC/C^{Cdc20} or APC/C^{Cdh1} might be the upstream E3 ligase to negatively regulate PD-L1 stability. In keeping with this notion, it was found that both Cdc20 and Cdh1 could interact with SPOP in cells, although Cdh1 had a higher binding affinity with SPOP than Cdc20 (FIG. 12B). Depletion of Cdh1, but not Cdc20, elevated SPOP protein abundance, which was coupled with a simultaneous reduction in PD-L1 protein abundance (FIG. 12C and FIG. 12D). These results supported the notion that APC/C^{Cdh1} might be the specific physiological E3 ligase responsible for negatively regulating SPOP stability during cell cycle progression.

In keeping with this hypothesis, the interaction between SPOP and Cdh1 was detected at the endogenous level in cells (FIG. 4B and FIG. 12E). Importantly, it was found that SPOP contained an evolutionarily conserved destruction box motif (D-box: RxxLxxxxN) that played a crucial role in mediating Cdh1-dependent poly-ubiquitination and subsequent degradation of its downstream targets (FIG. 12F). Consistently, ablation of the D-box through deletion of RxxL in SPOP disrupted its binding with Cdh1 and rendered SPOP resistant to Cdh1-mediated poly-ubiquitination and degradation (FIG. 4C, FIG. 4D, and FIG. 12G), which dramatically prolonged the half-life of SPOP (FIG. 12H and FIG. 12I). In further support of a physiological role of Cdh1 in dictating periodic expression of SPOP during the cell cycle, depletion of Cdh1 led to stabilization of SPOP across the cell cycle and was coupled with a reduction in PD-L1 protein level during cell cycle progression (FIG. 4E). Taken together, these results demonstrate that Cdh1 is a physiologically important upstream E3 ligase responsible for negatively regulating SPOP protein stability during cell cycle progression.

To further gain insight into how cyclin D/CDK4 regulates PD-L1 during cell cycle progression through disrupting the Cdh1/SPOP axis, it was found that cyclin D1/CDK4 directly phosphorylates SPOP at Ser6, but not Ser222, the only two conserved SP sites in SPOP (FIG. 4F and FIG. 13A). In keeping with the result that SPOP is a cyclin D1/CDK4 substrate, a strong interaction between SPOP and CDK4, but not CDK2 or CDK6, was observed in cells (FIG. 13B). In keeping with the notion that all three cyclin D isoforms negatively regulated PD-L1 stability (FIG. 2B and FIG. 6G), it was found that all three D-type cyclin/CDK4 complexes could phosphorylate SPOP in vitro (FIG. 13C and FIG. 13D). Furthermore, Ser6 phosphorylation status in SPOP-WT, but not the S6A mutant, could be recognized by the phospho-Ser-Pro-Pro motif antibody in cells, which could be suppressed by the CDK4/6 inhibitor, palbociclib (FIG. 13E).

Interestingly, it was noticed that one canonical 14-3-3 protein binding motif (RxxpS/pTxP) was located at Ser6 in SPOP (FIG. 13A). Indeed, 14-3-3γ, but not other isoforms, specifically interacted with SPOP in cells (FIG. 4G), which was likely due to a direct interaction between 14-3-3γ and pSer6 species of SPOP (FIG. 13F). As such, the phosphorylation-deficient SPOP-S6A mutant lost its ability to interact with 14-3-3γ (FIG. 4H). Moreover, ectopic expression of CDK4 elevated the interaction of SPOP-WT, but not the S6A mutant, with 14-3-3γ (FIG. 13G). Furthermore, it was found that 14-3-3γ could disrupt SPOP interaction with Cdh1 in a dose-dependent manner, which suggested that 14-3-3γ might stabilize SPOP through binding with cyclin D/CDK4-mediated phosphorylation of SPOP (FIG. 13H).

Although the phosphorylation-deficient S6A mutant of SPOP did not affect its ability to interact with Cullin 3 and self-dimerize (FIG. 13I and FIG. 13J), the SPOP-S6A mutant displayed an enhanced interaction with its upstream E3 ligase adaptor protein, Cdh1 (FIG. 13K). Consistently, compared to SPOP-WT, the SPOP-S6A mutant exhibited a shorter half-life and increased poly-ubiquitination (FIG. 13L, FIG. 13M, and FIG. 13N). As such, the CDK4/6 inhibitor, palbociclib, could decrease the interaction of SPOP with 14-3-3γ and subsequently enhance its binding with Cdh1, leading to elevated SPOP poly-ubiquitination in cells (FIG. 4I, FIG. 13O, and FIG. 13P). In support of these findings, it was found that palbociclib treatment dramatically elevated PD-L1 levels accompanied with decreasing SPOP protein abundance in SPOP-WT, but not SPOP-deficient cells (FIG. 4J). In keeping with 14-3-3γ stabilizing SPOP to promote PD-L1 degradation, depletion of 14-3-3γ dramatically upregulated PD-L1 levels and stabilized PD-L1 during cell cycle progression (FIG. 13R, FIG. 13S, FIG. 13T, and FIG. 13U).

### Example 5: Combination Treatment with Anti-PD-1 Antibody and a CDK4/6 Inhibitor was Highly Effective in Reducing Tumor Growth

Recent clinical studies revealed that resistance to CDK4/6 inhibitor treatment often develops during cancer therapy. However, the molecular mechanism(s) for CDK4/6 inhibitor resistance remains largely unknown. The results herein demonstrate that CDK4/6 inhibitor treatment could elevate PD-L1 protein levels, allowing tumor cells to evade cancer immune-surveillance. Given this scenario, it was hypothesized that the combination of CDK4/6 inhibitor and anti-PD-1/PD-L1 antibody treatment may overcome CDK4/6 inhibitor-mediated cancer immune evasion by eliciting immune-mediated anti-tumor immunity. To examine this hypothesis, the combination treatment of MC38 subcutaneous tumor mouse model with anti-PD-1 (1A12) antibody and the CDK4/6 inhibitor palbociclib was assessed (see treatment regimen in FIG. 14A). It was found that the CDK4/6 inhibitor (palbociclib) slightly retarded tumor growth and extended survival of mice when compared to the control group. Anti-PD-1 (1A12) treatment retarded tumor progression and resulted in 3 complete responses out of fifteen treated mice. Strikingly, the combination treatment of palbociclib treatment and anti-PD-1 antibody dramatically retarded tumor progression and resulted in 6 complete responses out of 12 treated mice (FIG. 4K). Moreover, the combination of CDK4/6 inhibitor with anti-PD-1 therapy resulted in a significant improvement in overall survival compared with control group or single agent treated group (FIG. 4L). Furthermore, changes of absolute CD3⁺ TIL as well as Granzyme B⁺ and IFNγ⁺ of CD3⁺ cells were also compared after a single agent treatment or combinational treatment. Notably, single agent CDK4/6 inhibitor palbociclib treatment significantly reduced the absolutely number of CD3⁺ and CD8⁺ TILs, as well as the frequency of CD8⁺ cells in CD3⁺ cell population, which could be rescued by combination of CDK4/6 inhibitor palbociclib plus anti-PD1 treatment (FIG. 14B, FIG. 14C, and FIG. 14D). Moreover, the absolute number or Granzyme B⁺ and IFNγ⁺ of CD3⁺ cells were also reduced after CDK4/6 inhibitor treatment, which could be further recovered by a combination of CDK4/6 inhibitor palbociclib plus anti-PD1 treatment (FIG. 14E, FIG. 14F, FIG. 14G, and FIG. 14H). More importantly, similar to MC38 implanted tumors, CDK4/6 inhibitor combination with anti-PD-1 antibody of CT-26 implanted can dramatically inhibit tumor growth rate and enhance survival rate of mice (FIG. 4M and FIG. 4N). Hence, the results showed that combination treatment with anti-PD-1 antibody and a CDK4/6 inhibitor was highly effective in reducing tumor growth and enhancing the overall survival of mice in an immune-competent mouse model.

Given that the PD-1/PD-L1 pathway plays a crucial role in tumor immune evasion, but only about 20-30% of tumors respond to PD-1/PD-L1 antibody treatment, it is promising to explore combinations of PD-1/PD-L1 immune checkpoint blockade with targeted and conventional cancer therapies to enhance response rates and benefit more cancer patients. The examples of this disclosure showed that PD-L1 protein abundance is negatively regulated during cell cycle progression by cyclin D/CDK4 and the Cullin 3^{SPOP} E3 ligase in a proteasome-mediated degradation manner. Importantly, CDK4/6 inhibitors treatment could elevate PD-L1 protein levels largely through inhibiting cyclin D/CDK4-mediated phosphorylation of SPOP to promote its degradation by APC/C^{Cdh1} (FIG. 14I). Thus, without wishing to be bound to theory, upregulation of PD-L1 by CDK4/6 inhibition might help tumors evade immune surveillance, which could be one possible reason for underlying acquired drug resistance for CDK4/6 inhibitors in clinical therapy. The results of this example demonstrated that combining CDK4/6 inhibitor treatment with anti-PD-1 immunotherapy enhanced tumor regression and dramatically improved overall survival rates. Taken together, the present invention uncovered a novel molecular mechanism for regulating

PD-L1 stability during cell cycle progression and shows the potential for combination treatment with CDK4/6 inhibitors and PD-1/PD-L1 immune checkpoint blockade to enhance therapeutic efficacy for human cancers.

The results described herein above, were obtained using the following methods and materials.

### Cell Culture, Transfections and Viral Infections

HEK293T, HEK293, HeLa, MDA-MB-231, MCF7, Hs578T, WT MEFs, cyclin D1^{-/-} MEFs, cyclin D2^{-/-}MEFs, cyclin D3^{-/-} MEFs, cyclin D1^{-/-}D2^{-/-}D3^{-/-}MEFs, cyclin D1^{fl/fl}D2^{-/-}D3^{fl/fl}, Cdk4^{-/-} and Cdk4^{-/-} MEFs, cyclin A1^{+/+}A2^{+/+} and cyclin A1^{-/-}A2^{-/-} MEFs, cyclin E1^{+/+}E2^{+/+} and cyclin E1^{-/-}E2^{-/-}, Spop^{+/+} and Spop^{-/-}MEFs (a kind gift of Dr. Nicholas Mitsiadesa, Baylor College of Medicine, Houston, Tex.) were cultured in DMEM medium supplemented with 10% FBS (Gibco), 100 units of penicillin and 100 μg/ml streptomycin (Gibco). HLF, HepG2, Huh1 and Huh7 were cultured in RPMI medium supplemented with 10% FBS. MDA-MB-231 PD-L1 WT and PD-L1 KO cells are kind gift from Dr. Mien-Chie Hung. BT549, T47D, ZR75-1, HCC1954, HCC1937, MDA-MB436, MDA-MB468 and SKBR3 cells were from Dr. Alex Toker laboratory at BIDMC, Harvard

Medical School, and cultured in RPMI medium or McCoy′s5A (Corning, N.Y.) medium supplemented with 10% FBS. PC3, DU145, 22RV1, LNCaP and C42 were kind gifts from Dr. Pier Paolo Pandolfi group at BIDMC, Harvard Medical School, and cultured in RPMI medium (Corning, N.Y.) with 10% FBS. Mouse tumor derived MC38 cell line was a kind gift from Dr. Arlene Sharpe at Harvard Medical School. Mouse tumor derived 4T1 and B16-F10 cell lines were routinely cultured in Gordon Freeman's laboratory in DMEM medium supplemented with 10% FBS (Gibco), 100 units of penicillin and 100 μg/ml streptomycin (Gibco). All cell lines were routinely tested to be negative for mycoplasma contamination.

Cells with 80% confluence were transfected using lipofectamine plus reagents in Opti-MEM medium (Invitrogen). 293FT cells were used for packaging of lentiviral and retroviral cDNA expressing viruses, as well as subsequent infection of various cell lines were performed according to the protocols described previously³⁷. Briefly, medium with secreted viruses were collected twice at 48 hours and 72 hours after transfection. After filtering through 0.45 μM filters, viruses were used to infect cells in the presence of 4 μg/mL polybrene (Sigma-Aldrich). 48 hours post-infection, cells were split and selected using hygromycin B (200 μg/mL) or puromycin (1 μg/mL) for 3 days. Cells were harvested and lysed in EBC buffer (50 mM Tris pH 7.5, 120 mM NaCl, 0.5% NP40) supplemented with protease inhibitors (Roche) and phosphatase inhibitors (Calbiochem) for immunoblot analysis.

### Reagents

Nocodazole (M1404) and Taxol were purchased from Sigma. Thymidine (CAS: 50-89-5) and cycloheximide (66-81-9) were purchased from Acros organics. PD0332991 (S1116) was purchased from Selleckchem. MG132 (BML-PI102-0005) was purchased from Enzo life science. MLN4924 was a kind gift from Dr. William Kaelin (Dana-Farber cancer institute).

### Plasmids

Myc-tagged Cullin 1, Cullin 2, Cullin 3, Cullin 4A, Cullin 4B, Cullin 5, Flag-tagged SPOP WT, Y87C, F102C, W131G, delta MATH, delta BTB, pLenti-HA-SPOP WT, Y87C,

F102C, W131G, pGEX-4T-1-SPOP, Flag-Keap1, Flag-Cop1, shScramble, shCullin 3, shSPOP, and His-ubiquitin constructs were described previously³⁸. Myc-Cullin 7 construct was kindly offered by Dr. James A. DeCaprio (Dana-Farber Cancer Institute). KLHL2 and KLHL3 constructs were generous gifts from Dr. Shinichi Uchida (Tokyo Medical and Dental University). KLHL12 and KLHL37 constructs were purchased from Addgene. KLHL20 construct was offered by Dr. Ruey-Hwa Chen (Institute of Biological Chemistry, Academia Sinica, Taiwan). The construct of HA-PD-L1 (HA tag in the N-terminus of PD-L1) was kindly provided by Dr. Mien-Chie Hung (The University of Texas MD Anderson Cancer Center). HA-Cdh1, HA-Cdc20, shCdh1 and shCdc20 were described previously³⁹. HA-14-3-3 isoform constructs were described previously (Gao et al. Nat Cell Biol 11, 397-408 (2009)). pcDNA3-PD-L1, pCMV-GST-PD-L1-tail (cytoplasmic amino acids), Flag-SPOP with delta D-Box (RxxL), Flag-SPOP S6A, HA-tagged CDK2, CDK4 and CDK6 were generated in this study.

### Antibodies

PD-L1 (E1L3N) rabbit mAb (13684), anti-pS10-H3 (3377), anti-pS780-Rb (8180), anti-pS807/811-Rb (8516), anti-Rb (9309), anti-cyclin D1 (2978), anti-cyclin D2 (3741), anti-CDK4, anti-CDK6 (3136), anti-cullin 3 (2759), anti-GST (2625), rabbit polyclonal anti-Myc-Tag antibody (2278) and mouse monoclonal anti-Myc-Tag (2276) antibodies were purchased from Cell Signaling Technology. Mouse PD-L1 antibody (MAB90781-100) was purchased from R&D systems. Anti-mPD-L1 for immunoblotting (clone 298B.8E2), anti-mPD-L1 (clone 298B.3G6) for immunohistochemistry, and anti-human PD-L1 for immunoprecipitation (clone 29E.12B1) were generated in the laboratory of Dr. Gordon J. Freeman. Anti-SPOP antibody (16750-1-AP) was purchased from Proteintech. Anti-cyclin A antibody (sc-751), anti-cyclin B antibody (sc-245), anti-cyclin E (SC-247), anti-cyclin D3 (sc-182), anti-Cdh1 antibody (sc56312), anti-Cdc20 antibody (sc-8358), anti-Cdc20 antibody (sc-13162), anti-Plk1 antibody (sc-17783), anti-TRIM24 (TIF1α, SC-271266), and anti-HA antibody (sc-805, Y-11) and anti-GST (sc-459) were obtained from Santa Cruz. Anti-GFP (8371-2) antibody was purchased from Clontech. Anti-Flag (F-2425), anti-Flag (F-3165, clone M2), anti-Vinculin (V9131), anti-Flag agarose beads (A-2220), anti-HA agarose beads (A-2095), peroxidase-conjugated anti-mouse secondary antibody (A-4416) and peroxidase-conjugated anti-rabbit secondary antibody (A-4914) were purchased from Sigma. Anti-HA (MMS-101P) was obtained from BioLegend.

### Immunoblot and Immunoprecipitation Analyses

Cells were lysed in EBC buffer (50 mM Tris pH 7.5, 120 mM NaCl, 0.5% NP-40) supplemented with protease inhibitors (Complete Mini, Roche) and phosphatase inhibitors (phosphatase inhibitor cocktail set I and II, Calbiochem). Protein concentrations were measured by the Beckman Coulter DU-800 spectrophotometer using the Bio-Rad protein assay reagent as described previously (Gao et al. Nat Cell Biol 11, 397-408 (2009)). Equal amounts of protein were resolved by SDS-PAGE and immunoblotted with indicated antibodies. For immunoprecipitations analysis, 1000 pg total cell lysates were incubated with the primary antibody-conjugated beads for 4 hours at 4° C. The recovered immunocomplexes were washed four times with NETN buffer (20 mM Tris, pH 8.0, 100 mM NaCl, 1 mM EDTA and 0.5% NP-40) before being resolved by SDS-PAGE and immunoblotted with indicated antibodies.

### Immunohistochemistry (IHC) and Evaluation

The prostate tumor specimens were obtained from Shanghai Changhai Hospital in China. Usage of these specimens was approved by the Institute Review Board of Shanghai Changhai Hospital. For IHC, the paraformaldehyde fixed paraffin embedded prostate tumor samples were deparaffinized in xylene (3×10 min), rehydrated through a series of graded alcohols (100%, 95%, 85%, and 75%) to water. Samples were then subjected to heat-mediated antigen retrieval at 95° C. for 20 min. For IHC analysis, we used UltraSensitive™ SP (Mouse) IHC Kit (KIT-9701, Fuzhou Maixin Biotech) following the manufacturer's instructions with minor modification. The sections were incubated with 3% H₂O₂ for 15 min at room temperature to block endogenous peroxidase activity. After incubating in normal goat serum for 1 hour to block non-specific binding of IgG, sections were treated with primary antibody (PD-L1, 298B.3G6, 18 μg/ml; CD8a, sc-53212, clone C8/144B, dilution 1:40) at 4° C. overnight. Sections were then incubated for 30 min with biotinylated goat-anti-mouse IgG secondary antibodies (Fuzhou Maixin Biotech), followed by incubation with streptavidin-conjugated HRP (Fuzhou Maixin Biotech). Specific samples were developed with 3′3-diaminobenzidine (DAB-2031,Fuzhou Maixin Biotech). Images were taken using an Olympus microscopic camera and matched software.

The expression level of PD-L1 in prostate cancer tumor samples was determined according to the intensity of the staining as 0, negative; 1, weak expression; 2, moderate expression and 3, strong expression. The numbers of intraepithelial CD8⁺ tumor-infiltrating T lymphocytes (TILs) was counted. Briefly, three independent areas with the most abundant infiltration were selected under a microscopic field at 200× magnification (0.0625 mm²). The number of intraepithelial CD8⁺ TILs was counted manually and calculated as cells per mm². The Mann-Whitney test was used to compare the difference in PD-L1 expression between SPOP mutated and wide type cases. The Student's t test was used to determine p values of the difference in CD8⁺ TILs between SPOP mutated and wide type cases. p<0.05 was considered as significant.

### In Vitro Cyclin D1/CDK4 Kinase Assays

Cyclin D1/CDK4 in vitro kinase assays were performed as described (Phelps et al. Methods In Enzymology 283, 194-205 (1997)). Briefly, bacterially purified His-SPOP WT,

S6A, S222A, and S6A/S222A were incubated with recombinant human Cdk4/Cyclin D1 protein (ab55695) in kinase buffer (50 mM HEPES, pH 7.0, 10 mM MgCl₂, 5 mM MnCl₂, 1 mM DTT). ATP mix with γ-³²P-ATP was added at a 100 μM final concentration. The reaction was initiated by the addition of Cdk4/Cyclin D1 in a volume of 30 μl for 30 min at 30° C. followed by adding 3×SDS-PAGE sample buffer to stop the reaction before resolution by SDS-PAGE and subsequent autoradiography.

### In Vivo Ubiquitination Assays

PC3 or HeLa cells with 80% confluence were transfected with His-ubiquitin and the indicated constructs. 36 hours post-transfection, cells were treated with 30 mM MG132 for 6 hours and lysed in buffer A (6 M guanidine-HCl, 0.1 M Na2HPO4/NaH2PO4, and 10 mM imidazole [pH 8.0]). After sonication, the lysates were incubated with nickel-nitrilotriacetic acid (Ni-NTA) beads (QIAGEN) for 3 hours at room temperature. Subsequently, the His pull-down products were washed twice with buffer A, twice with buffer ANTI (1 volume buffer A and 3 volumes buffer TI), and one time with buffer TI (25 mM Tris-HCl and 20 mM imidazole [pH 6.8]). The pull-down proteins were resolved by 2×SDS-PAGE for immunoblotting.

### Protein Half-Life Assays.

Cells were transfected or treated under indicated conditions. For half-life studies, cycloheximide (20 μg/ml, Sigma) was added to the medium. At indicated time points thereafter, cells were harvested and protein abundances were measured by immunoblot analysis.

### Cell Synchronization and FACS Analyses

Cells were synchronized with nocodazole arrest and double thymidine treatment as described previously (Wan et al. Developmental cell 29, 377-391 (2014)). Cells synchronized with nocodazole or double thymidine-arrest and release were collected at the indicated time points and stained with propidium iodide (Roche) according to the manufacturer's instructions. Cells were fixed by 70% ethanol at −20° C. overnight and washed 3 times using cold PBS. The samples were digested with RNase for 30 minutes at 37° C. and stained with propidium iodide (Roche) according to the manufacturer's instructions. Stained cells were sorted with BD FACSCanto™ II Flow Cytometer. The results were analyzed by ModFit LT 4.1 and FSC express 5 softwares.

### Real-Time RT-PCR Analyses

Total RNAs were extracted using the QIAGEN RNeasy mini kit, and reverse transcription reactions were performed using the ABI Taqman Reverse Transcription Reagents (N808-0234). After mixing the generated cDNA templates with primers/probes and ABI Taqman Fast Universal PCR Master Mix (4352042), reactions were performed with the ABI-7500 Fast Real-time PCR system and SYBR green qPCR Mastermix (600828) from Agilent Technologies Stratagene.

Human GAPDH:
Forward, 5′-GGAGCGAGATCCCTCCAAAAT-3′,
Reverse, 5′-GGCTGTTGTCATACTTCTCATGG-3′;
Mouse GAPDH:
Forward, 5′-AGGTCGGTGTGAACGGATTTG-3′,
Reverse, 5′-GGGGTCGTTGATGGCAACA-3′;
Human PD-L1:
Forward, 5′-TGGCATTTGCTGAACGCATTT-3′,
Reverse, 5′-TGCAGCCAGGTCTAATTGTTTT-3′;
Mouse PD-L1:
Forward, 5′-GCTCCAAAGGACTTGTACGTG-3′,
Reverse, 5′-TGATCTGAAGGGCAGCATTTC-3′;

### Generation of Cyclin D-Deficient MEFs

Cyclin D1^{-/-}, D2^{-/-}, D3^{-/-} and D1^{F/F}D2^{-/-}D3^{F/F}MEFs were derived from E13.5 mouse embryos as described previously.

### Generation of Mouse Tumors

Cyclin D1^{-/-} mice were mated with MMTV-c-Myc or MMTV-Wnt1 mice (from the Jackson Laboratory) yielding cyclin D1^{-/-}/MMTV-c-Myc or cyclin D1^{-/-}/MMTV-Wnt1, as well as control cyclin D1^{+/+}/MMTV-c-Myc or D1^{+/+}/MMTV-Wnt1 mice. Mammary tumors were dissected from multiparous females and snap-frozen.

MMTV-ErbB2 female mice (from the Jackson Laboratory), bred into a mixed C57BL/6 and 129Sv background, were treated with palbociclib or vehicle only for 6 weeks after detection of palpable tumors. Palbociclib was administered daily by gastric gavage (150 mg/kg of body weight); every two weeks the daily dose was lowered to 100 mg/kg for 2-3 days. Control mice were treated with vehicle (10% 0.1N HCl, 10% Cremaphor EL, 20% PEG300, 60% 50 mM citrate buffer pH 4.5) 10 ml/kg by gastric gavage. After 6 weeks, tumors were collected and snap-frozen in OCT.

Treatment of Wild-Type Mice with Palbociclib

6-weeks old C57BL/6 female mice (from the Jackson Laboratory) were treated with palbociclib (150 mg/kg body weight, by gastric gavage) or vehicle only for 7 days. Subsequently, organs were collected and analyzed by immunoblotting.

### Mouse Tumor Implantation

1×10⁵ B16-F10 or 2×10⁵ MC38 cells were injected subcutaneously into 6-weeks old C57BL/6 female mice (from the Jackson Laboratory). Starting one week later, mice were treated daily with palbociclib (150 mg/kg body weight, by gastric gavage) or vehicle only, for 7 days. Subsequently, tumors were collected and analyzed by FACS or immunoblotting. 1×10⁵ B16-F10 cells stably expressing SPOP WT or F102C mutant were injected subcutaneously into 6-weeks old C57BL/6 female mice (from the Jackson Laboratory). On day 3 after tumor cells were injected, control and PD-L1 mAb treatments were conducted by intra-peritoneal injection (200 μg/mouse in 200 μl HBSS saline buffer) every three days for a total of 3 injections. Subsequently, tumors were collected and analyzed by FACS.

### Immunofluorescence Staining of Tumor Tissues

TFM-embedded 10 μM-thick tumor tissue sections were fixed with 2% paraformaldehyde/PBS for 30 min, and permeabilized in 0.1% Triton X-100/PBS for 10 min. Tumor tissue sections were pre-blocked with 2% BSA/PBS for 45 min, then incubated with primary antibodies against PD-L1 (1:200), CD3 (Abcam, 1:250) for 2.5 hours at room temperature and followed with secondary anti-mouse antibodies conjugated with Alexa-fluor-568 (Invitrogen, 1:250) and anti-rabbit antibodies conjugated with Alexa-fluor-488 (Invitrogen, 1:250). Hoechst (life technology, 1:10,000) was used to stain nucleus. Tumor tissues were mounted with fluoromount-G® (SouthernBiotech) at 4° C. overnight. Tissue sections were examined with fluorescent microscope under a 20 x objective lens. CD3+ cell numbers were counted in an area of 5.95×10⁵ μm².

Single Cell Generation from Tumor Tissue and Flow Cytometry Analysis

Tumor tissues were minced and digested with 5 ml of 2 mg/ml collagenase (Sigma) in DMEM for 1 hour at 37° C. Cells were then collected by centrifuge and filtered through a 70 μm strainer in DMEM. Cell pellets were suspended and lysed in red blood cell lysis buffer for 5 min. The cells were then filtered through a 40 μm strainer in 1 x PBS with 2% BSA. 1 million cells were incubated with antibodies against PD-L1 (BD Biosciences, 1:100) conjugated with APC or antibodies against CD3 (Biolegend, 1:100) conjugated with APC or corresponding isotype IgG1 control at room temperature for 30 min. Cells were washed by 1×PBS with 2% BSA and analyzed by flow cytometry.

### In Vivo Experimental Therapy in MC38 and CT26 Mice Tumor Model

Animal studies were approved by Dana-Farber Cancer Institute Institutional Animal Care and Use Committee (IACUC; protocol number 04-047), and performed in accordance with guidelines established by NIH Guide for the care and use of laboratory animals. MC38 or CT26 tumors were established by subcutaneously injecting 1×10⁵ MC38 or CT26 tumor cells in 100 μl HBSS into the right flank of 6-week old C57BL/6 or BALB/c female mice (Jackson Lab, ME). Tumor sizes were measured every three days by caliper after implantation and tumor volume was calculated by length×width²×0.5. On day 7 after tumor cells were injected, animals were pooled and randomly divided into four groups with comparable average tumor size. Moreover, the lab members who measured the mice were blinded to the treatment groups. Mice were grouped into control antibody treatment, PD-1 mAb treatment, CDK4/6 inhibitor treatment, and PD-1 mAb plus CDK4/6 inhibitor treatment. As illustrated in FIG. 14A, control and PD-1 mAb treatments were conducted by intraperitoneal injection (200 μg/mouse in 200 μl HBSS saline buffer) every three days for a total of 8 injections. The CDK4/6 inhibitor treatment was given by oral gavage once a day with a dosage of 100 mg/kg for three weeks with a break every week for one day. For survival studies, animals were monitored for tumor volumes every three days for 120 days after initial treatment, until tumor volume exceeded 2000 mm³, or until tumor had ulcer with diameter reached 1 cm. Statistical analysis was conducted using the GraphPad Prism software (GraphPad Software, Inc., San Diego, Calif.). Kaplan-Meier curves and corresponding Gehan-Breslow-Wilcoxo tests were used to evaluate the statistical differences between groups in survival studies. p<0.05 was considered to be significant.

### OTHER EMBODIMENTS

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

1. A therapeutic combination comprising a cyclin D kinase 4/6 (CDK4/6) inhibitor and an anti-PD-L1 antibody and/or an anti-PD-1 antibody.

2. The therapeutic combination of claim 1, wherein the CDK4/6 inhibitor is palbociclib, ribociclib, abemaciclib or trilaciclib.

3. The therapeutic combination of claim 1, wherein the anti-PD-1 antibody is nivolumab, pembrolizumab, or pidilizumab.

4. The therapeutic combination of claim 1, wherein the anti-PD-L1 antibody is MPDL3280A, MEDI4736, BMS-936559, or MSB0010718C.

5. The therapeutic combination of claim 1, wherein the combination comprises a CDK4/6 inhibitor and an anti-PD-L1 or an anti-PD-1 antibody.

6. The therapeutic combination of claim 1, wherein the combination is formulated in a single composition or is formulated and administered separately.

7. The therapeutic combination of claim 1, wherein the combination comprises a CDK4/6 inhibitor and an anti-PD1 antibody.

8. The therapeutic combination of claim 1, wherein the CDK4/6 inhibitor is palbociclib.

9. A method of reducing tumor growth, the method comprising contacting a tumor cell with a cyclin D kinase 4/6 (CDK4/6) inhibitor and an anti-PD-L1 and/or an anti-PD-1 antibody, thereby reducing tumor growth.

10. A method of treating cancer in a subject, the method comprising administering to the subject a cyclin D kinase 4/6 (CDK4/6) inhibitor and an anti-PD-L1 and/or an anti-PD-1 antibody, thereby treating cancer in the subject.

11. The method of claim 9, wherein the CDK4/6 inhibitor is palbociclib, ribociclib, abemaciclib, or trilaciclib.

12. The method of claim 9, wherein the anti-PD-1 antibody is nivolumab, pembrolizumab, or pidilizumab.

13. The method of claim 9, wherein the anti-PD-L1 antibody is MPDL3280A, MEDI4736, BMS-936559, or MSB0010718C.

14. The method of claim 9, wherein the combination comprises a CDK4/6 inhibitor and an anti-PD-L1 or an anti-PD-1 antibody.

15. The method of claim 9, wherein the combination is formulated in a single composition or is formulated and administered separately.

16. The method of claim 7, wherein the cancer is selected from the group consisting of colon cancer, breast cancer, melanoma, prostate cancer, lung cancer, and head and neck cancer.

17. The method of claim 7, wherein treatment reduces tumor growth relative to a reference.

18. The method of claim 8, wherein treatment increases survival of the subject.

19. A kit comprising the therapeutic combination of claim 1.


SEQUENCE LISTING (32 sequences)

SEQ ID NO: 1 - protein, 176 aa, Homo sapiens
MRIFAVFIFMTYWHLLNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 2 - DNA, 3349 bp, Homo sapiens
ggcgcaacgctgagcagctggcgcgtcccgcgcggccccagttctgcgcagcttcccgaggctccgcaccagccgcgcttctgtccgcctgcagggcattccagaaagatgaggatatttgctgtctttatattcatgacctactggcatttgctgaacgccccatacaacaaaatcaaccaaagaattttggttgtggatccagtcacctctgaacatgaactgacatgtcaggctgagggctaccccaaggccgaagtcatctggacaagcagtgaccatcaagtcctgagtggtaagaccaccaccaccaattccaagagagaggagaagcttttcaatgtgaccagcacactgagaatcaacacaacaactaatgagattttctactgcacttttaggagattagatcctgaggaaaaccatacagctgaattggtcatcccagaactacctctggcacatcctccaaatgaaaggactcacttggtaattctgggagccatcttattatgccttggtgtagcactgacattcatcttccgtttaagaaaagggagaatgatggatgtgaaaaaatgtggcatccaagatacaaactcaaagaagcaaagtgatacacatttggaggagacgtaatccagcattggaacttctgatcttcaagcagggattctcaacctgtggtttaggggttcatcggggctgagcgtgacaagaggaaggaatgggcccgtgggatgcaggcaatgtgggacttaaaaggcccaagcactgaaaatggaacctggcgaaagcagaggaggagaatgaagaaagatggagtcaaacagggagcctggagggagaccttgatactttcaaatgcctgaggggctcatcgacgcctgtgacagggagaaaggatacttctgaacaaggagcctccaagcaaatcatccattgctcatcctaggaagacgggttgagaatccctaatttgagggtcagttcctgcagaagtgccctttgcctccactcaatgcctcaatttgttttctgcatgactgagagtctcagtgttggaacgggacagtatttatgtatgagtttttcctatttattttgagtctgtgaggtcttcttgtcatgtgagtgtggttgtgaatgatttcttttgaagatatattgtagtagatgttacaattttgtcgccaaactaaacttgctgcttaatgatttgctcacatctagtaaaacatggagtatttgtaaggtgcttggtctcctctataactacaagtatacattggaagcataaagatcaaaccgttggttgcataggatgtcacctttatttaacccattaatactctggttgacctaatcttattctcagacctcaagtgtctgtgcagtatctgttccatttaaatatcagctttacaattatgtggtagcctacacacataatctcatttcatcgctgtaaccaccctgttgtgataaccactattattttacccatcgtacagctgaggaagcaaacagattaagtaacttgcccaaaccagtaaatagcagacctcagactgccacccactgtccttttataatacaatttacagctatattttactttaagcaattcttttattcaaaaaccatttattaagtgcccttgcaatatcaatcgctgtgccaggcattgaatctacagatgtgagcaagacaaagtacctgtcctcaaggagctcatagtataatgaggagattaacaagaaaatgtattattacaatttagtccagtgtcatagcataaggatgatgcgaggggaaaacccgagcagtgttgccaagaggaggaaataggccaatgtggtctgggacggttggatatacttaaacatcttaataatcagagtaattttcatttacaaagagaggtcggtacttaaaataaccctgaaaaataacactggaattccttttctagcattatatttattcctgatttgcctttgccatataatctaatgcttgtttatatagtgtctggtattgtttaacagttctgtcttttctatttaaatgccactaaattttaaattcatacctttccatgattcaaaattcaaaagatcccatgggagatggttggaaaatctccacttcatcctccaagccattcaagtttcctttccagaagcaactgctactgcctttcattcatatgttcttctaaagatagtctacatttggaaatgtatgttaaaagcacgtatttttaaaatttttttcctaaatagtaacacattgtatgtctgctgtgtactttgctatttttatttattttagtgtttcttatatagcagatggaatgaatttgaagttcccagggctgaggatccatgccttctttgtttctaagttatctttcccatagcttttcattatctttcatatgatccagtatatgttaaatatgtcctacatatacatttagacaaccaccatttgttaagtatttgctctaggacagagtttggatttgtttatgtttgctcaaaaggagacccatgggctctccagggtgcactgagtcaatctagtcctaaaaagcaatcttattattaactctgtatgacagaatcatgtctggaacttttgttttctgctttctgtcaagtataaacttcactttgatgctgtacttgcaaaatcacattttctttctggaaattccggcagtgtaccttgactgctagctaccctgtgccagaaaagcctcattcgttgtgcttgaacccttgaatgccaccagctgtcatcactacacagccctcctaagaggcttcctggaggtttcgagattcagatgccctgggagatcccagagtttcctttccctcttggccatattctggtgtcaatgacaaggagtaccttggctttgccacatgtcaaggctgaagaaacagtgtctccaacagagctccttgtgttatctgtttgtacatgtgcatttgtacagtaattggtgtgacagtgttctttgtgtgaattacaggcaagaattgtggctgagcaaggcacatagtctactcagtctattcctaagtcctaactcctccttgtggtgttggatttgtaaggcactttatcccttttgtctcatgtttcatcgtaaatggcataggcagagatgatacctaattctgcatttgattgtcactttttgtacctgcattaatttaataaaatattcttatttattttgttacttggtacaccagcatgtccattttcttgtttattttgtgtttaataaaatgttcagtttaacatcccagtggagaaagttaaaaaa

SEQ ID NO: 3 - protein, 288 aa, Homo sapiens
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 4 - DNA, 9026 bp, Homo sapiens
agtttcccttccgctcacctccgcctgagcagtggagaaggcggcactctggtggggctgctccaggcatgcagatcccacaggcgccctggccagtcgtctgggcggtgctacaactgggctggcggccaggatggttcttaggtaggtggggtcggcggtcaggtgtcccagagccaggggtctggagggaccttccaccctcagtccctggcaggtcggggggtgctgaggcgggcctggccctggcagcccaggggtcccggagcgaggggtctggagggacctttcactctcagtccctggcaggtcggggggtgctgtggcaggcccagccttggcccccagctctgccccttaccctgagctgtgtggctttgggcagctcgaactcctgggttcctctctgggccccaactcctcccctggcccaagtcccctctttgctcctgggcaggcaggacctctgtcccctctcagccggtccttggggctgcgtgtttctgtagaatgacgggtcaggctggccagaaccccaaaccttggccgtggggagtctgcgtggcggctctgccttgcccaggcatccttggtcctcactcgagttttcctaaggatgggatgagccccatgtgggactaaccttggctttacgacgtcaaagtttagatgagctggtgatatttttctcattatatccaaagtgtacctgttcgagtgaggacagttcttctgtctccaggatccctcctgggtggggattgtgcccgcctgggtctctgcccagattccagggctctccccgagccctgttcagaccatccgtgggggaggccttggcctcactctcccggatcgaggagagagggagcctcttcctgggctgcccgtgaccctgggccctctgtgtacactgtgaccacagcccgctcctggaccctctgtgcccggctggccctctgtgcccagccagcctgcacctggggatgccaaggcctggggagggtggtttcacccaggccaagcctaagacagtccctctgggccctgctgggtaccggggtgtgacaccactgggaggacaagatgaggggcacccctggggccgccctgacaccccctcgaggctcctgccccgggggtcctggtgccccttcactgtggcaggcgactgggggttccccacctcggcccctctcccggggcctgctccccggcacctgaggcagcatccttgtcagggccgtgccttcctgcctcagcgccacctcttaaggttggcccgtgggtcactcaggactcagaactggagattctgggcaaaaggcaaagagcaaagggccaaaaggcatcccagggagacgactgcgggggaaccagagggcagaggggcgctcgtcacaggggagggggagctgagcgaggcaggaggggagccgagcctctccccccgtgtcccggctcttcaggcacgccctcgggacgccaccctccccgacccaggcgggaaagataagagcaaggtgtccgcagcctgacactcgtgcctcaggtgcccgcgcttgtgccggacaagactctcacaggtggcatgcctcggtttccccactggtaacagcacagggcactcagcaaggcgcagtgggcatgactggggtcctgtgggtcctgacccagatgtggccaccccggccgcagtggtcttcattccaggatgcctcttttccctcctgatctattcactgcgttcgccattcggtcattcccggggccaccactcccacctcaggtgtgtgcttcccttgtgttttatgagatatccccaacccggctgcttattggccccgtccgagggcaggagcataaataagagcctctgctttggcgtgggaccactgtgagctccagtcagcgctgccactgctgagctctgggccttcgacaggacttggccccttactgacttctccgtgtgctttgggtcatgggtgaggacgcctcctggcaaggctgcgtcctgaggattaaatcgggtcatctgtgaaaactacccagcccagcccctgacacttttttgtttgtttcttttagtgacagggtcttgctctgtcacccaggctggagtgcagtggtgtgatctcggctcactcgacctccggggctcaagcaattctcccacctctgcctccagagtagctgggactataggcacgtgccaccctgccaggctaatttcttccattttttttagagacagggtctcgctatgttccccaggctgggctcaaatggtcctcccacctcagcctccccaagtactgggattacaggcataagccactgcatctggcctccatgacacatatttttaaagtctgatttttaaagtcaaacttttgaagtcagattttaaacggactattttgaaaaatatacaaaaacgtttaaaaacaatgaatatccctcacctagaatcaataactaagaatattgacacatttgctttggggactgggcggctggagctgccatgacaaagctccgccgaccgagtggcttttaaacagagcctgccctctcgccgactgagggctggacgtgcaggatggagctccgcagggtcggctcccctgtgctctgaggggctctgctcagcctctcccggctgtggcttaaaaacagagcctgtcctcccgccgtggggggctggacatgcaggaccgaggggccacagggtcggctccctgtgctccgagagggctctgctcagcttctcctggctggggggttttgtggccaccctctgtgttcctgggttcagaagcatcccccaggctctgccttcatctctgcacgggtgactctgtacaggaagccaggcctgctggtcaatggccacccagccctgtgccctcatcttacctagtcccagctgccgtcaccctattcctaataaggccgccttctgaggtcatggggttaggacttccacataggaatctgtggggacacggttcggcccacagcccttcccacctccacacacacacacgactgtgaggagttggaagacctcactcctcacccctgccaggtcctctagggacaagctcgctgtcctcatcccagcacagcccgtgggacggtttccttgtccctaatgggaccacggtcagagatgccgggtctggtctgggccagcaggttcctccgcccggggcaggcagccttcttctgtgcgcttctggaaagcaatgtcctgtaatgcggtctctctgcgggagcacccccaccgccacctcacaggcctgttccacagccccgggatgggctctgtctccctcctgaccctgcatagggcacagccctctctcatcaacccacgatcctacgtggatccgagagggagcacctggggaaacaatggaatcccatagaaacaccccaaatctaacttgatccaggaccagccagtggtcacttctgaatattcaccttcctagtagacactaccagccaagggaggccaggaagccttcctggaggaggtggcctgaggactggggtgaggcaggccctgcgtgggggtcgccacccagcacccccacactgggtgggagccagtctctgagactggctgggggaggtgggagagggggctgcttgaactgcagacaccgaggtctagcccccaccccacccagccagttggtggaggcaggggaggccgaggggcccagctggacctgctccccggggtggattccaaaataggggggttggggggggcggaacaggagcccagggtcctggcttgaggcccagtggctgagggctggtgcaagccagacaggaaaagggttgagcctgtcagcgccagcacagatcaagtcaggagcaggtccctccaccaatgtgtgcaaataaatagcagctaagtttccagttacaagaacaatgcacagatggtcccagggacattgcggtgtggacacacagcggccattgtcctgtcgccagcacctcgccctacagctggggggtcccttagcacttcctagccatgcagggtccctgctcacagtacccgtgatgacttctgttcctcacctgcctgtctgtcccgacagctgcatggcagccctggcctgggagatggagaccccgaggggctgcctgcggtggtggggcccctgggtccccactgcattcccagaaacccagagggcagggcatttcccctgctctgtgccgagtccacccagccccagcctaggcccagtaagggctgcagcccaccctgtcccaggctgcctcccaggagccctcttggccctgatgccagaagcccatcttcctccattcaggcaggtctctgagtgccctggcctggctgcctgctggccctgagagtcacactaccccacagccctccttggtcaaaatccactctggagtggctggaagattccccgggcccacgccgcacacgcctatgcagggagcttcccctggccggccggcagacaagggcggtctcagagagggggctcacctcagcagccccttgtgtagctggccctcgcccctgccacctctgggaacaccaccaggaagctgggggacaggcacgcaggtgaaggaggcgagcgcttgtcagccgggaggccatgggcacagagggaacagggacaccctgggtggcctcaaggtcacttcaaacccctcactcgtcccctgggagggtgcccagtgaggttggcactaggagttggtcctggtcacatgacagacccacccacctctggtgtccagccagcacgccgtgggccagcctggctgcagggacacgagggcagcagccccctcctcctctgagctggttgctccttgagtcatcaccaccgcctgccacggaggccgcctgtcccaggaagcagagggaccgcagctgtggcaaccagggcctggtctctgtgtcacctcgctggggggccgtgcccaggcctgagacggaactgagtgacagtgcactgggtctgacagtgtggggctggcgccatgtttggggaaccctgtggcatgggacctgtgggtgagccgggaaaatcaccccgttgcatggcatctcgggcctggatcttaagcgcctgtgttggtgcctccgcctggcggaagagccgcgacccccacgttgccatgcgggtatcccaagccctgaccctggcaggcatatgtttcaggaggtccttgtcttgggagcccagggtcgggggccccgtgtctgtccacatccgagtcaatggcccatctcgtctctgaagcatctttgctgtgagctctagtccccactgtcttgctggaaaatgtggaggccccactgcccactgcccagggcagcaatgcccataccacgtggtcccagctccgagcttgtcctgaaaagggggcaaagactggaccctgagcctgccaaggggccacactcctcccagggctggggtctccatgggcagccccccacccacccagaccagttacactcccctgtgccagagcagtgcagacaggaccaggccaggatgcccaagggtcaggggctggggatgggtagcccccaaacagccctttctgggggaactggcctcaacggggaagggggtgaaggctcttagtaggaaatcagggagacccaagtcagagccaggtgctgtgcagaagctgcagcctcacgtagaaggaagaggctctgcagtggaggccagtgcccatccccgggtggcagaggccccagcagagacttctcaatgacattccagctggggtggcccttccagagcccttgctgcccgagggatgtgagcaggtggccggggaggctttgtggggccacccagccccttcctcacctctctccatctctcagactccccagacaggccctggaacccccccaccttctccccagccctgctcgtggtgaccgaaggggacaacgccaccttcacctgcagcttctccaacacatcggagagcttcgtgctaaactggtaccgcatgagccccagcaaccagacggacaagctggccgccttccccgaggaccgcagccagcccggccaggactgccgcttccgtgtcacacaactgcccaacgggcgtgacttccacatgagcgtggtcagggcccggcgcaatgacagcggcacctacctctgtggggccatctccctggcccccaaggcgcagatcaaagagagcctgcgggcagagctcagggtgacaggtgcggcctcggaggccccggggcaggggtgagctgagccggtcctggggtgggtgtcccctcctgcacaggatcaggagctccagggtcgtagggcagggaccccccagctccagtccagggctctgtcctgcacctggggaatggtgaccggcatctctgtcctctagctctggaagcaccccagcccctctagtctgccctcacccctgaccctgaccctccaccctgaccccgtcctaacccctgacctttgtgcccttccagagagaagggcagaagtgcccacagcccaccccagcccctcacccaggccagccggccagttccaaaccctggtggttggtgtcgtgggcggcctgctgggcagcctggtgctgctagtctgggtcctggccgtcatctgctcccgggccgcacgaggtaacgtcatcccagcccctcggcctgccctgccctaaccctgctggcggccctcactcccgcctccccttcctccacccttccctcaccccaccccacctccccccatctccccgccaggctaagtccctgatgaaggcccctggactaagaccccccacctaggagcacggctcagggtcggcctggtgaccccaagtgtgtttctctgcagggacaataggagccaggcgcaccggccagcccctggtgagtctcactcttttcctgcatgatccactgtgccttccttcctgggtgggcagaggtggaaggacaggctgggaccacacggcctgcaggactcacattctattatagccaggaccccacctccccagcccccaggcagcaacctcaatccctaaagccatgatctggggccccagcccacctgcggtctccgggggtgcccggcccatgtgtgtgcctgcctgcggtctccaggggtgcctggcccacgcgtgtgcccgcctgcggtctctgggggtgcccggcccacatatgtgcctgcctgcggtctccaggtgtgcccggcccatgcgtgtgcccacctgcgagggcgtggggtgggcttggtcatttcttatcttacattggagacaggagagcttgaaaagtcacattttggaatcctaaatctgcaagaatgccagggacatttcagagggggacattgagccagagaggaggggtggtgtccccagatcacacagagggcagtggtgggacagctcagggtaagcagctcatagtggggggcccaggttcggtgccggtactgcagccaggctgtggagccgcgggcctccttcctgcggtgggccgtggggctgactccctctccctttctcctcaaagaaggaggacccctcagccgtgcctgtgttctctgtggactatggggagctggatttccagtggcgagagaagaccccggagccccccgtgccctgtgtccctgagcagacggagtatgccaccattgtctttcctagcggaatgggcacctcatcccccgcccgcaggggctcagctgacggccctcggagtgcccagccactgaggcctgaggatggacactgctcttggcccctctgaccggcttccttggccaccagtgttctgcagaccctccaccatgagcccgggtcagcgcatttcctcaggagaagcaggcagggtgcaggccattgcaggccgtccaggggctgagctgcctgggggcgaccggggctccagcctgcacctgcaccaggcacagccccaccacaggactcatgtctcaatgcccacagtgagcccaggcagcaggtgtcaccgtcccctacagggagggccagatgcagtcactgcttcaggtcctgccagcacagagctgcctgcgtccagctccctgaatctctgctgctgctgctgctgctgctgctgctgcctgcggcccggggctgaaggcgccgtggccctgcctgacgccccggagcctcctgcctgaacttgggggctggttggagatggccttggagcagccaaggtgcccctggcagtggcatcccgaaacgccctggacgcagggcccaagactgggcacaggagtgggaggtacatggggctggggactccccaggagttatctgctccctgcaggcctagagaagtttcagggaaggtcagaagagctcctggctgtggtgggcagggcaggaaacccctccacctttacacatgcccaggcagcacctcaggccctttgtggggcagggaagctgaggcagtaagcgggcaggcagagctggaggcctttcaggcccagccagcactctggcctcctgccgccgcattccaccccagcccctcacaccactcgggagagggacatcctacggtcccaaggtcaggagggcagggctggggttgactcaggcccctcccagctgtggccacctgggtgttgggagggcagaagtgcaggcacctagggccccccatgtgcccaccctgggagctctccttggaacccattcctgaaattatttaaaggggttggccgggctcccaccagggcctgggtgggaaggtacaggcgttcccccggggcctagtacccccgccgtggcctatccactcctcacatccacacactgcacccccactcctggggcagggccaccagcatccaggcggccagcaggcacctgagtggctgggacaagggatcccccttccctgtggttctattatattataattataattaaatatgagagcatgctaagga

SEQ ID NO: 5 - DNA, 21 bp, Artificial Sequence
  Description of Artificial Sequence Synthetic primer
ggagcgagatccctccaaaat

SEQ ID NO: 6 - DNA, 23 bp, Artificial Sequence
  Description of Artificial Sequence Synthetic primer
ggctgttgtcatacttctcatgg

SEQ ID NO: 7 - DNA, 21 bp, Artificial Sequence
  Description of Artificial Sequence Synthetic primer
aggtcggtgtgaacggatttg

SEQ ID NO: 8 - DNA, 19 bp, Artificial Sequence
  Description of Artificial Sequence Synthetic primer
ggggtcgttgatggcaaca

SEQ ID NO: 9 - DNA, 21 bp, Artificial Sequence
  Description of Artificial Sequence Synthetic primer
tggcatttgctgaacgcattt

SEQ ID NO: 10 - DNA, 22 bp, Artificial Sequence
  Description of Artificial Sequence Synthetic primer
tgcagccaggtctaattgtttt

SEQ ID NO: 11 - DNA, 21 bp, Artificial Sequence
  Description of Artificial Sequence Synthetic primer
gctccaaaggacttgtacgtg

SEQ ID NO: 12 - DNA, 21 bp, Artificial Sequence
  Description of Artificial Sequence Synthetic primer
tgatctgaagggcagcatttc

SEQ ID NO: 13 - protein, 31 aa, Homo sapiens
RLRKGRMMDVKKCGIQDTNSKKQSDTHLEET

SEQ ID NO: 14 - protein, 30 aa, Mus musculus
RKQVRMLDVEKCGVEDTSSKNRNDTQFEET

SEQ ID NO: 15 - protein, 15 aa, Homo sapiens
FIRRDFLLDEANGLL

SEQ ID NO: 16 - protein, 15 aa, Mus musculus
FIRRDFLLDEANGLL

SEQ ID NO: 17 - protein, 15 aa, Rattus norvegicus
FIRRDFLLDEANGLL

SEQ ID NO: 18 - protein, 15 aa, Bos taurus
FIRRDFLLDEANGLL

SEQ ID NO: 19 - protein, 15 aa, Danio rerio
FIRRDFLLDEANGLL

SEQ ID NO: 20 - protein, 15 aa, Xenopus laevis
FIRRDFLLDEANGLL

SEQ ID NO: 21 - protein, 13 aa, Homo sapiens
MSRVPSPPPPAEM

SEQ ID NO: 22 - protein, 13 aa, Homo sapiens
ILAARSPVFSAMF

SEQ ID NO: 23 - protein, 13 aa, Mus musculus
MSRVPSPPPPAEM

SEQ ID NO: 24 - protein, 13 aa, Mus musculus
ILAARSPVFSAMF

SEQ ID NO: 25 - protein, 13 aa, Rattus norvegicus
MSRVPSPPPPAEM

SEQ ID NO: 26 - protein, 13 aa, Rattus norvegicus
ILAARSPVFSAMF

SEQ ID NO: 27 - protein, 13 aa, Bos taurus
MSRVPSPPPPAEM

SEQ ID NO: 28 - protein, 13 aa, Bos taurus
ILAARSPVFSAMF

SEQ ID NO: 29 - protein, 13 aa, Danio rerio
MSRVPSPPPPAEM

SEQ ID NO: 30 - protein, 13 aa, Danio rerio
ILAARSPVFSAMF

SEQ ID NO: 31 - protein, 13 aa, Xenopus laevis
MSRVPSPPPPAEM

SEQ ID NO: 32 - protein, 13 aa, Xenopus laevis
ILAARSPVFSAMF